SEQUENCE LISTING (29 sequences)

SEQ ID NO: 1 - DNA, 28 bp, synthetic construct
  source 1..28: mol_type = other DNA
catcttcttcaaggacgacggcaactac

SEQ ID NO: 2 - DNA, 28 bp, synthetic construct
  source 1..28: mol_type = other DNA
gttcttctgcttgtcggcggtgatatag

SEQ ID NO: 3 - DNA, 45 bp, synthetic construct
  source 1..45: mol_type = other DNA
tcgagggcgacaccctggtgaaccgcatcggctgaagggcatcga

SEQ ID NO: 4 - skipped

SEQ ID NO: 5 - RNA, 42 nt, synthetic construct
  source 1..42: mol_type = other RNA
taatttctactaagtgtagatgagggcgatgccacctacggc

SEQ ID NO: 6 - RNA, 42 nt, synthetic construct
  source 1..42: mol_type = other RNA
taatttctactaagtgtagattagccttcgggcatggcggac

SEQ ID NO: 7 - RNA, 42 nt, synthetic construct
  source 1..42: mol_type = other RNA
taatttctactaagtgtagatcgtgcagctcgccgaccacta

SEQ ID NO: 8 - RNA, 42 nt, synthetic construct
  source 1..42: mol_type = other RNA
taatttctactaagtgtagatatcgcgcttctcgttggggtc

SEQ ID NO: 9 - RNA, 42 nt, synthetic construct
  source 1..42: mol_type = other RNA
taatttctactaagtgtagatgtagttaatgattaacccgcc

SEQ ID NO: 10 - RNA, 42 nt, synthetic construct
  source 1..42: mol_type = other RNA
taatttctactaagtgtagatctcagggcggactgggtgctc

SEQ ID NO: 11 - RNA, 42 nt, synthetic construct
  source 1..42: mol_type = other RNA
taatttctactaagtgtagatcgtcgccgtccagctcgacca

SEQ ID NO: 12 - RNA, 42 nt, synthetic construct
  source 1..42: mol_type = other RNA
taatttctactaagtgtagatcaacagccacaaggtctatat

SEQ ID NO: 13 - RNA, 42 nt, synthetic construct
  source 1..42: mol_type = other RNA
taatttctactaagtgtagatgatgttgtggcgggtcttgaa

SEQ ID NO: 14 - RNA, 42 nt, synthetic construct
  source 1..42: mol_type = other RNA
taatttctactaagtgtagatgtcgcccggcctcagtgagcg

SEQ ID NO: 15 - skipped

SEQ ID NO: 16 - DNA, 747 bp, synthetic construct
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 17 - protein, 248 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 18 - DNA, 1911 bp, synthetic construct
  source 1..1911: mol_type = other DNA
atgtctagattagataaaagtaaagtgattaacagcgcattagagctgcttaatgaggtcggaatcgaaggtttaacaacccgtaaactcgcccagaagcttggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaacgcgttatatgcactcagcgctgtggggcattttaccttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtggggactcaggaggcggtggatccggtggaggcggttctggcggtggaggctctggaggtggcggatcgggcggaggtggctctagatctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtat

SEQ ID NO: 19 - protein, 481 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGDSGGGGSGGGGSGGGGSGGGGSGGGGSRSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 20 - RNA, 21 nt, synthetic construct
  source 1..21: mol_type = other RNA
ccgtgttccagcacgacgact

SEQ ID NO: 21 - RNA, 21 nt, synthetic construct
  source 1..21: mol_type = other RNA
tcccggtcgaagctctcgggc

SEQ ID NO: 22 - RNA, 21 nt, synthetic construct
  source 1..21: mol_type = other RNA
tgcagctgctcgccggcggtc

SEQ ID NO: 23 - RNA, 21 nt, synthetic construct
  source 1..21: mol_type = other RNA
ccgacgcacatatcgaggtgg

SEQ ID NO: 24 - RNA, 21 nt, synthetic construct
  source 1..21: mol_type = other RNA
gggtcgtgctcatgtaccgct

SEQ ID NO: 25 - RNA, 21 nt, synthetic construct
  source 1..21: mol_type = other RNA
gcccgacgacgatgccggcga

SEQ ID NO: 26 - RNA, 21 nt, synthetic construct
  source 1..21: mol_type = other RNA
ggacgcccgcaagatccgcga

SEQ ID NO: 27 - RNA, 21 nt, synthetic construct
  source 1..21: mol_type = other RNA
cggcgccccgccgctggcgat

SEQ ID NO: 28 - RNA, 21 nt, synthetic construct
  source 1..21: mol_type = other RNA
ggcgtagccctaccgcaccgc

SEQ ID NO: 29 - RNA, 21 nt, synthetic construct
  source 1..21: mol_type = other RNA
cgcggcgagctgtgcgtccgt

(19) United States
(12) Patent Application Publication
(10) Pub. No.: US 2025/0327802 A1
(43) Pub. Date: Oct. 23, 2025

(54) Methods of Evaluating Virus-Producing Cells

(71) Applicant: Bruker Cellular Analysis, Inc., Emeryville, CA (US)

(72) Inventors: Jason C. Briggs, Pleasanton, CA (US); Leqian Liu, El Cerrito, CA (US); Nathan J. Ver Heul, Oakland, CA (US); Chen Sun, Emeryville, CA (US); Eric Chun-Jen Shiue, Westford, MA (US); Chih-Yun Hsia, Holliston, MA (US); Alexander J. Mastroianni, Alameda, CA (US); Eric K. Sackmann, Berkeley, CA (US); Jason M. McEwen, El Cerrito, CA (US); Peyton Shieh, Emeryville, CA (US); Qiong Gao, Emeryville, CA (US); J. Tanner Nevill, El Cerrito, CA (US); Volker L.S. Kurz, Oakland, CA (US); Michelle R. Murphy, Walnut Creek, CA (US); Kellen C. Mobilia, Livermore, CA (US); Brittany R. Benlian, Richmond, CA (US); Lauren Ashley Washburn, San Francisco, CA (US)

(73) Assignee: Bruker Cellular Analysis, Inc., Emeryville, CA (US)

(21) Appl. No.: 19/204,872

(22) Filed: May 12, 2025

Related U.S. Application Data
(63) Continuation of application No. PCT/US23/80178, filed on Nov. 16, 2023.
(60) Provisional application No. 63/426,307, filed on Nov. 17, 2022.
(60) Provisional application No. 63/538,742, filed on Sep. 15, 2023.

(51) Int. Cl. G01N 33/569; B01L 3/00; C12N 15/86

(52) U.S. Cl. CPC G01N 33/56983; B01L 3/502761; C12N 15/86; B01L 2200/0647; B01L 2300/042; B01L 2300/12; B01L 2400/0409; B01L 2400/0424; B01L 2400/0457; C12N 2750/14143; C12N 2750/14152; G01N 2333/015

(57) ABSTRACT

Method of evaluating a virus-producing cell on a microfluidic device is described therein. The method comprises culturing the virus-producing cell thereby producing a viral particle in a chamber of the microfluidic device; and evaluating a productivity of the virus-producing cell in producing the viral particle. Additionally, method for preserving a subset of biological micro-objects within a microfluidic device is also described herein so that the subset of biological micro-objects can be protected from being affected by the assays performed on the microfluidic device.

### CROSS-REFERENCE TO RELATED APPLICATIONS

This application is a continuation of International Application No. PCT/US2023/080178, filed Nov. 16, 2023, which claims the benefit of priority of U.S. Provisional Application Nos. 63/538,742, filed Sep. 15, 2023, and 63/426,307, filed Nov. 17, 2022, each of which is incorporated by reference herein in its entirety for any purpose.

### SEQUENCE LISTING

The present application contains a Sequence Listing which has been submitted electronically in XML format. Said XML copy, created on Nov. 16, 2023, is named “01149-0026-00PCT-ST26.xml” and is 28,770 bytes in size. The information in the electronic format of the sequence listing is incorporated herein by reference in its entirety.

### BACKGROUND OF THE INVENTION

Viral vectors are useful scientific tools in both academic and industrial sectors for delivering a target gene into cells. Various types of viruses, including adeno-associated viruses (AAV), adenoviruses, retroviruses, lentiviruses, etc., are widely used in basic research and areas such as vaccine developments and gene therapy, because of their flexibility, safety, stability, non-pathogenicity, tissue selectivity, and low immunogenicity. The production of viral vectors is at the heart of the success of viral vector-dependent products. An effective production can reduce the overall manufacture cost and lower the dose required for treatment. Therefore, there is constantly a need for a method of screening a virus-producing cell having desired productivity.

### SUMMARY OF THE INVENTION

In a first aspect, a method for preserving a subset of biological micro-objects within a microfluidic device is provided. The method comprises providing a microfluidic device, wherein the microfluidic device comprises a microfluidic circuit material defining a flow region and a plurality of chambers fluidically connecting to the flow region, and further wherein the plurality of chambers comprises a first chamber and a second chamber, and further wherein a plurality of biological micro-objects is disposed within the first chamber; moving a first subset of the plurality of biological micro-objects into the second chamber; designating one of the first chamber and the second chamber as a preserving chamber and the other as an assay chamber; and forming a first in situ-generated cap within the preserving chamber, wherein the first in situ-generated cap comprises a porosity to selectively block passage between the preserving chamber and the flow region.

In a second aspect, a method of evaluating a virus-producing cell on a microfluidic device is provided. The microfluidic device comprising a virus-producing cell, wherein the microfluidic device further comprises a microfluidic circuit material defining a flow region and a chamber fluidically connecting to the flow region, and wherein the virus-producing cell is disposed in the chamber. The method comprises: culturing the virus-producing cell under conditions suitable for producing a viral particle in the chamber; and evaluating a productivity of the virus-producing cell in producing the viral particle.

### BRIEF DESCRIPTION OF THE DRAWINGS

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 illustrates an exemplary workflow implementing the methods of the present disclosure.

FIGS. 7A-7J illustrates a process of splitting clone within a microfluidic device. FIG. 7A shows disposing a cell into one of the two chambers of a culture unit. FIG. 7B shows the disposed cell expands into a clonal population within the chamber. FIG. 7C shows treating the cells to let them detach from the surface of the chamber. FIG. 7D illustrates moving a cell of the clonal population into another chamber of the culture unit. FIG. 7E shows culturing the cells in both chamber and forming an in situ-generated cap 730 to seal one of the chambers. FIG. 7F shows introducing a helper virus to induce the production of viral particles. FIG. 7G shows the cells in chamber 711 become round shape indicating that the infection by helper virus and the production of viral particles affect the viability of the cells. FIG. 7H illustrates assays are performed in chamber 711 but not chamber 712 because chamber 712 is capped. FIG. 7I shows that a second in situ-generated cap 750 is formed to cap chamber 711. FIG. 7J shows the in situ-generated cap 730 of chamber 712 is removed and cells therewithin are exported.

FIGS. 7K-7R shows images corresponding to FIGS. 7A-7J. Please note that chamber 711 is now the chamber on the right and chamber 712 is the chamber on the left. FIG. 7K corresponds to FIG. 7A showing a cell is disposed in chamber 711. FIG. 7L corresponds to FIG. 7B showing the disposed cell is expended into a clonal population. FIG. 7M corresponds to FIG. 7C showing the cells are de-associated from the surface of chamber 711 before splitting. FIG. 7N corresponds to FIG. 7E showing a subset of the clonal population is moved to chamber 712 while the in situ-generated cap 730 is not formed yet. FIG. 7O corresponds to FIG. 7E showing a subset of the clonal population is moved to chamber 712 and the in situ-generated cap 730 is formed.

FIG. 7P corresponds FIG. 71 showing a second in situ-generated cap 750 is formed. FIG. 7Q corresponds to FIG. 7J showing the in situ-generated cap 730 is removed and before cells are exported. FIG. 7R corresponds to FIG. 7J showing the in situ-generated cap 730 is removed and cells are exported.

FIGS. 8A-8D are images showing a process of splitting clone within a microfluidic device using DEP force. FIG. 8A shows a pattern of the DEP electrode regions was illuminated within the chamber to activate DEP force. FIG. 8B shows cells are moved out of the first chamber by the activated DEP force. FIG. 8C shows cells are moved via the flow region to the second chamber by the activated DEP force. FIG. 8D shows that some cells are moved to the second chamber while some cells are retained in the original, first chamber.

FIG. 9 illustrates various types of in situ-generated barriers defining transit areas according to the present disclosure.

FIG. 10A shows an image of in situ-generated barriers corresponding to the in situ-generated barrier 924 shown in FIG. 9.

FIG. 10B shows an image of in situ-generated barriers corresponding to the in situ-generated barrier 922 shown in FIG. 9.

FIG. 10C shows an image of in situ-generated barriers corresponding to the in situ-generated barrier 923 shown in FIG. 9.

FIGS. 11A-11B shows splitting clone using gravity. FIG. 11A shows tilting the microfluidic device to let the cells within a chamber drop into the flow region. FIG. 11B shows tilting the microfluidic device to let the cells in the flow region drop into the chamber.

FIG. 12 illustrates various types of in situ-generated separating elements facilitating splitting clone according to the present disclosure.

FIG. 13 illustrates various types of in situ-generated cap according to the present disclosure.

FIGS. 14A-14C shows detecting a payload of a viral vector using isothermal amplification (Recombinase polymerase amplification, RPA). FIG. 14A shows images taken before the Recombinase polymerase amplification (RPA) assay is performed, including a brightfield image showing sequestration pens and cells therewithin (top) and a fluorescent image showing the fluorescent signal associated with a probe used in the RPA. FIG. 14B shows a fluorescent image taken after the 1st loading and the 2nd loading were completed but before the 3rd loading was performed-before the isothermal amplification is initiated. FIG. 14C shows a fluorescent image taken 10 minutes after the isothermal amplification is initiated. The arrows indicate a chamber comprising GFP+ cells (producer cells)

FIG. 15 shows the distribution of intensity of the fluorescent signals detected from the cells in the chambers tested in the RPA assay. Each dot represents a signal obtained from a chamber. The dotted line shows a cut-off value of the top 62 clonal populations. The diamond signs show the four producer clonal populations, which are all among the top 62 clonal populations.

FIG. 16 shows a plot of DETECTR signal (the difference between the pre-assay signal and the post-assay signal) and GFP signal obtained from a DETECTR assay. Each dot represents a signal obtained from a chamber. The pentagon dots represent the chambers selected for qPCR confirmation, and the arrows indicate the chambers that the cells therein are verified as GFP+ cells via qPCR.

FIG. 17A and FIG. 17B shows an image of the beginning of the DECTECTR assay and an image after 10 minutes of the DECTECTR assay respectively.

FIGS. 18A-18C shows graphical representations of the results of a DETECTR assay using NEM treatment and C rich probe. FIG. 18A shows the pre-assay signals. FIG. 18B shows the post-assay signals. FIG. 18C shows the DETECTR signals (the difference between the pre-assay signal and the post-assay signal). Each dot represents a signal obtained from a chamber.

FIG. 19 shows graphical representations of a physical titer assay accoridng to the present disclosure, including assay signals distributions of transfected cells (Top) and untransfected cells (Bottom).

FIG. 20 shows graphical representations of a genetic titer assay accoridng to the present disclosure, including assay signals distributions of transfected cells (Top) and untransfected cells (Bottom).

FIG. 21A shows graphical representations of results of the rtPCR conducted to examine the induction efficiency of doxycycline, a AAV vector encoding tTA, and a plasmid encoding tTA (“Transfection”). Each induction was repeated once. The rtPCR was performed to target the genome of the viral particle produced. Therefore, the lower the Ct value, the higher the production of the viral particle of interest.

FIG. 21B shows graphical representations of results of the rtPCR conducted to examine the induction efficiency of doxycycline, and a AdV vector encoding tTA (“Ad_bypass)”. Each induction was repeated twice. The rtPCR was performed to target the genome of the viral particle produced. Therefore, the lower the Ct value, the higher the production of the viral particle of interest.

FIG. 21C shows graphical representations of results of the rtPCR conducted to examine the induction efficiency of doxycycline, a plasmid encoding tTA, and a plasmid encoding sctTA. Each induction was repeated twice. The rtPCR was performed to target the genome of the viral particle produced. Therefore, the lower the Ct value, the higher the production of the viral particle of interest.

FIG. 22 shows graphical representations of the fraction of mCherry positive chambers of four microfluidic devices. Each device was induced with a different inducing agent or was not induced. The results demonstrate the transduction efficiency of an AdV viral vector at high MOI and low MOI, respectively, in capped and uncapped chambers. The chip induced with doxycycline and the chip that was not induced by any inducing agent (the “negative control”) are presented for comparison.

FIG. 23 shows graphical representations of the fraction of capsid positive chambers of four microfluidic devices using the physical titer assay of the present disclosure. Each of the four microfluidic devices was induced with a different inducing agent or was not induced. The results demonstrate the induction efficiency of an AdV viral vector at high MOI and low MOI, respectively, in capped and uncapped chambers. The chip induced with doxycycline and the chip that was not induced by any inducing agent (the “negative control”) are presented for comparison.

FIG. 24A shows population histograms for each cell line in capped or uncapped sequestration pens. The histograms plot percentage of pens (chambers) with DETECTR assay scores. The higher percentage of pens at higher scores indicates higher production rendered by the AdV viral vector encoding tTA.

FIG. 24B shows population histograms for each cell line in capped or uncapped sequestration pens. The histograms plot percentage of pens (chambers) with DETECTR assay scores. The higher percentage of pens at higher scores indicates higher production rendered by doxycycline.

FIG. 25A shows graphical representations of the fraction of capsid positive beads of uncapped chambers of four microfluidic devices using the physical titer assay of the present disclosure at Day 3 after induction. Each of the four microfluidic devices was induced with AAV high MOI, AAV low MOI, doxycycline, or was not induced.

FIG. 25B shows graphical representations of the fraction of capsid positive beads of capped chambers of four microfluidic devices using the physical titer assay of the present disclosure at Day 3 after induction. Each of the four microfluidic devices was introduced with AAV high MOI, AAV low MOI, doxycycline, or was not induced.

FIG. 25C shows graphical representations of the fraction of capsid positive beads of capped chambers of four microfluidic devices using the physical titer assay of the present disclosure at Day 5 after induction. Each of the four microfluidic devices was induced with AAV high MOI, AAV low MOI, doxycycline, or was not induced.

FIG. 26 shows graphical representations of the fraction of mCherry positive chambers of two microfluidic devices culturing producer cell line 3D or a parental cell line, respectively. Each microfluidic device was induced with a different inducing agent, including doxycycline or plasmid encoding tTA.

FIG. 27 shows graphical representations of the fraction of capsid positive chambers of two microfluidic devices culturing producer cell line 3D or a parental cell line, respectively. Each microfluidic device was induced with a different inducing agent including doxycycline or plasmid encoding tTA.

FIG. 28 illustrates schematics of using biotin to compete with desthiobiotin on binding with streptavidin. The concept shown in these schematics can be applied to attach a molecule of interest on a surface and release it at a later time point.

### DETAILED DESCRIPTION OF THE INVENTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25% or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10⁻⁵⁰, 10⁻⁷⁵, 10-100, 20-100, 20-150, 20-200, 50-200, 50-250, or 50-300 microliters. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 microliters, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or nonselectively. In one nonlimiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Either type of binding may be understood to be selective. Capture beads containing moieties/molecules may bind nonselectively when binding of structurally different but physico-chemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion, and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g., perfusing).

The phrase “substantially no flow” refers to a rate of flow of a fluidic medium that, when averaged over time, is less than the rate of diffusion of components of a material (e.g., an analyte of interest) into or within the fluidic medium. The ratio of a rate of flow of a component in a fluidic medium (i.e., advection) divided by the rate of diffusion of such component can be expressed by a dimensionless Peclet number. Thus, a region within a microfluidic device that experiences substantially no flow in one in which the Peclet number is less than 1. The Peclet number associated with a particular region within the microfluidic device can vary with the component or components of the fluidic medium being considered (e.g., the analyte of interest), as the rate of diffusion of a component or components in a fluidic medium can depend on, for example, temperature, the size, mass, and/or shape of the component(s), and the strength of interactions between the component(s) and the fluidic medium. In certain embodiments, the Peclet number associated with a particular region of the microfluidic device and a component located therein can be 0.95 or less, 0.9 or less, 0.85 or less, 0.8 or less, 0.75 or less, 0.7 or less, 0.65 or less, 0.6 or less, 0.55 or less, 0.5 or less, 0.4 or less, 0.3 or less, 0.2 or less, 0.1 or less, 0.05 or less, 0.01 or less, 0.005 or less, or 0.001 or less.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, “pen” or “penning” refers to disposing micro-objects within a chamber (e.g., a sequestration pen) within the microfluidic device. Forces used to pen a micro-object may be any suitable force as described herein such as dielectrophoresis (DEP), e.g., an optically actuated dielectrophoretic force (OEP); gravity; magnetic forces; or tilting. In some embodiments, penning a plurality of micro-objects may reposition substantially all the micro-objects. In some other embodiments, a selected number of the plurality of micro-objects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and introduced into a chamber by penning.

As used herein, “unpen” or “unpenning” refers to repositioning micro-objects from within a chamber, e.g., a sequestration pen, to a new location within a flow region, e.g., a microfluidic channel, of the microfluidic device. Forces used to unpen a micro-object may be any suitable force as described herein such as dielectrophoresis, e.g., an optically actuated dielectrophoretic force; gravity; magnetic forces; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “export” or “exporting” refers to repositioning micro-objects from a location within a flow region, e.g., a microfluidic channel, of a microfluidic device to a location outside of the microfluidic device, such as a 96 well plate or other receiving vessel. The orientation of the chamber(s) having an opening to the microfluidic channel permits easy export of micro-objects that have been positioned or repositioned (e.g., unpenned from a chamber) to be disposed within the microfluidic channel. Micro-objects within the microfluidic channel may be exported without requiring disassembly (e.g., removal of the cover of the device) or insertion of a tool into the chamber(s) or microfluidic channel to remove micro-objects for further processing.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

A “viral vector” is a nucleic acid that can be incorporated into a viral particle. A viral vector includes at least those sequences required in cis for replication and packaging. For instance, inverted terminal repeats (ITRs) are essential elements for replicating a viral vector of adeno-associated viruses in host cells. The ITRs need not be the wild-type nucleotide sequences, and may be altered, e.g., by the insertion, deletion or substitution of nucleotides, so long as the sequences provide for functional rescue, replication and packaging. Rep and cap proteins can be encoded by the vector or supplied in trans as occurs when rep and/or cap gene is replaced by a heterologous sequence. The viral vector can also include a target gene or payload (i.e., the genetic material to be delivered).

A “viral particle,” interchangeable with a virion, capsid, or head, is a viral capsid protein coat/shell encapsulating or without encapsulating a viral vector therewithin. Usually, a viral particle is able to infect a host cell and transfer a target gene to the host cell if the targe gene is encapsulated. The viral particle of the present disclosure can be derived from an adeno-associated virus (AAV), adenovirus (AdV), retrovirus, or lentivirus.

A “full” viral particle, virion, capsid or head means a complete viral particle comprising a viral vector encapsulated within a viral capsid protein coat/shell. The viral vector can be a native genome including rep, cap and ITRs or a recombinant DNA, in which rep and/or cap segments are replaced by a heterologous segment. Usually, a full viral particle should be able to infect a host cell and transfer the DNA to the host cell. The “full” viral particle, virion, capsid or head is interchangeable with “particles encapsulating DNA,” “particles encapsulating DNA,” or similar phrases.

An “empty” viral particle, virion, capsid or head means a viral capsid protein shell in the form of a viral particle but lacking a viral vector encapsulated within a viral capsid protein coat/shell. The viral vector can be a native genome (such as rep, cap, and ITRs) or a recombinant DNA as discussed above. “Empty” does not exclude the possibility that the viral particle encapsulates substances other than a viral vector. The “empty” viral particle, virion, capsid or head is interchangeable with “particles lacking encapsulated DNA,” “particles lacking encapsulated DNA,” or similar phrases.

The term “packaging efficiency” used herein refers to the efficiency of a producer cell in producing viral particles encapsulating the viral vector or target gene of interest (i.e., a “full” viral particle). Packaging efficiency can be presented as a percentage of full particles versus all viral particles produced by the producing viral particles. Alternatively, packaging efficiency can also be presented inversely as a percentage of empty particles versus all viral particles produced by the producing viral particles.

“Viral helper functions” refer to virus-derived coding sequences which can be expressed to provide viral gene products that, in turn, function in trans for productive viral replicaiton and packaging. Thus, viral helper functions include the major open reading frames, rep, and cap. The Rep expression products have been shown to possess many functions, including, among others: recognition, binding and nicking of the virus origin of DNA replication; DNA helicase activity; and modulation of transcription from the viral (or other heterologous) promoters. The Cap expression products supply necessary packaging functions. Viral helper functions can be used to complement viral functions in trans that are missing from viral vectors.

A recombinant virus means a virus that has been genetically altered, e.g., by the addition or insertion of a heterologous sequence into a viral vector.

A “host cell” means any type of cell usable as recipient of a viral vector. The term includes the progeny of the original cell which has been transfected. The progeny of a single parental cell may not necessarily be completely identical in morphology or in genomic or total DNA complement as the original parent, due to natural, accidental, or deliberate mutation.

A “producer cell” or a “virus-producing cell” used herein refers to a host cell that is prepared to produce the viral particles of interest. A producer cell is a host cell transfected with a viral genome of the viral particle to be produce. The viral genome comprises a payload flanked with two ITRs. The producer cell also carries a helper gene of the viral particle to be produced, such as the replication gene (e.g., rep gene) or capsid gene (cap gene), which is essential for the replication of the viral particle. The produced viral particles can be secreted by or retained in the producer cells. The term “producer cell” includes the progeny of the original cell which has been transfected. The progeny of a single parental cell may not necessarily be completely identical in morphology or in genomic or total DNA complement as the original parent, due to natural, accidental, or deliberate mutation.

### Viral Vectors

The efficiency of viral vector production is critical to the success of research and viral vector-dependent products. Typically, viral vectors are produced by a producer cell transfected with viral genes essential for replication. A producer cell with excellent productivities of viral particles is invaluable, especially for scaled-up production needed for industrial applications. However, there is no effective way in the field to provide a high throughput screening of producer cells.

On top of that, the packaging of replicated viral vector into the produced viral particles is not always effective. That said, the packaging might fail resulting in “empty” viral particles (i.e., viral particles do not carry the viral vector), which do not have much value to the intended applications. The higher the empty percentage of the collected viral particles, the lower the value they provide. The packaging efficiency is particularly important for medicinal products using viral vector because a product comprising high percentage of empty viral particles would require higher dose to reach the intended efficacy. Therefore, there is also a need to evaluate the packaging efficiency of a candidate producer cell.

### Evaluation of Virus-Producing Cells

The present disclosure provides methods of evaluating a virus-producing cell on a microfluidic device. The microfluidic device can be as described herein. The method comprises culturing the virus-producing cell thereby producing a viral particle in the chamber; and evaluating a productivity of the virus-producing cell.

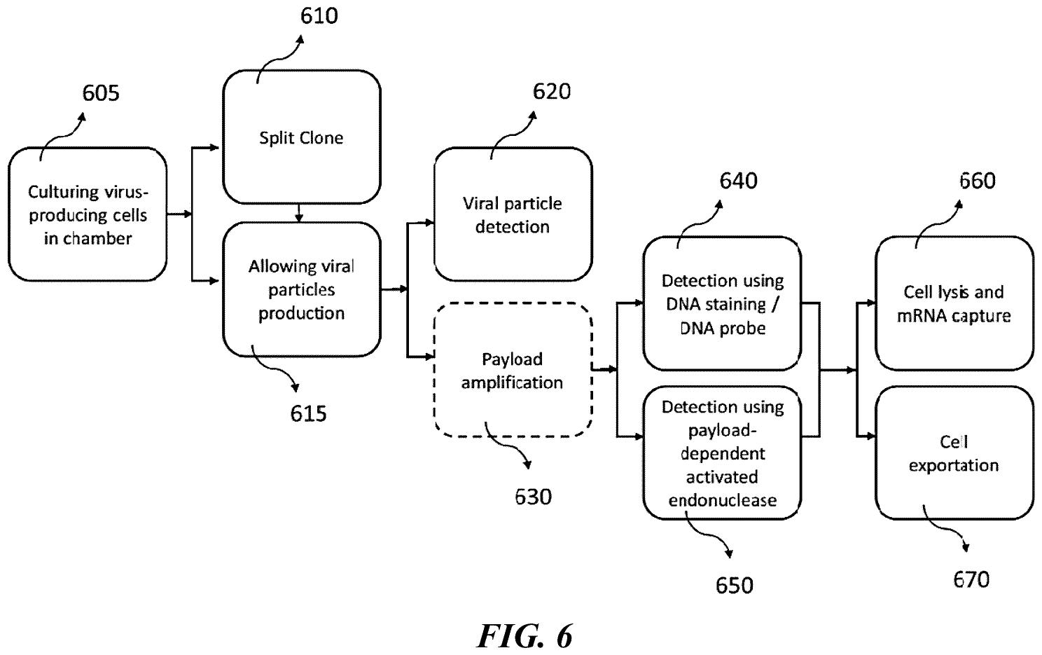

FIG. 6 shows an exemplary workflow of the present disclosure. The workflow comprises culturing the virus-producing cell in the chamber (e.g., sequestration pen) of the microfluidic device (Box 605). In some embodiments, culturing the virus-producing cell comprises introducing a fluidic medium comprising the virus-producing cell into the flow region of the microfluidic device and disposing the virus-producing cell into a chamber. The microfluidic device can comprise a plurality of chambers, and culturing the virus-producing cell can comprise introducing a fluidic medium comprising a plurality of virus-producing cells into the flow region of the microfluidic device and disposing a single cell of the plurality of virus-producing cells into a respective chamber of the plurality of chambers. In some embodiments, culturing the virus-producing cell comprises perfusing a culture medium into the flow region of the microfluidic device. Preferably, the perfusing is continued to allow the virus-producing cell to grow and expand into a clonal population. Then, the method comprises allowing the virus-producing cell to produce a viral particle (Box 615). In certain embodiments, allowing the virus-producing cell to produce a viral particle comprises inducing the virus-producing cell to produce the viral particle.

In some embodiments, the virus-producing cell expands into a clonal population therefrom in the chamber, and before allowing viral particle production (Box 615), a first subset of the clonal population is moved and preserved in an empty chamber of the microfluidic device while a second subset of the clonal population is retained in the original chamber (Box 610). The chambers contain cells of a single clonal population or derived from a single clonal population are designated as a culture unit. At least one chamber of the culture unit is then used for inducing the viral particle production and for performing an assay for evaluating a productivity of the virus-producing cells (i.e. an assay chamber), while at least one chamber of the culture unit is used for preserving cells from being affected by the assay performed in the assay chamber (i.e., a preserving chamber).

In some embodiments, the assay chamber is undergone either a physical titer assay (detecting the amount of the viral particles produced) or a genetic titer assay (detecting the payload of the viral vector) or both. In some embodiments, a physical titer assay is performed in the assay chamber and then a genetic titer assay is performed thereafter. In some embodiments, the virus-producing cell is lysed to release the viral particles produced and accumulated in the cytoplasm of the cell.

The physical titer assay comprises detecting the viral particles produced by the virus-producing cell (Box 620). In some embodiments, the genetic titer assay can comprise amplifying the payload (Box 630). Amplifying the payload may increase the efficiency of detecting the payload. In some embodiments, the payload can be detected using a nucleic acid dye (e.g., a DNA dye) or a probe configured to hybridize with the payload (Box 640). In some embodiments, the amplification of the payload is an isothermal amplification coupled with detecting the payload using a probe.

Alternative, in some embodiment, detecting the payload can be performed by using a payload-dependent activated endonuclease and a detectable probe (Box 650). As used herein, a “payload-dependent activated endonuclease” refers to an endonuclease that is activated upon associating or binding a payload. In some embodiments, detecting the payload using a payload-dependent activated endonuclease is using a CRISPR-based approach where the payload-dependent activated endonuclease is a Cas protein comprising a nucleotide acid having a sequence complementary to at least a portion of the payload.

After identifying the virus-producing cell with desired productivity of viral particle (i.e., a cell of interest), the cell can be exported for subsequent analysis and application (Box 670). Alternative, in some embodiments, the virus-producing cell is engineered with genetic barcode. Therefore, the cell can be lysed, and the mRNA thereof can be collected for detecting the barcode and thereby verifying the identity of the cell (Box 660). The lysis treatment of the cell can be performed on chip (e.g., in the chamber) or off chip after the cell of interest is exported.

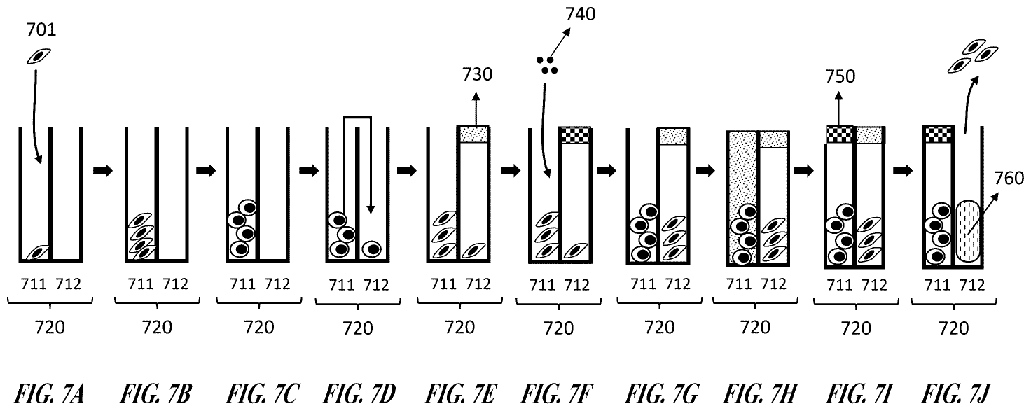

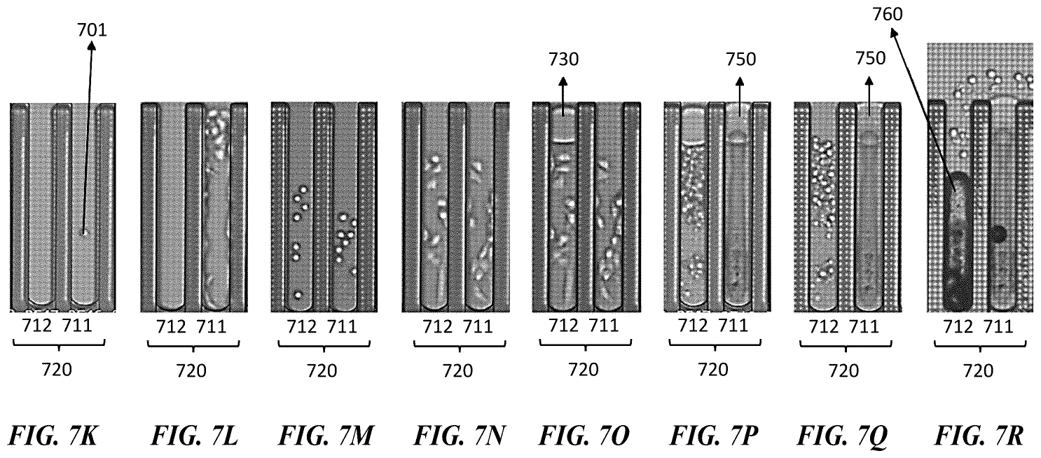

FIGS. 7A-7J are schematics showing a culture unit comprising two chambers of a microfluidic device for describing an exemplary workflow of the present disclosure with detailed process of splitting clone (Box 610 of FIG. 6). FIGS. 7K-7R shows corresponding images. The process of splitting clone is briefly described here and described in more detailed later in this specification.

FIG. 7A and FIG. 7B illustrates Box 605 of FIG. 6. A virus-producing cell 701 is introduced into a chamber 711 (FIG. 7A and corresponding FIG. 7K) where the virus-producing cell is cultured and expanded into a clonal population (FIG. 7B and corresponding FIG. 7L). FIG. 7C and FIG. 7D illustrate Box 610 of FIG. 6. Cells are dissociated from the surface before being split (FIG. 7C), and then a subset of the clonal population is moved to the chamber 712 (FIG. 7D and corresponding FIG. 7M). Cells are cultured and adhere on the surfaces of the chamber 711 and chamber 712 respectively (FIG. 7N). The cells illustrated in this example are adherent cells, while in some other embodiments, the cells can also be non-adherent (i.e., suspension) cells.

An in situ-generated cap 730 is then formed in chamber 712 (FIG. 7E and corresponding FIG. 7O) thereby separating or isolating the cells in chamber 712 from the flow region and chamber 711. After that, in the example shown in FIG. 7F, an inducing agent 740 is introduced to the microfluidic device to induce the production of viral particles. As illustrated, the inducing agent 740 is able to enter chamber 711 but is blocked from enter chamber 712 capped by the in situ-generated cap 730. While being induced to produce viral particles, cells in chamber 711 are dying (becoming round shape and suspending in the chamber 711) (FIG. 7G). In the meantime, because chamber 712 is capped by the in situ-generated cap 730, the cells in the chamber 712 are not affected by the induction happening in the chamber 711. Assay reagents can be introduced to assay the cells and the produced viral particles within chamber 711 (FIG. 7H). The assays can comprise the assays depicted in Box 620, Box 630, Box 640, and/or Box 650 of FIG. 6.

After a desired virus-producing cell is identified by performing the assay, the identified cell can be lysed as depicted in Box 660 of FIG. 6 or exported for further analysis or uses as depicted in Box 670 of FIG. 6. In the example shown in FIG. 7I, before exporting the identified cell, another in situ-generated cap 750 can be formed to cap chamber 711 (FIG. 71 and corresponding FIG. 7P). The in situ-generated cap 750 can prevent from accidentally exporting cells remaining in chamber 711. Then, the in situ-generated cap 730 is dissolved (FIG. 7J and correspond FIG. 7Q-7R) for exporting cells in chamber 712. In the example shown in FIG. 7J, FIG. 7Q, and FIG. 7R, a laser beam (not shown in the figures) is projected at the distal end of chamber 712 to generate a bubble 760, which dislodges the cells in chamber 712, thereby exporting those cells.

Producer cell and induction of viral particle production. For safety concern, the viral particles (viral vector) used for gene therapy are designed to be non-replicating. Usually, the viral particles are designed to carry a payload flanked with two ITRs but does not carry the helper genes essential for replication such as capsid gene (e.g., cap gene) and replication gene (rep gene). In some embodiments, the helper genes are carried by the producer cell constructed for producing the viral particle of interest, and the expression of the helper genes is controlled by an inducible promoter. Therefore, in such embodiment, inducing the virus-producing cell to produce the viral particle comprises inducing the inducible promoter.

In some embodiments, inducing the inducible promoter comprises contacting the virus-producing cell with an inducing agent. The inducing agent can be introduced together with a fluidic medium into a flow region of a microfluidic device, and is allowed to diffuse into the chamber or is disposed into the chamber where the virus-producing cell is disposed.

Small molecule inducer. The inducing agent can be a small molecule inducer. The small molecule inducer can be introduced into the flow region and then is allowed to diffuse into the chamber where the virus-producing cell is disposed. When a culture unit comprising a preserving chamber (e.g., see chamber 712 of FIG. 7E) and an assay chamber is used, the small molecule inducer is preferably blocked by a hydrogel cap disposed at the opening of the preserving chamber so that the virus-producing cell preserved in the preserving chamber will not be affected by the inducing agent. Therefore, in order to protect cells within the preserving chamber from being affected by the inducing agent, the hydrogel cap capping the preserving chamber should have a permeability lower than the size of the small molecule inducer. Alternatively, the small molecule inducer can be conjugated with a carrier thereby having a larger size and will not be able to pass through the hydrogel cap. The conjugate is however allowed to enter the assay chamber and can enter or be taken up by the virus-producing cell and release the small molecule inducer within the cells.

In some embodiments, the small molecule inducer does not bind to the inducible promoter directly but is configured to form a complex with a transactivator. The formed complex is capable of binding to the inducible promoter thereby initiating the expression of the helper gene resulting in viral particle production. The transactivator can be expressed by the virus-producing cell. In some embodiments, the virus-producing cell is engineered to constitutively express the transactivator.

In certain embodiments, the inducible promoter comprises a tetracycline response element (TRE). Such tetracycline response element allows controlling the expression of the helper gene by Tetracycline (Tet) inducible expression system. In such embodiments, the small molecule inducer can be a tetracycline, such as a doxycycline, and the transactivator is a reverse tetracycline-controlled transactivator (rtTA). The rtTA itself is not able to bind to the TRE but is configured to bind to tetracycline (e.g., doxycycline) resulting in a Doxy-rtTA complex, which is capable of binding to the TRE thereby initiating gene expression. In other words, the gene expression is only turned on in the presence of tetracycline, hence a Tet-on system. In some embodiments, the virus-producing cell is engineered to consecutively express the rtTA.

Carrier for expressing an inducer. In some embodiments, the inducing agent comprises a carrier carrying a nucleic acid encoding an inducer. The inducer is configured to bind to the inducible promoter thereby inducing the inducible promoter. In certain embodiments, contacting the virus-producing cell with an inducing agent comprises allowing the nucleic acid encoding the inducer to enter the virus-producing cell. In certain embodiments, inducing the inducible promoter further comprises expressing the inducer within the virus-producing cell. The carrier can be a plasmid, a viral particle, or a bead.

In some embodiments, the inducer is a product of a viral helper gene, which can be from an adenovirus or a herpesvirus. Some commonly used viral helper gene of adenovirus comprises E1A, E1B55K, E2A, E4orf6, VA RNA, or a combination thereof, and some commonly used viral helper gene of herpesvirus comprises UL5, UL8, UL52, ICP8, or a combination thereof. In certain embodiments, a helper viral particle carrying the viral helper gene is introduced to the flow region and is allowed to diffuse into the chamber to contact the virus-producing cell. The mechanism of using AdV to induce AAV particle production and release from a producer cell is described in Maurer, A. C., & Weitzman, M. D. (2020). Adeno-Associated Virus Genome Interactions Important for Vector Production and Transduction. Human gene therapy, 31 (9-10), 499-511. https://doi.org/10.1089/hum 2020.069, which is incorporated herein by reference.

Alternatively, a triple-plasmid transfection approach can be perform where a virus-producing cell can be transfected with a rep/cap plasmid (e.g., a plasmid carrying the AAV cap gene and rep gene), a recombinant viral genome plasmid (e.g, a plasmid carrying a payload flanked with ITRs), and an AdV helper plasmid. The replication of the viral vector can be mediated by the AdV helper plasmid without the need of helper virus infection. In some embodiments, the replicated viral particles can exit the virus-producing cell without a lysis treatment. While in some other embodiments, the produced viral particles might accumulate within the cytoplasm of the virus-producing cell, and a lysis treatment to lyse the virus-producing cell might be needed to facilitate collecting and assaying the viral particles. The triple-plasmid transfection approach is described in Nguyen, T et al., (2021). Mechanistic model for production of recombinant adeno-associated virus via triple transfection of HEK293 cells. Molecular therapy. Methods & clinical development, 21, 642-655. https:/doi ora/10 1016/.omun. 2021.04.006, which is incorporated herein by reference.

In some embodiments, the inducible promoter comprises a tetracycline response element (TRE). Such tetracycline response element allows controlling the expression of the helper gene by Tetracycline (Tet) inducible expression system. In such embodiments, a carrier carrying a nucleotide acid encoding a tetracycline-controlled transactivator (tTA) or a single-chain tetracycline-controlled transactivator (sctTA) is used as an inducing agent. The tTA and the sctTA are used as inducers of the present disclosure.

Tetracycline-controlled transactivator (tTA) is a recombinant protein created by fusing N-terminal transposon Tn10-derived TetR (tetracycline repressor) with the C-terminal domain of VP16 (virion protein 16), which is an important transactivator of Herpes simplex virus. Normally, tTA acts as a homodimer comprising two identical units, each comprises one tetR domain and one VP16 domain. Single-chain tetracycline-controlled transactivator (sctTA) comprises the VP16 domain and two TetR domines in one molecule, which is designed to allow the two TetR domains to undergo intermolecular dimerization. Both tTA and sctTA can bind to the TRE and initiate the gene expression in the absence of tetracycline. In other words, using a carrier carrying a nucleic acid encoding tTA or sctTA as the inducing agent can bypass the need of using tetracycline (e.g., doxycycline) for induction.

In the embodiments that the virus-producing cell is engineered to consecutively express rtTA as described above, using sctTA can be beneficial compared with using tTA as the inducer. Similar to tTA, rtTA also acts as a homodimer comprising two identical units, each comprises a rTetR and a VP16. The rTetR is a mutated form of TetR so that, in the absence of tetracycline, it loses its ability to bind to the TRE. Without wishing being bound by theories, when tTA is expressed within a virus-producing cell that also express rtTA, there is a concern that the expressed tTA will form heterodimer with the expressed rtTA, and the induction efficiency will be affected. The concern can be addressed by using sctTA as the inducer. Since the sctTA undergoes intermolecular dimerization, there is lower chance for forming heterodimers with the rtTA.

In certain embodiments, the nucleic acid can comprise SEQ ID NO: 16, which encodes tTA comprising an amino acid sequence of SEQ ID NO: 17; or the nucleic acid can comprise SEQ ID NO: 18, which encodes sctTA comprising an amino acid sequence of SEQ ID NO: 19.

| | |
|---|---|
| SEQ ID NO: 16 | SEQ ID NO: 17 |
| (DNA sequence of tTA) | (amino acid sequence of tTA) |
| atgtctagactggacaagagcaaagtcataaactctgct | MSRLDKSKVINSALELLNEVGIEGLTT |
| ctggaattactcaatgaagtcggtatcgaaggcctgac | RKLAQKLGVEQPTLYWHVKNKRALL |
| gacaaggaaactcgctcaaaagctgggagttgagcag | DALAIEMLDRHHTHFCPLEGESWQDF |
| cctaccctgtactggcacgtgaagaacaagcgggccc | LRNNAKSFRCALLSHRDGAKVHLGTR |
| tgctcgatgccctggcaatcgagatgctggacaggcat | PTEKQYETLENQLAFLCQQGFSLENAL |
| catacccacttctgccccctggaaggcgagtcatggca | YALSAVGHFTLGCVLEDQEHQVAKEE |
| agactttctgcggaacaacgccaagtcattccgctgtgc | RETPTTDSMPPLLRQAIELFDHQGAEP |
| tctcctctcacatcgcgacggggctaaagtgcatctcg | AFLFGLELIICGLEKQLKCESGGPADA |
| gcacccgcccaacagagaaacagtacgaaaccctgg | LDDFDLDMLPADALDDFDLDMLPAD |
| aaaatcagctcgcgttcctgtgtcagcaaggcttctccc | ALDDFDLDMLPG |
| tggagaacgcactgtacgctctgtccgccgtgggcca | |
| ctttacactgggctgcgtattggaggatcaggagcatca | |
| agtagcaaaagaggaaagagagacacctaccaccga | |
| ttctatgcccccacttctgagacaagcaattgagctgttc | |
| gaccatcagggagccgaacctgccttccttttcggcct | |
| ggaactaatcatatgtggcctggagaaacagctaaagt | |
| gcgaaagcggcgggccggccgacgcccttgacgatt | |
| ttgacttagacatgctcccagccgatgcccttgacgact | |
| ttgaccttgatatgctgcctgctgacgctcttgacgatttt | |
| gaccttgacatgctccccgggtaa | |
| SEQ ID NO: 18 | SEQ ID NO: 19 |
| (DNA sequence of sctTA) | (amino acid sequence of sctTA) |
| atgtctagattagataaaagtaaagtgattaacagcgca | MSRLDKSKVINSALELLNEVGIEGLTT |
| ttagagctgcttaatgaggtcggaatcgaaggtttaaca | RKLAQKLGVEQPTLYWHVKNKRALL |
| acccgtaaactcgcccagaagcttggtgtagagcagc | DALAIEMLDRHHTHFCPLEGESWQDF |
| ctacattgtattggcatgtaaaaaataagcgggctttgct | LRNNAKSFRCALLSHRDGAKVHLGTR |
| cgacgccttagccattgagatgttagataggcaccatac | PTEKQYETLENQLAFLCQQGFSLENAL |
| tcacttttgccctttagaaggggaaagctggcaagatttt | YALSAVGHFTLGCVLEDQEHQVAKEE |
| ttacgtaataacgctaaaagttttagatgtgctttactaag | RETPTTDSMPPLLRQAIELFDHQGAEP |
| tcatcgcgatggagcaaaagtacatttaggtacacggc | AFLFGLELIICGLEKQLKCESGDSGGG |
| ctacagaaaaacagtatgaaactctcgaaaatcaattag | GSGGGGSGGGGSGGGGSGGGGSRSRL |
| cctttttatgccaacaaggtttttcactagagaacgcgtta | DKSKVINSALELLNEVGIEGLTTRKLA |
| tatgcactcagcgctgtggggcattttaccttaggttgcg | QKLGVEQPTLYWHVKNKRALLDALA |
| tattggaagatcaagagcatcaagtcgctaaagaagaa | IEMLDRHHTHFCPLEGESWQDFLRNN |
| agggaaacacctactactgatagtatgccgccattatta | AKSFRCALLSHRDGAKVHLGTRPTEK |
| cgacaagctatcgaattatttgatcaccaaggtgcagag | QYETLENQLAFLCQQGFSLENALYAL |
| ccagccttcttattcggccttgaattgatcatatgcggatt | SAVGHFTLGCVLEDQEHQVAKEERET |
| agaaaaacaacttaaatgtgaaagtggggactcagga | PTTDSMPPLLRQAIELFDHQGAEPAFL |
| ggcggtggatccggtggaggcggttctggcggtgga | FGLELIICGLEKQLKCESGGPADALDD |
| ggctctggaggtggcggatcgggcggaggtggctcta | FDLDMLPADALDDFDLDMLPADALD |
| gatctagactggacaagagcaaagtcataaactctgct | DFDLDMLPG |
| ctggaattactcaatgaagtcggtatcgaaggcctgac | |
| gacaaggaaactcgctcaaaagctgggagttgagcag | |
| cctaccctgtactggcacgtgaagaacaagcgggccc | |
| tgctcgatgccctggcaatcgagatgctggacaggcat | |
| catacccacttctgccccctggaaggcgagtcatggca | |
| agactttctgcggaacaacgccaagtcattccgctgtgc | |
| tctcctctcacatcgcgacggggctaaagtgcatctcg | |
| gcacccgcccaacagagaaacagtacgaaaccctgg | |
| aaaatcagctcgcgttcctgtgtcagcaaggcttctccc | |
| tggagaacgcactgtacgctctgtccgccgtgggcca | |
| ctttacactgggctgcgtattggaggatcaggagcatca | |
| agtagcaaaagaggaaagagagacacctaccaccga | |
| ttctatgcccccacttctgagacaagcaattgagctgttc | |
| gaccatcagggagccgaacctgccttccttttcggcct | |
| ggaactaatcatatgtggcctggagaaacagctaaagt | |
| gcgaaagcggcgggccggccgacgcccttgacgatt | |
| ttgacttagacatgctcccagccgatgcccttgacgact | |
| ttgaccttgatatgctgcctgctgacgctcttgacgatttt | |
| gaccttgacatgctccccgggtaactaagtaaggatcc | |
| agacatgataagatacattgatgagtttggacaaaccac | |
| aactagaatgcagtgaaaaaaatgctttatttgtgaaattt | |
| gtgatgctattgctttatttgtaaccattataagctgcaata | |
| aacaagttaacaacaacaattgcattcattttatgtttcag | |
| gttcagggggaggtgtgggaggttttttaaagcaagta | |
| aaacctctacaaatgtggtatggctgattatgatcctgca | |
| agcctcgtcgtcctggccggaccacgctatctgtgcaa | |
| ggtccccggccccggacgcgcgctccatgagcagag | |
| cgcccgccgccgaggcgaagactcgggcggcgccc | |
| tgcccgtcccaccaggtcaacaggcggtaaccggcct | |
| cttcatcgggaatgcgcgcgaccttcagcatcgccggc | |
| atgtccccctggcggacgggaagtat | |

The expression of tTA or sctTA in the producer cell can be controlled by a promoter. The promoter can be those are configured for driving gene expression in mammalian cells. In some embodiments, the promoter is a constitutive promoter to provide consecutive expression of tTA. In certain embodiments, the promoter can be a cytomegalovirus (CMV) promoter, an EFIA promoter, a CBh promoter, a murine phosphoglycerate kinase (mPGK) promoter, or a Rous sarcoma virus (RSV) promoter. In some embodiments, the promoter is a controllable or inducible promoter. The controllable or inducible promoter can be controlled or inducible by inducible transcription factor-based regulation, Notch regulation, light regulation, or FKB12-FLP or Cre ER based regulation. In some embodiments, the promoter is not a tetracycline inducible promoter.

In some embodiments, the carrier is a plasmid or a bead. In such embodiments, contacting the virus-producing cell with the inducing agent comprises disposing the carrier into the chamber. Disposing the carrier into the chamber can be performed by using DEP force, gravity, centrifugation, or a combination thereof.

In certain embodiments, the carrier is a plasmid, and a fluidic medium comprising the plasmid is introduced to the flow region of the microfluidic device and is disposed into the chambers where the producer cells were disposed. Preferably, when a preserving chamber and an assay chamber are used as described herein the plasmid will be blocked by the hydrogel cap and will not be able to enter the preserving chamber. In some embodiments, disposing the plasmid comprises using centrifugation to effectively move the plasmid from the flow region into the chambers. In certain embodiments, more than one times of introducing and disposing the plasmid is performed. For example, a fluidic medium comprising the plasmid is prepared, and a portion of it is introduced into the flow region, and the plasmid contained in the portion is moved into the chamber by centrifugation. Then, another portion of the fluidic medium is introduced into the flow region, and the plasmid contained therein is moved into the chamber by centrifugation.

In other embodiments, a linear nucleic acid comprising the nucleic acid encoding the tTA is introduced into the producer cells. In such embodiments, a positive charge polymer is introduced to disrupt the integrity of the cell membrance thereby increasing the uptake of the linear nucleic acid into the producer cells. The positive charge polymer includes but not limited to DEAE-dextran, polyethylenmine, poly(β-amino ester), poly(lactic acid), polycarbonates, or polyurethanes, conjugated polymers, protein, or polysaccharides,. In some embodiments, the linear nucleic acid is attached to a bead, and introducing the linear nucleic acid is conducted by introducing the bead. In some embodiments, the induction procedure further comprises releasing the attached linear nucleic acid from the bead.

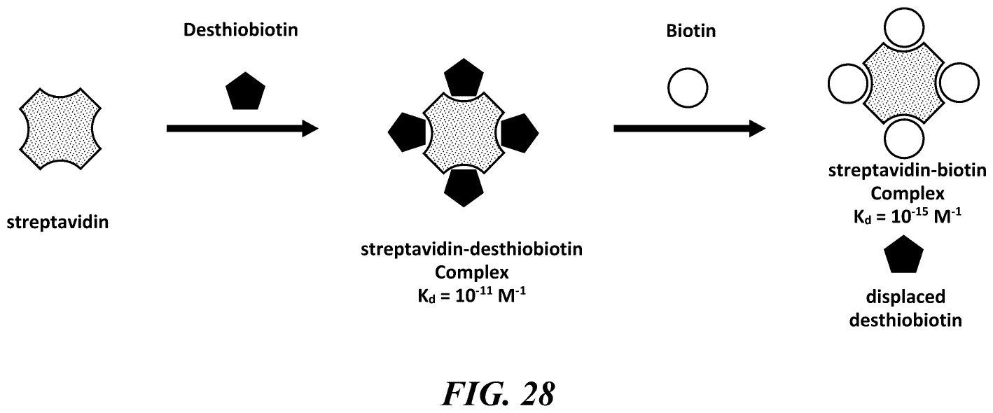

In certain embodiments, the bead is coated with a reactive moiety, which is configured to couple with a first anchoring moiety conjugated with linear nucleic acid so that the linear nucleic acid can be attached to the bead via the coupling between the reactive moiety and the first anchoring moiety. In such embodiments, releasing the linear nucleic acid can comprise introducing a releasing factor comprising a second anchoring moiety configured to couple with the reactive moiety as an affinity higher than the affinity between the first anchoring moiety and the reactive moiety. The second anchoring moiety therefore competes with the first anchoring moiety on binding with the reactive moiety thereby releasing the linear nucleic acid. For example, the reactive moiety on the bead can be a streptavidin. The first anchoring moiety can be a desthiobioin, and the second anchoring moiety can be a biotin. The binding between streptavidin and desthiobioin has a dissociation constant (K_{D}) of about 10⁻¹¹M⁻¹, and that of streptavidin and biotin is about 10⁻¹⁵M⁻¹. Accordingly, in this example, the linear nucleic acid is conjugated with desthiobioin in order to anchor on the bead. A releasing factor comprising biotin can complete the streptavidin on the bead with the desthiobioin thereby releasing the linear nucleic acid from the bead (FIG. 28). In some embodiments, evaluating the productivity of the virus-producing cell comprises determining the productivity and comparing the productivity with a threshold. The threshold can be obtained from a benchmark virus-producing cell line or from a virus-producing cell line previously identified to have desired productivity. In some embodiments, a plurality of virus-producing cells can be evaluated using the methods of the present disclosure, and the productivities thereof can be compared with each other to identify the virus-producing cells of better productivities.

In some embodiments, evaluating the productivity comprises evaluating the amounts of viral particles produced by the virus-producing cell in a given period. In some embodiments, evaluating the productivity comprises evaluating the packaging (i.e., the packaging efficiency) of viral vectors in the produced viral particles. In certain embodiments, evaluating the productivity comprises evaluating both the amount of the produced viral particles and the packaging thereof.

Evaluating the amounts of viral particle production. The amounts of viral particles produced can be evaluated by disposing a virus-capturing structure comprising a capture moiety into the chamber, wherein the virus-capturing capture moiety is configured to bind the viral particle; introducing a reporter molecule comprising a binding component and a detectable label into the flow region, wherein the binding component is configured to bind a viral particle of interest; and detecting a signal associated with the detectable label. In some embodiments, the virus-capturing structure is a capture bead or an in situ-generated capture structure.

As used herein, “capture moiety” and “binding component” respectively is a chemical or biological species, functionality, or motif that provides a recognition site for a viral particle of interest. In some embodiments, the capture moiety and/or the binding component comprises a peptide or a protein. In certain embodiments, the capture moiety. In certain embodiments, the binding component is an antibody, and the reporter molecule is an antibody having a detectable label.

In the embodiments that the virus-capturing structure is a capture bead, a surface of the capture bead is coated with the capture moiety. In some embodiments, the capture bead can be made of any suitable material, such as polymer, metal, ceramic, glass, or any combination thereof. In some embodiments, the capture bead can be magnetic or may not be magnetic. In certain embodiments, the capture bead is a bead coated with streptavidin covalently or noncovalently, and the capture moiety comprises a biotin functionality, which can couple with the streptavidin of the bead. In some embodiments, except for streptavidin/biotin, other coupling groups can be used, including but not limited to, biotin/avidin, biotin/NeutrAvidin, and digoxygenin/anti-digoxygenin.

In the embodiments that the virus-capturing structure is an in situ-generated capture structure, the in situ-generated capture structure comprises a solidified polymer network comprising a functionalized site, wherein the functionalized site is configured to associate with the capture moiety. In some embodiments, the functionalized site comprises a streptavidin functionality, and capture moiety comprises a biotin functionality, which can couple with the streptavidin functionality of the in situ-generated capture structure. In some embodiments, except for streptavidin/biotin, other coupling groups can be used, including but not limited to, biotin/avidin, biotin/NeutrAvidin, and digoxygenin/anti-digoxygenin. In some embodiments, the in situ-generated capture structure is as described in WO2017100347 filed on Dec. 7, 2016, which is incorporated herein by reference.

The amounts of viral particles produced can be evaluated by detecting a signal associated with the detectable label. A relatively amount of the viral particle production can be determined based on a saturation signal. In some embodiments, a saturation signal can be pre-determined based on the amount of capture moieties existing on the virus-capturing structure. In some embodiments, a saturation signal can be pre-determined by incubating the virus-capturing structure with a virus suspension of which the concentration of the viral particles is known.

Evaluating the packaging of the viral particle produced. The viral particle produced by a virus-producing cell might be an empty viral particle, that is, without carrying the viral vector or payload of interest. The present disclosure provides methods for evaluating the packaging of the viral particle by detecting the payload thereof. Existence of a higher amount of the payload indicates that the virus-producing cell replicates the viral vector robustly and therefore a higher chance that the produced viral particles are packaged with the viral vector.

Nucleic acid staining. In some embodiments, detecting a payload of the viral particle comprises introducing a nucleic acid dye into the microfluidic device and detecting a signal associated with the nucleic acid dye. In some embodiments, introducing the nucleic acid dye comprises diffusing the nucleic acid dye into the chamber. In some embodiments, the method further comprises, before detecting the signal associated with the nucleic acid dye, introducing a fluidic medium that does not comprise the nucleic acid dye into the flow region thereby removing the unbound nucleic acid dye. In some embodiments, the nucleic acid dye is a DNA dye or a RNA dye. In some embodiments, the nucleic acid dye is a fluorescent DNA dye, including but not limited to SYTO Green, QuantiFluor, GelGreen, or SYBR Gold.

In some embodiments, the method further comprises de-stabilizing the viral particle before introducing the nucleic acid dye. De-stabilizing the viral particle can facilitate the entry of the nucleic acid into the viral particle and/or release the viral vector from the viral particle and thereby increasing the interaction between the nucleic acid dye and the viral vector. In some embodiments, de-stabilizing the viral particle is performed by heating, changing pH of the fluid contained in the chamber, or a combination thereof.

In certain embodiments, heating comprises raising the temperature of the culture environment of the microfluidic device to about 30, 35, 40, 45, 50, 55, or 60 C degrees, or any range formed by two of the foregoing end points. In certain embodiments, the temperature is raised locally by projecting a laser beam at a selected area of the microfluidic device, for example, within an area of the flow region that is adjacent to the chamber or an area within the chamber.

In some embodiments, changing pH comprises adjusting the pH of the fluidic environment of the chamber to pH 6.5, 6.3, 6.0, 5.7, 5.5, 5.3, or 5, or any range formed by two of the foregoing end points. In certain embodiments, the pH is adjusted by introducing a buffer of desired pH into the flow region to replace the fluid originally in the chamber.

In some embodiments, the method further comprises, before de-stabilizing the viral particle, introducing a nuclease into the microfluidic device. In certain embodiments, the nuclease comprises DNase I, benzonase, micrococcal nuclease, a restriction endonuclease, or combinations thereof. Without being bound by theory, the nuclease degrades the nucleic acids of the cells that can otherwise be stained by the nucleic acid dye resulting in background noise in detection. In some embodiments, the method further comprises introducing a fluidic medium that does not comprise the nuclease into the flow region thereby removing remaining nuclease from the microfluidic device and stopping the degradation before de-stabilization.

Payload amplification and detection. Without being bound by theory, amplifying the payload can facilitate the detection thereof. As used here, “amplifying the payload” refers to amplifying a portion of the payload or the entity thereof. In some embodiments, amplifying the payload comprises introducing a polymerase, a primer recognizing the payload (optionally, a set of primers including a forward primer and a reverse primer having sequences complementary to a sequence of the payload), and dNTPs into the microfluidic device. In some embodiments, amplifying the payload comprises performing an isothermal amplification. In some embodiments of performing an isothermal amplification, amplifying the payload further comprises introducing a recombinase, and optionally a crowding reagent including but not limited to polyethylene glycol (PEG).

It is important to note that, in some embodiments, the amplification of payload (e.g., the isothermal amplification) is conducted in the presence of the virus-producing cells or debris thereof. It is also important to note that, in some embodiments, the amplification of payload (e.g., the isothermal amplification) is conducted within a chamber of a microfluidic device, especially a microfluidic device as described herein. Performing the amplification in a microenvironment (e.g., at a scale of nanoliter) in the presence of cells or debris thereof can be challenging because the cells, debris thereof, and molecules/proteins secreted therefrom would hinder the reaction. In some circumstances, they might react with the reagent for amplification and form clusters, which can impede flow or diffusion within the microfluidic device and/or affect the amplification or any subsequent assays. In a microenvironment such as a microfluidic device described therein, the clusters can significantly impede the diffusion of reagents into the chamber and the efficiency of the amplification.

Therefore, without being bound by theories, in certain embodiments, the crowding reagent is introduced separately from the polymerase and/or the primer. For example, a fluidic medium comprising a mixture having a polymerase and a primer set is introduced and diffuses into the chamber for a period of time. Then, a fluidic medium (or a buffer for performing the isothermal amplification) comprising a crowding reagent is introduced into the flow region for a period of time to replace the previous fluidic medium and let the crowding reagent diffuse into the chamber.

In some embodiments, the isothermal amplification is initiated by an initiator, such as Magnetism (MgOAc). In such embodiments, an initiator is introduced after a recombinase, a polymerase, a pair of primer, dNTP, and a crowding reagent are introduced and present in the chamber.

In some embodiments, the amplified payload can be detected by introducing a nucleic acid dye into the flow region and detecting a signal associated with the nucleic acid dye. The nucleic acid dye can be as described herein. Alternatively, the amplified payload can be detected using a probe. In some embodiments, the probe can comprise a detectable label and a complementary sequence configured to hybridize the payload. In such embodiments, the amplified payload can be detected by detecting a signal associated with the detectable label. In some embodiments, the complementary sequence is configured to hybridize a portion or an entirety of the payload.

In certain embodiments, detecting the payload comprises introducing an endonuclease and a first probe configured to recognize the payload, wherein the first probe comprises a detectable label and a quencher moiety configured to quench the detectable label; and binding the first probe to the payload thereby forming a probe: payload complex; cleaving the first probe of the probe: payload complex with the endonuclease thereby releasing the detectable label from the probe: payload complex; and detecting a signal associated with the detectable label thereby detecting the payload of the viral particle.

In some embodiments, detecting the payload of the viral particle comprises introducing an endonuclease: nucleic acid complex and a second probe comprising a detectable label and a quencher configured to quench the second detectable label, wherein the nucleic acid of the endonuclease: nucleic acid complex has a complementary sequence configured to hybridize the payload; binding the endonuclease: nucleic acid complex to the payload thereby activating the endonuclease of the endonuclease: nucleic acid complex; and cleaving the detectable label from the second probe by the activated endonuclease, and detecting a signal associated with the detectable label. In some embodiments, the complementary sequence is configured to hybridize a portion or an entirety of the payload.

In certain embodiments, the endonuclease: nucleic acid complex comprises a nucleic acid comprising a first segment complementary to the payload and a second segment comprising a clustered regularly interspaced short palindromic repeats (CRISPR) sequence. As used herein, “complementary to the payload” refers to complementary to a portion or an entirety of the payload. The endonuclease can be activated upon recognizing or binding the payload by the endonuclease: nucleic acid complex through the hybridization between the nucleic acid and the payload.

In some embodiments, the endonuclease: nucleic acid complex is prepared by incubating an endonuclease with the nucleic acid for a period of time. In some embodiments, the endonuclease: nucleic acid complex is a mixture of various kind of endonuclease: nucleic acid complexes. Each kind of the endonuclease: nucleic acid complexes can comprise a single kind of endonuclease but a different kind of nucleic acid. Each kind of nucleic acid comprises a sequence that is complementary to the payload. In some embodiments, the endonuclease: nucleic acid complex is a mixture of three, five, or ten kinds of endonuclease: nucleic acid complexes, and each endonuclease: nucleic acid complex comprises a respective sequence complementary to the payload.

The activated endonuclease can cleave the second probe thereby releasing the detectable label from the quencher. In certain embodiments, the endonuclease can be a CRISPR-associated (Cas) protein, including but not limited to Cas-12a, Cas-13, Cas-13a, Csm6, or a mixture thereof. In certain embodiments, detecting the payload of the viral particle comprises performing a CRISPR-based DETECTR assay or a CRISPR-based SHERLOCK assay.

It is important to note that, in some embodiments, the detecting is performed in the presence of the virus-producing cells or debris thereof. It is also important to note that, in some embodiments, the detecting is performed within a chamber of a microfluidic device, especially a microfluidic device as described herein. The microenvironment poses uncertainty and challenges as a conventional DETECTR or SHERLOCK assay or other CRISPR based detection assay is performed with a sample of purified genetic materials in a PCR tube.

In some embodiments, the endonuclease: nucleic acid complex is introduced earlier than the second probe. Without being bound by theory, an endonuclease: nucleic acid complex is normally larger than a probe. Therefore, by introducing the endonuclease: nucleic acid complex into the flow region earlier than the probe ensures the endonuclease: nucleic acid complex has sufficiently diffused into the chamber when probe is introduced and diffuses. That mitigates the risk of probe being cleaved non-specifically by other nucleases that might be present in the chamber so that background noise of the detection can be reduced. In certain embodiments, a first fluidic medium comprising the endonuclease: nucleic acid complex is introduced into the flow region of the microfluidic device, and then a second fluidic medium comprising the second probe is introduced into the flow region. In some embodiments, the second fluidic medium comprises the endonuclease: nucleic acid complex and the second probe.

In some embodiments, as there might be endogenous nucleases secreted by the virus-producing cell in the chamber that can cleave the second probe thereby resulting in background noise of the detection. The background noise caused by the endogenous nucleases can be further mitigated by normalizing the detected signal with a pre-assay signal, treating the chamber with a nuclease inhibitor, using a suitable second probe, or a combination thereof according to the present disclosure.

Normalization with pre-assay signal. In some embodiments, before introducing the endonuclease: nucleic acid complex, a third fluidic medium comprising the second probe and not comprising the endonuclease: nucleic acid complex can be introduced into the flow region, and an image can be taken to detect a pre-assay signal associated with the detectable label of the second probe. Because the endonuclease: nucleic acid complex for performing the assay does not yet exist in the microfluidic device, the pre-assay signal represents the background noise generated by the endogenous nucleases. The background noise can therefore be mitigated by normalizing a post-assay signal detected after introducing the endonuclease: nucleic acid complex with the pre-assay signal.

Nuclease inhibitor. In some embodiments, the background noise can be mitigated by treating the chamber with a nuclease inhibitor. In certain embodiments, the nuclease inhibitor is a proteinase, including but not limited to trypsin.

Design of the second probe. In some embodiments, the background noise can be mitigated by selecting a suitable probe. In some embodiments, the second probe is an adenine (A)/thymine (T) rich probe, which is suitable for Cas-12a protein. In certain embodiments, the A/T rich probe comprises adenine or thymine at more than 50%, 55%, 60%, 65%, 70%, 75%, 80%, 85%, 90%, 95%, 99%, or about 100% or any range formed by two of the foregoing end points of its sequence. Alternatively, without being bound by theory, the endogenous nuclease leading to background noise may exhibit bias toward the composition of the second probe. A probe composition that is less favor for the endogenous nuclease can reduce the background noise of the detection. Therefore, in some embodiments, the second probe is a cytosine (C) rich probe. In certain embodiments, the C rich probe comprises cytosine at more than 50%, 55%, 60%, 65%, 70%, 75%, 80%, 85%, 90%, 95%, 99%, or about 100% or any range formed by two of the foregoing end points of its sequence. In some embodiments, the second probe can be modified with phosphorothioate bonds (PS bond) at 3′ and/or 5′ end thereof.

Detectable label. The detectable label described herein may be a visible, luminescent, phosphorescent, or fluorescent detectable label. In some embodiments, the detectable label may be covalently attached directly or indirectly to the binding component of the reporter molecule, or the probe described herein. In certain embodiments, the detectable label is a fluorophore.

Capping. In some embodiments, evaluating a productivity of the virus-producing cell further comprises capping the chamber thereby substantially isolating the chamber from the flow region. Without being bound by theory, given the small size of the viral particle, the viral particle produced can diffuse out of the chamber relatively quickly during assaying, therefore, the capping can retain the viral particle within the chamber to prevent from cross-contamination between chambers of the microfluidic device.

In some embodiments, the capping comprises introducing a water immiscible fluidic medium into the flow region of the microfluidic device. In certain embodiments, the water immiscible fluidic medium is an alkane, a fluoroalkane, an oil, a hydrophobic polymer, or any combination thereof. In some embodiments, the oil is a fluorinated oil, including but not limited to Hydrofluoroether (Novec 7500 Engineered Fluid), 2-(Trifluoromethyl)-3-ethoxydodecafluorohexane (HFE-7500, 3M™, Novec™), Fluorinert™ FC-40, (Aldrich Cat No. F9755), Fluorinert™ FC-70, (Aldrich Cat No. F9880), (Tridecafluoro-1,1,2,2,-tetrahydrooctyl) tetramethyldisiloxane (Gelest, Cat #SIB1816.0). Capping using a water immiscible fluidic medium is described in WO2020223555, filed on Apr. 30, 2020, which is incorporated herein by reference. In some embodiments, the capping comprises introducing air to replace the liquid in the flow region.

### Split Clone

When a virus-producing cell or clonal populations thereof with preferred productivity is identified, there might be a need to export the identified virus-producing cells or clonal populations thereof. However, the viability of the virus-producing cells could be compromised during the assaying for evaluating the productivity. Therefore, in another aspect, the present disclosure provides methods for preserving a subset of biological micro-objects within a microfluidic device. That methods of the present disclosure offer the advantages to culture, expand, and evaluate the viral particle production of virus-producing cells while preserve a subset of the clonal population in the microfluidic device. In this way, after the evaluation, the preserved subset of cells, which is of the single clonality of the cells being evaluated, can be exported and collected for subsequent analysis and applications.

The method for preserving a subset of biological micro-objects within a microfluidic device comprises: providing a microfluidic device, wherein the microfluidic device comprises a microfluidic circuit material defining a flow region and a plurality of chambers fluidically connecting to the flow region, and further wherein the plurality of chambers comprises a first chamber and a second chamber, and further wherein a plurality of biological micro-objects is disposed within the first chamber; moving a first subset of the plurality of biological micro-objects into the second chamber; designating one of the first chamber and the second chamber as a preserving chamber and the other as an assay chamber; and forming a first in situ-generated cap within the preserving chamber, wherein the first in situ-generated cap comprises a porosity to selectively block passage between the preserving chamber and the flow region.

As used herein, a “subset” of a clonal population can comprise 1, 2, 3, 4, 5, 6, 7, 8, 9, 10, or more cells of the clonal population. As used therein, “preserve a subset of the clonal population” refers to isolating the subset from the rest of the clonal population so that the cell of the subset would not be substantively or significantly affected by an assay performed to the rest of the clonal population.

As used herein, “selectively block” refers that the porosity of the in situ-generated cap can block some molecules or micro-objects based on the size thereof. In other words, some molecules or objects can pass through the in situ-generated cap if they are small enough. In some embodiments, the in situ-generated cap blocks the passage of the cells and the reagents required for performing an assay in the assay chamber.

As used herein, a “preserving chamber” refers to a chamber that is isolated from an assay performed in the assay chamber. Nevertheless, it does not necessarily mean that a cell in the preserving chamber will not be assayed at all. In other words, a cell in the preserving chamber can later be assayed if needed. As used herein, an “assay chamber” refers to a chamber where an assay is performed while the cell within the preserving chamber is isolated or preserved from being affected by the assay.

In some embodiments, the methods of the present disclosure are performed on a microfluidic device as described herein. The microfluidic device comprises a substrate comprising an optically-actuated DEP electrode activation and a microfluidic circuit comprising a flow region and a plurality of chambers. In some embodiments, moving the first subset of the plurality of biological micro-objects into the second chamber comprises using dielectrophoresis (DEP) force, gravity, or a combination thereof.

Two or more of the chambers can be grouped as a culture unit for a clonal population of virus-producing cells. Among the culture unit, at least one of the chambers can be designated as a preserving chamber and at least one of the chambers can be an assay chamber.

In some embodiments, two chambers are grouped as a culture unit. One of the chambers of the culture unit can be designated as a preserving chamber and the other is as an assay chamber. In some embodiments, three chambers are grouped as a culture unit. One of the chambers of the culture unit can be designated as a preserving chamber and the other two chambers are used as assay chambers. In such embodiments, different assays can be performed in the two assay chambers respectively provided that the assay chambers are also isolated from each other using an in situ-generated cap. In yet other embodiments, a plurality of chambers is grouped as a culture unit. More than one of the chambers are used as preserving chambers and the rest chambers are used as assay chambers for various assays if needed.

In some embodiments, providing the microfluidic device further comprising disposing a biological micro-object into the first chamber, and expanding the biological micro-object into the plurality of biological micro-objects. In some embodiments, disposing a biological micro-object into the first chamber comprises introducing a fluidic medium comprising the biological micro-object into the flow region and disposing the biological micro-object into the first chamber. In some embodiments, expanding the biological micro-object into the plurality of biological micro-objects comprises perfusing a culture medium into the flow region and allow the biological micro-object to growth and proliferate within the chamber.

In some embodiments, moving the first subset of the plurality of biological micro-objects into the second chamber comprises: selecting a biological micro-object from the plurality of biological micro-objects in the first chamber; moving the selected biological micro-object from the first chamber into the second chamber thereby forming a first subset of the plurality of biological micro-objects in the second chamber.

In some embodiments, moving the first subset of the plurality of biological micro-objects into the second chamber comprises: moving a biological micro-object from the first chamber into a transit area within the flow region and from the transit area into the second chamber thereby forming the first subset of the plurality of biological micro-objects in the second chamber.

As illustrated in FIGS. 7A-7E, a virus-producing cell 701 introduced into the microfluidic channel of the microfluidic device having a plurality of chambers (only a pair of the chambers are shown in the figure). The cell can be disposed into a chamber 711 (FIG. 7A), which together with chamber 712 are grouped as a culture unit 720. One of the chambers 711 and the chamber 712 will be used as an assay chamber and the other one will be the preserving chamber. The cell is maintained in the assay chamber and can be moved from the chamber 711 to the chamber 712 at any time depending on the needs of the experiments. In some embodiments, before being moved, the cell 701 expands into a clonal population in the chamber 711 (FIG. 7B).

In some embodiments, the cells are adhesive to the surface of the chamber. Therefore, before being moved, the cells are treated with proteinase thereby being dissociated from the surface (FIG. 7C). The reagent or proteinase used for the dissociation is not limited. In some embodiments, TrypLE™ is used for dissociation purposes. In some embodiments, EDTA is additionally added to facilitate the dissociation. In some embodiments, bubble dislodge using laser can be applied to facilitate the dissociation. Bubble dislodge can be as described in WO2017117408, filed on Dec. 29, 2016, and WO2020168258, filed on Feb. 14, 2020, the contents of which are incorporated herein by reference. In some embodiments, if the cells cultured in the chamber are not adherent cells, the dissociation might not be needed. Then, a subset of the clonal population is moved to the chamber 712 while the rest of the clonal population is retained in the chamber 711 (FIG. 7D).

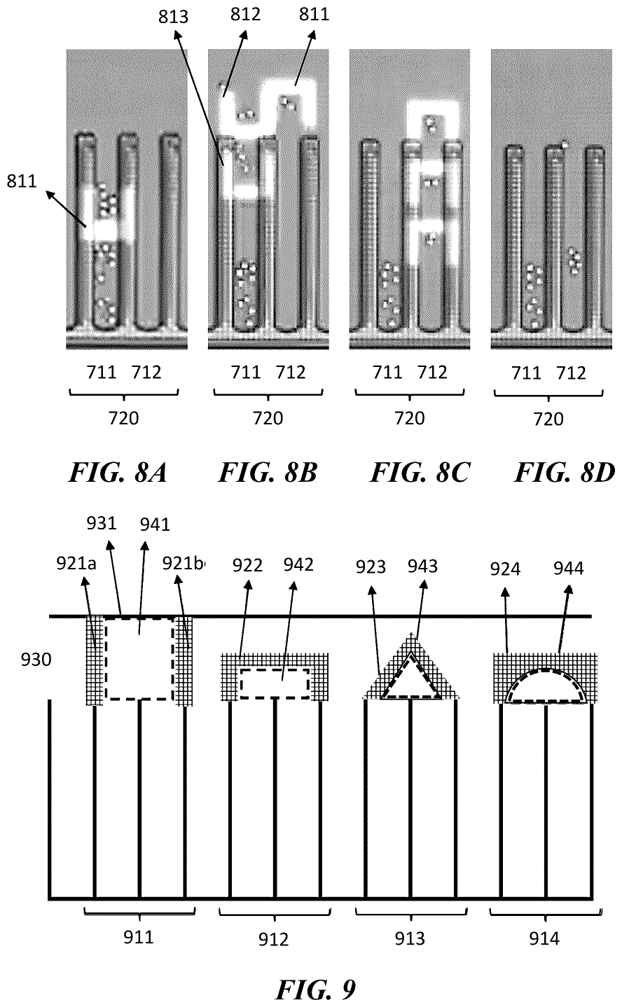

Active split. In some embodiments, “moving” as used here comprises moving the first subset actively comprising using DEP force, gravity, or centrifugation. In some embodiments of active moving, the subset is moved using DEP force as illustrated in FIGS. 8A-8D. In FIG. 8A, a pattern of the DEP electrode regions was illuminated and thereby activating local DEP forces that attract or repel nearby micro-objects (in the example shown in FIGS. 8A-8D, the local DEP forces repel the cells). In this embodiment, the pattern of the activated DEP electrode regions forms an open square light cage 811 surrounding the cells to be moved; while in other embodiments, the pattern can form a single light bar or a light cage of different shape. The open square light cage 811 can be moved by moving the light pattern and thereby moving the cells. More description regarding moving cells using DEP force is described in WO2016094459, filed on Dec. 8, 2015, the contents of which are incorporated herein by reference.

In FIG. 8B and FIG. 8C, a light sequence creating more than one light cage (light cage 812 and light cage 813) is applied to facilitate the movement of the cells. As shown in the figures, cells were moved from the first chamber to a transit area within the flow region and then moved to the second chamber. More description regarding moving cells using conveyor light sequence is described in U.S. Pat. No. 10,675,625, filed on Apr. 14, 2017, the contents of which are incorporated herein by reference. As a result, a subpopulation of the clonal population is moved into the chamber 712 and the rest is kept in the chamber 711 as shown in FIG. 7D and FIG. 8D.

In some embodiments, a transit area is defined in a region of the flow region (e.g., the microfluidic channel) adjacent to the openings of the chambers of the culture unit. The transit area can be substantially enclosed by an in situ-generated barrier thereby preventing the biological micro-object from entering an area of the flow region other than the transit area while allowing the micro-object to move or to be moved between the transit area, the first chamber, and the second chamber. While the transit area can be fluidically connected to the chambers of the culture unit, it is substantially isolated from other regions of the microfluidic channel and from other chambers of the microfluidic device. Cells in other chambers or regions of the microfluidic channel are blocked from entering the transit area of the culture unit, and cells within the transit area and the culture unit are not allowed to leave.

In most embodiments, it is not limited whether substances other than the cells to be moved are restricted to enter or leave the transit area. In some embodiments, the fluid within the microfluidic device is not blocked to enter or leave the transit area.

FIG. 9 illustrates some examples of the transit area (the area within the dotted line) formed in the microfluidic channel. The culture unit 911 comprises two chambers. Two in situ-generated barriers 921a and 921b are formed from the openings of the chambers to the wall 931 of the microfluidic channel 930. A transit area 941 is defined by the wall 931 and the two in situ-generated barriers 921a and 921b. For culture unit 912, an in situ-generated barrier of open square shape 922 is formed to define a transit area 942. For culture unit 913, a in situ-generated barrier of open arrow shape is 923 is formed to define a transit area 943 of triangle shape. Lastly, for culture unit 914, an in situ-generated barrier 924 of arch bridge is formed to define a transit area 944. The shape of the in situ-generated barrier and the shape of the transit area are not so limited.

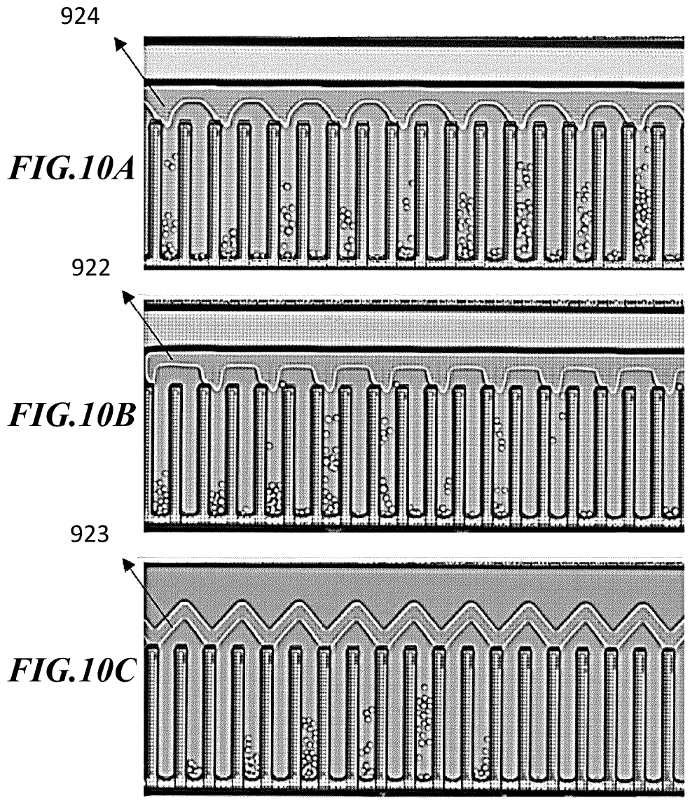

FIG. 10A shows an example of the in situ-generated barrier 924 and the transit area 944. FIG. 10B shows an example of the in situ-generated barrier 922 and the transit area 942. FIG. 10C shows an example of the in situ-generated barrier 923 and the transit area 943.

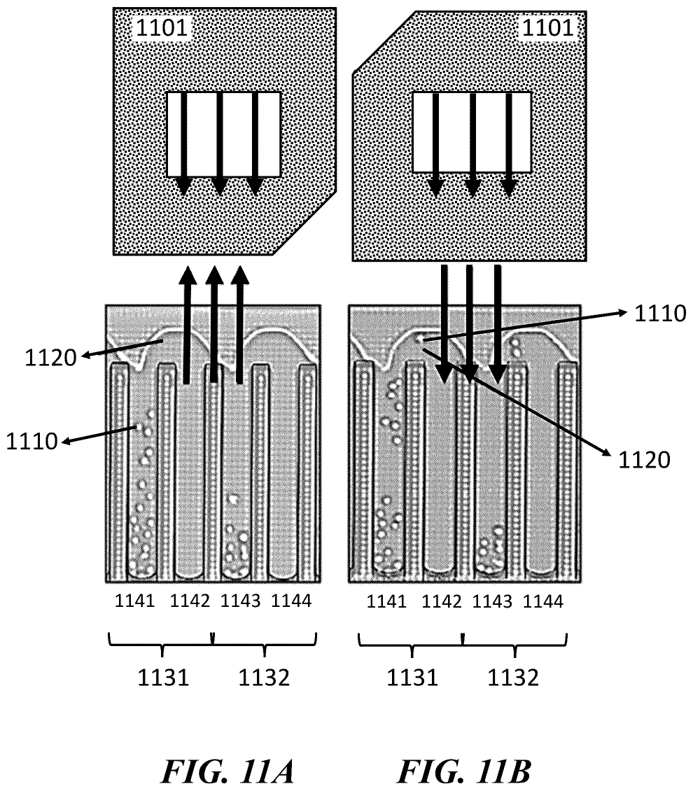

After the transit area is formed, the cells can be moved by DEP force or by gravity by tilting the microfluidic device from a certain angle to let the cells drop into the transit area. For example, in the embodiment showed in FIG. 11A, two culture units 1131 and 1132, each comprising a first chamber 1141, 1143 and a second chamber 1142. 1144 were shown. The microfluidic device 1101 was put under centrifugation to generate a force (to the arrowed direction) pushing cells 1110 to drop into the transit area 1120; or the microfluidic device 1101 was tilted upside down to let the cells 1110 drop into the transit area 1120. In some embodiments, all cells originally in the first chamber 1141, 1143 dropped to the transit area, while in some embodiments, only a portion of the cells dropped to the transit area.

Then, another centrifugation was applied to generate a force in opposite direction or the microfluidic device 1101 was tilted from the opposite side so that the cells dropped from the transit area 1120 (FIG. 11B) back to the culture unit 1131, 1132. This time, some cells dropped into the original chamber (the first chamber 1141, 1143), and some dropped into the second chamber 1142, 1144 of the culture unit.

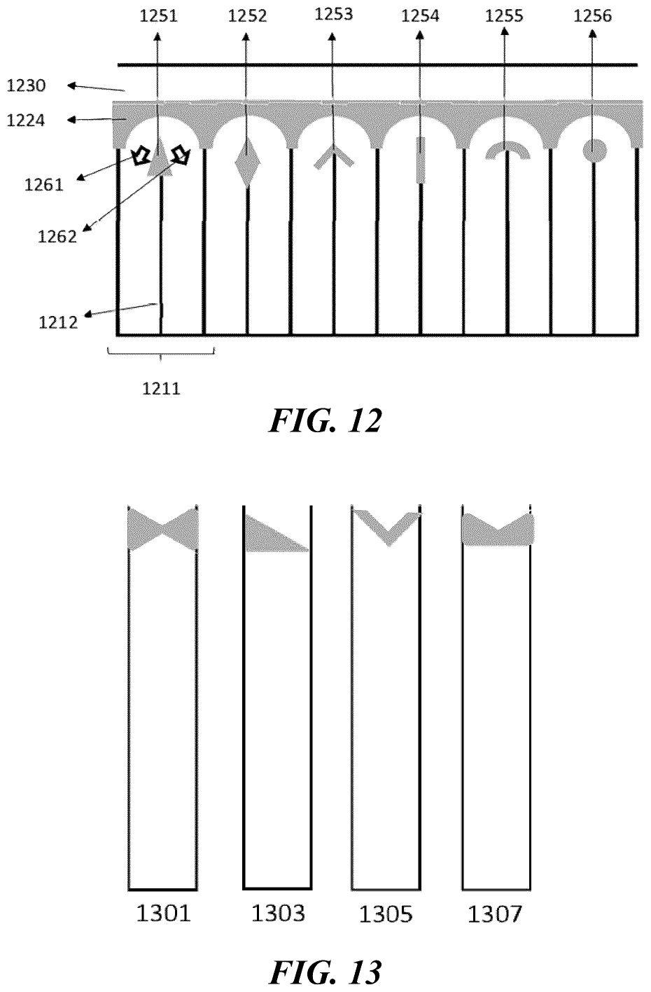

In some embodiments, the method further comprises forming an in situ-generated separating element at an area between the openings of the chambers of a culture unit. As shown in FIG. 12, a separating element is formed at the tip of a shared wall 1212 of the two chambers of a culture unit 1211 to facilitate the process of moving cells from a transit area back to the chambers. For example, the separating element can be a triangular separating element 1251. When cells are collected in the transit area defined by in situ-generated barrier 1224 formed in-situ within a microfluidic channel 1230 and then dropped back to the culture unit 1211 by gravity, cells will be divided by the triangular separating element 1251 and fall in the direction guided by the separating element 1251 (See the two directions indicated by arrow 1261 and arrow 1262). The in situ-generated barrier 1224 has a similar shape as the in situ-generated barrier 924 shown in FIG. 9 and FIG. 10A.

Please note that cells are not shown in FIG. 12 for the convenience to see the relation between the separating element, the culture unit, and the in situ-generated barrier defining the transit area. In some embodiments, the separating element is an in situ-generated separating element. In some other embodiments shown in FIG. 12, the separating element can be a diamond shape (separating element 1252), an arrow (separating element 1253), a vertical bar (separating element 1254), a curved bar (separating element 1255), or a round shape (separating element 1256). The shape of the separating element is not limited as long as it can facilitate in separating cells into the assay chamber and the preserving chamber.

Turning back to FIG. 7D, in some embodiments, moving a first subset of the plurality of biological micro-objects into the second chamber further comprises retaining a second subset of the plurality of biological micro-objects in the first chamber. In some embodiments, after the cells are split, the cells are cultured in both chambers of the culture unit. In some embodiments, the cells in the chambers of the culture unit and the preserving chamber are cultured under substantially the same conditions. As a result, the cells or population derived therefrom in the first chamber and the second chamber are of a single clonal population.

One of the chambers can be designated as an assay chamber and the other one can be designated as a preserving chamber as described above. The preserving chamber (in the embodiment of FIG. 7E) is capped by an in situ-generated cap 730 thereby separating or isolating the preserving chamber from the assay chamber and the microfluidic channel. However, separating or isolating the preserving chamber from the assay chamber and the microfluidic channel does not necessarily mean all fluidic communication between the preserving chamber and the microfluidic channel is blocked. As described above, the in situ-generated cap is made of a hydrogel having a porosity that does not allow the reagents used for the assay and/or the products resulted from the assay to pass through. The porosity, however, allows nutrition and waste to pass through so that the cells within the preserving chamber can still be maintained in good condition.

In some embodiments, the in situ-generated cap is formed within the preserving chamber. In some embodiments, the in situ-generated cap is formed within an area within the chamber and close to the opening of the chamber to the flow region.

In some embodiments, the in situ-generated cap is a hydrogel cap. In some embodiments, the in situ-generated cap is made of reversible hydrogel as described herein so that the hydrogel cap can be dissolved for exporting cells within the preserving chamber. In some embodiments, the hydrogel cap is moveably connected to one or more surface of the preserving chamber. As used here, “moveably connected” describes that the hydrogel cap can be moved upon a threshold pressure. Therefore, applying a threshold pressure to the hydrogel cap moves with respect to the one or more surfaces of the chamber and thereby creates an opening in the preserving chamber. The opening can facilitate the exportation of the cell out of the preserving chamber.

It may be desired to select a hydrogel cap design that provides non-uniformity in a center point of the width of the chamber, decreasing the width, thickness or height of the hydrogel gels/segments as the forces created by the laser illumination are directed more forcefully there. In some embodiments, the hydrogel cap comprises a non-uniform thickness with respect to an axis of the chamber such that a portion of the in situ-generated barrier is less thick than other portions of the hydrogel cap. In some embodiments, the less thick portion of the hydrogel cap has a thickness that is smaller than the height of the chamber.

Some examples of the shapes of the hydrogel caps in this aspect are shown in FIG. 13. They include a bowtie shape cap 1301 (a cap formed by two triangular bars joining together at the apexes thereof so that the whole structure has a tapering thickness at the center), a single triangular bar 1303, a V-shaped bar 1305, and a V-cap 1307 (a rectangular bar having a v shaped depression on the side of the barrier facing the opening to the channel).

In some embodiments, the assay chamber is used to perform an assay as described herein including but not limited to inducing the production of viral particles, evaluating physical titer, and/or evaluating genetic titer. For example, if the first chamber is used as the assay chamber, the second subset of the plurality of biological micro-objects retained in the first chamber is allowed or induced to produce a biological product of interest. Conversely, if the second chamber is used as the assay chamber, the first subset of the plurality of biological micro-objects retained in the second chamber is allowed or induced to produce a biological product of interest. In either way, the biological micro-objects preserved in the preserving chamber are protected from being affected by the assays performed in the assay chamber. Preferably, the assay can be performed after the in situ-generated cap is formed to seal the preserving chamber.

In some embodiments, performing an assay in the assay chamber comprises further comprises assaying the biological product of interest. In many embodiments, the biological product is produced by the biological micro-object disposed within the assay chamber. The biological product can be secreted by the biological micro-object, presented on the surface of the biological micro-object, and/or retained within the biological micro-object. As used here, “assaying” the biological product of interest comprises performing an experiment directed to the biological product in order to characterize, quantify, evaluate a property of the biological product. The biological product of interest can be a protein, a molecule, or a biological micro-object. In certain embodiments, the biological micro-object is a virus-producing cell, and the biological product of interest is a viral particle.

In some embodiments, performing an assay in the assay chamber comprises a lysis step comprising introducing a lysis buffer or a lysis solution into the flow region of the microfluidic device; diffusing the lysis buffer into the assay chamber while blocking the lysis buffer from entering the preserving chamber with the in situ-generated cap. The lysis step can be performed to release a biological product from the biological micro-object. In some embodiments, the lysis step is performed to assess the genomic information of the biological micro-object, for example, to assess a mRNA barcode as described herein to verify the identity of the biological micro-object. Methods of on chip cell lysis, mRNA capture, and cDNA library generation have been described, for example, in PCT International Application No. WO2018064640, filed Sep. 29, 2017, the entire contents of which is incorporated herein by reference.

### Exportation and Clonality

After a cell of interest (e.g., a cell of desired productivity) is identified, the cell of interest can be exported for further analysis or uses. In some embodiments, a clonal population is identified as having the desired productivity so that a subset of the clonal population can be exported. In some embodiments, exporting a cell of interest comprises dislodging the cell of interest from a chamber to the flow region (e.g., a microfluidic channel) and applying a flow into the flow region to export the cell of interest out of the microfluidic device. The exported cell of interest can then be collected for further analysis or uses.

In some embodiments, a clonal population is disposed in a first chamber and split as described herein resulting in a first subset of the clonal population disposed in a second chamber and a second subset of the clonal population retained in the first chamber. The first chamber is designated as an assay chamber, and an assay is performed to evaluate the productivity of the clonal population. The second chamber is designated as a preserving chamber, and an in situ-generated cap is formed within the preserving chamber to protect the first subset of the clonal population from assay reagents. If an assay conducted in the first chamber identifies the clonal population to have desired productivity, a third subset of the clonal population from the preserved first subset in the second chamber can be exported. In some embodiments, the fourth subset of the clonal population can also be exported from the second subset in the first chamber for further verification.

In some embodiments, exporting a cell of interest comprises removing an in situ-generated cap formed with in the chamber where the cell of interest is disposed. Removing the in situ-generated cap can comprise dissolving or moving the in situ-generated cap as described herein so that it no long blocks the passage of the cells to be exported.

In some embodiments, in order to prevent from cross-contamination with other chambers within the microfluidic device during exportation, exporting a cell of interest comprises in-situ gelling an interior space of chambers that the cell therein is not to be exported. In some embodiments, exporting a cell of interest comprises in-situ gelling an interior space of the assay chamber. The gelation of the assay chamber prevents the remaining assay reagent from leaving the assay chamber and affect the exported cell of interest.

In some embodiments, the in-situ gelling comprises forming a second in situ-generated cap within the chamber that the cell therein is not to be exported. In some embodiments, the in-situ gelling comprises forming an in situ-generated structure occupying about 10%, 20%, 30%, 40%, 50%, 60%, 70%, 80%, 90%, 95%, 99%, or 100% of the interior space of the chamber that the cell therein is not to be exported, or any range formed by two of the foregoing end points. In some embodiments, before forming an in situ-generated structure occupying the interior space of the chamber, air is perfused to remove remaining liquid in the microfluidic channel.

Identifying a virus-producing cell of desired productivity from a plurality of virus-producing cells. In some embodiments, the microfluidic device comprises a plurality of chambers, and a plurality of virus-producing cells are introduced into the microfluidic device. Single virus-producing cells can be disposed into a respective chamber of the plurality for evaluating their productivities individually according to the methods of the present disclosure. Based on the evaluation, a virus-producing cell or clonal population therefrom among the plurality of virus-producing cells of desired productivity can be identified.

In an exemplary embodiment, the plurality of virus-producing cells comprises a first virus-producing cell and a second virus-producing cell. In such embodiments, evaluating a productivity of the virus-producing cell comprises: evaluating a first productivity of the first plurality of virus-producing cells and evaluating a second productivity of the second plurality of virus-producing cells; and selecting a cell of interest from the first plurality of virus-producing cells and the second plurality of virus-producing cells based on comparing the first productivity and the second productivity. In some embodiments, comparing the first productivity and the second productivity comprises comparing the first productivity and the second productivity with a threshold productivity. In some embodiments, comparing the first productivity and the second productivity comprises comparing the first productivity with the second productivity. In some embodiments, both the first plurality of virus-producing cells and the second plurality of virus-producing cells are selected as cells of interest.

In some embodiments, a plurality of virus-producing cells is introduced into a microfluidic device comprising a first chamber, a second chamber, a third chamber, and a fourth chamber. A first virus-producing cell of the plurality is disposed into the first chamber, the first chamber and the second chamber are grouped as a first culture unit for culturing and assaying the first virus-producing cell and clonal population therefrom, while a second virus-producing cell of the plurality is disposed into the third chamber, and the third chamber and the fourth chamber are grouped as a second culture unit for culturing and assaying the second virus-producing cell and clonal population therefrom.

The first virus-producing cell and the second virus-producing cell can expand into a first clonal population and a second clonal population in the first chamber and the third chamber respectively. A subset of the first clonal population is preserved into a second chamber, and a subset of the second clonal population is preserved into a fourth chamber according to the methods of the present disclosure. The second chamber and the fourth chamber can be designated as preserving chambers with in situ-generated caps formed therewithin, and the first chamber and the third chamber are designated as assay chambers.

In some embodiments, one or both of the first virus-producing cell and the second virus-producing cell can be selected by evaluating the productivities thereof based on the methods of the present disclosure. The cells of the preserving chambers of a selected clonal population can be exported for further analysis or uses, including but not limited to quantitative polymerase chain reaction for verifying the payload carried by viral particle or off-chip culture for manufacturing viral vector-dependent products.

In some embodiments, exporting the selected cells of interest comprises identifying a selected chamber wherein the selected virus-producing cell of interest is located; forming a third in situ-generated cap within a chamber of the microfluidic device except for the selected chamber; and dislodging the selected virus-producing cell of interest from the selected chamber into the flow region.

In some embodiments, the virus-producing cell comprises a mRNA barcode presenting the identity of the virus-producing cells. In such embodiments, the mRNA barcode can be collected for identifying the selected virus-producing cell. In some embodiments, the method further comprises disposing a mRNA capture bead configured to capture the mRNA barcode into the chamber and optionally performing reverse transcription. The mRNA capture bead and reverser transcription performed in the chamber is described in WO2022051570, filed on Sep. 3, 2021, which is incorporated herein by reference.

In some embodiments, the method further comprises lysing the virus-producing cell thereby releasing the mRNA thereof. In certain embodiments, lysing the virus-producing cell comprises introducing a lysis solution into the flow region and diffusing the lysis solution into the chamber comprising the selected virus-producing cell and the mRNA capture bead. In some embodiments, the mRNA capture bead can be exported out of the chamber, optionally, out of the microfluidic device for further verification.

In some embodiments, after lysing the virus-producing cell, the method further comprises removing the lysis solution out of the flow region, optionally by perfusing the microfluidic device with a viscosity gradient flush. In some embodiments, the viscosity gradient flush comprises perfusing the microfluidic device with post-lysis buffers of a gradient of viscosity. In certain embodiments, the viscosity gradient flush comprises perfusing a first post-lysis buffer of high viscosity followed by a second post-lysis buffer of low viscosity. In certain embodiments, the first post lysis buffer comprises about 10, 11, 12, 13, 14, 15, 16, 17, 18, 19, or 20% glycerol, or any range formed by two of the foregoing end points. In some embodiments, the second post lysis buffer comprises about 5, 6, 7, 8, 9, or 10% glycerol, or any range formed by two of the foregoing end points, glycerol.

### In Situ-Generated Structure

The term “in situ-generated structure” refers to a structure that is formed in a selected area while the microfluidic device is in operation. The barrier is generally not formed while manufacturing the microfluidic device or does not exist before the microfluidic device is used for experiments or research. The term “structure” refers to a physical structure that is formed and fixed, at least for a certain period of time, in a selected area and is capable of impeding or blocking a particle (e.g., a micro-object or a molecule) from crossing through the structure.

The in situ-generated structure of the present disclosure includes but not limited to the in situ-generated cap, in situ-generated barrier, or in situ-generated separating element. Generally, one of the functions of the in situ-generated structure in the methods of the present disclosure is to contain a biological micro-object (e.g., a cell or a viral particle) within an area of the microfluidic device or to guide movement of a biological micro-object toward an area of the microfluidic device. In some embodiments, the structure defines an enclosed area within the chamber or within the flow region. The in situ-generated structure of the present disclosure can also include the in situ-generated capture structure, which provides not only a physical barrier, but also functionalized sites configured to bind a micro-object of interest.

In some embodiments, the impediment or block produced by introduction of the structure is size-dependent. A particle can be impeded, blocked, or allowed to cross through the barrier depending upon its size. In some embodiments, the in situ-generated structure has a porosity that substantially prevents a cell or a viral particle from crossing through the in situ-generated structure. In some embodiments, the in situ-generated structure has a porosity that substantially prevents an assay reagent from crossing through the in situ-generated structure. For example, an in situ-generated cap sealing a preserving chamber of a culture unit as described above can prevent an assay reagent required for entering the preserving chamber so that the assay can only be performed in an unsealed chamber, which in this embodiment is the assay chamber of the culture unit. The assay reagent that is blocked from entering the preserving chamber can be any reagent required for performing an assay in the assay chamber and can also include the AdV required to induce the production of AAV particles in the assay chamber.

In another aspect, an in situ-generated cap may be additionally used to reduce clonality risk. As described above, the cells kept in the preserving chamber can be exported after being identified as exhibiting desired productivity. While exporting the desired cells or clonal populations thereof from the preserving chamber, and optionally from the microfluidic device, in order to prevent loss of clonality, another in situ-generated cap can be formed to seal the openings of undesired/unselected chambers, reducing the risk that cells that do not belong to the selected clonal population will be unpenned and mix with the cells selected for export.

Hydrogel composition. In certain embodiments, the in situ-generated structure is a hydrogel. In certain embodiments, the in situ-generated barrier comprises a solidified polymer network. In some embodiments, the solidified polymer network comprises a synthetic polymer, a modified synthetic polymer, or a biological polymer. In certain embodiments, the solidified polymer network comprises at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. In some embodiments, the solidified polymer network does not include a silicone polymer.

Physical and chemical characteristics determining suitability of a polymer for use in the solidified polymer network may include molecular weight, hydrophobicity, solubility, rate of diffusion, viscosity (e.g., of the medium), excitation and/or emission range (e.g., of fluorescent reagents immobilized therein), known background fluorescence, characteristics influencing polymerization, and pore size of a solidified polymer network. The solidified polymer network is formed upon polymerization or thermal gelling of a flowable polymer solution containing at least one of a polyethylene glycol, modified polyethylene glycol, polyglycolic acid (PGA), modified polyglycolic acid, polyacrylamide (PAM), modified polyacrylamide, poly-N-isopropylacrylamide (PNIPAm), modified poly-N-isopropylacrylamide, polyvinyl alcohol (PVA), modified polyvinyl alcohol, polyacrylic acid (PAA), modified polyacrylic acid, fibronectin, modified fibronectin, collagen, modified collagen, laminin, modified laminin, polysaccharide, modified polysaccharide, or a co-polymer in any combination. Various co-polymer classes may be used, including but not limited to any of the above listed polymers, or biological polymers such as fibronectin, collagen or laminin. Polysaccharides such as dextran or modified collagens may be used. The flowable polymer may be referred alternatively here as a pre-polymer, in the sense that the flowable polymer is crosslinked in-situ. Biological polymers having photoactivatable functionalities for polymerization may also be used.

In some instances, a polymer may include a cleavage motif. A cleavage motif may include a peptide sequence inserted into the polymer that is a substrate for one or more proteases, including but not limited to a matrix metalloproteinase, a collagenase, or a serine proteinase such as Proteinase K. Another category of cleavage motif may include a photocleavable motif such as a nitrobenzyl photocleavable linker which may be inserted into selected locations of the prepolymer. In some embodiments, a nitrobenzyl photocleavable linker may include a 1-methinyl, 2-nitrobenzyl moiety configured to be photocleavable. In other embodiments, the photocleavable linker may include a benzoin moiety, a 1, 3 nitrophenolyl moiety, a coumarin-4-ylmethyl moiety or a 1-hydroxy 2-cinnamoyl moiety. A cleavage motif may be utilized to remove the solidified polymer network of an isolation structure. In other embodiments, the polymer may include cell recognition motifs including but not limited to a RGD peptide motif, which is recognized by integrins. Additional cleavable moieties useful for in situ-generated structures are described below.

Polyethylene glycol moieties linked to acrylamide reactive moiety. One type of polymer, amongst the many polymers that may be used, is polyethylene glycol diacrylate (PEGDA) or polyethylene glycol acrylamide (diacrylamide, multi-armed acrylamide or substituted versions as described herein).

Polyethylene glycol molecules linked to a norbornene reactive moiety. In other embodiments, a polyethylene glycol polymer molecule including a polyethylene glycol moiety covalently linked to a norbornene reactive moiety may be used as part of a prepolymer, e.g., flowable polymer, composition to be solidified in-situ within a microfluidic device. A first polyethylene glycol polymer molecule will be linked to a crosslinker, and the crosslinker may further be linked to a second polyethylene glycol molecule via a norbornene reactive moiety linked to a second polyethylene glycol moiety of the second polyethylene glycol molecule. The norbornene containing polyethylene glycol molecules react with crosslinkers including thiols, upon photoinitiation in the presence of an initiator to produce thiol-ene crosslinked hydrogels in-situ.

The composition may include: a first and a second polyethylene glycol polymer molecule, each comprising a respective polyethylene glycol moiety and a covalently linked reactive moiety Rx; a crosslinker molecule comprising a first reactive moiety Rxp disposed at a first end of a linker L moiety and a second reactive moiety Rxp disposed at a second end of the linker L moiety, wherein each of the first and the second crosslinker moiety Rxp is configured to be activatable to react with the respective reactive moiety Rx of the first and the second polyethylene polymer molecules, wherein the first and the second polyethylene polymer molecule comprises different polyethylene glycol moieties or each comprises a same polyethylene glycol moiety.

The first reactive moiety Rxp and the second reactive moiety Rxp of the first and the second polyethylene glycol molecule may be a norbornenyl moiety. In some embodiments, the norbornenyl moiety may be a substituted norbornenyl moiety. In some embodiments, the substituted norbornenyl moiety may have a hydrophilic substituent, such as but not limited to hydroxy or carboxylate. The substituent does not inhibit the reactivity of the norbornenyl moiety. The norbornenyl moiety is configured to react with a thiol moiety of a crosslinker molecule to form the hydrogel.

The first polyethylene glycol moiety and/or the second polyethylene glycol moiety may include a 1-arm, 2-arm, 4-arm or 8-arm polyethylene glycol moiety. In some embodiments, each arm of the first polyethylene glycol moiety and/or the second polyethylene glycol moiety may include the covalently linked reactive moiety Rx. In other embodiments, when the first polyethylene glycol moiety and/or the second polyethylene glycol moiety has a 2-arm, 4-arm, or 8-arm polyethylene moiety structure, at least one arm of the first polyethylene glycol moiety and/or the second polyethylene glycol moiety may include the covalently linked reactive moiety Rx and at least one arm of the first polyethylene glycol moiety does not have a covalently linked reactive moiety Rx. For example, an 8-arm polyethylene glycol moiety may have seven arms including a reactive moiety Rx and one arm that does not include the reactive moiety Rx. In some embodiments, an 8-arm polyethylene glycol moiety may have 6-arms, 5-arms, 4-arms, 3-arms, or 2-arms having the reactive moiety Rx while the other arms do not have the reactive moiety Rx.

The first polyethylene glycol moiety may have a molecular weight from about 500 Da to about 40KDa; from about 1 kDa to about 25KDa; from about 5KDa to about 25KDa; from about 5KDa to about 20KDa; about 5KDa to about 15KDa; or about 5KDa to about 10KDa. The second polyethylene glycol moiety may have a molecular weight from about 500 Da to about 40KDa; from about 1KDa to about 25KDa; from about 5KDa to about 25KDa; from about 5KDa to about 20KDa; about 5KDa to about 15KDa; or about 5KDa to about 10KDa.

In some embodiments, the first polyethylene glycol moiety may have a molecular weight of about 10KDa and the second polyethylene glycol moiety may have a molecular weight of about 10KDa. In other embodiments, the first polyethylene glycol moiety may have a molecular weight of about 10KDa and the second polyethylene glycol moiety may have a molecular weight of about 20KDa. When the first polyethylene glycol moiety has a different molecular weight from that of the second polyethylene glycol moiety, the hydrogel once formed will include a mixture of molecules, e.g., some hydrogel molecules will have a 10KDa polyethylene glycol moiety linked through the crosslinker to a second 10KDa polyethylene glycol moiety; some hydrogel molecules will have a 10KDa polyethylene glycol moiety linked through the crosslinker to a 20KDa polyethylene glycol moiety; and some hydrogel molecules will have a 20KDa polyethylene glycol moiety linked through the crosslinker to a second 20KDa polyethylene glycol moiety.

In some embodiments, the first polyethylene glycol molecule and the second polyethylene molecule may be present in the composition in a ratio from about 1:100 to about 100:1. In some further embodiments, the ratio of the first polyethylene glycol molecule and the second polyethylene molecule may be from about 1:1; about 1:2; about 1:3; about 1:4; about 1:5; about 1:6; about 1:7; about 1:8; about 1:9; about 1:10; about 10:1; about 9:1; about 8:1; about 7:1; about 6:1; about 5:1; about 4:1; about 3:1; or about 2:1.

Crosslinker molecule. For the compositions described herein, the crosslinker molecule comprises a first thiol at a first end of the crosslinker molecule and a second thiol moiety at a second end of the molecule, where the first thiol moiety and the second thiol moiety are each configured to reach with a norbornenyl group of either the first or the second polyethylene glycol molecule including a reactive moiety, where the reactive moiety may be a norbornenyl moiety.

In some embodiments, the crosslinker molecule may include a vicinal-diol moiety. A vicinal diol (vic-diol) moiety of the crosslinker provides an in-situ generated hydrogel that may be reversed, e.g, removed or dissolved at a later timepoint, by contact with a periodate (Sodium Periodate). This class of hydrogels are typically referred to herein as a reversible hydrogel.

Vic-diol containing crosslinker. In some embodiments of a crosslinker having a vic-diol moiety, the crosslinker has a structure having a molecular formula of Formula II:

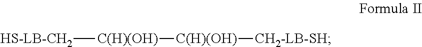

where each instance of linker backbone LB is independently selected to comprise 0 to 200 non-hydrogen atoms selected from any combination of silicon, carbon, nitrogen, oxygen, sulfur and phosphorus atoms. In some embodiments, the linker backbone LB may have a linear backbone having carbon atoms. In some embodiments, the linear backbone having carbon atoms does not include any silicon, nitrogen, oxygen, sulfur or phosphorus atoms. When referring to the linear backbone LB having carbon atoms, this does not exclude substitution of hydrogen atom substituents of the carbon backbone with other types of moieties containing alcohols, sulfonates, carboxylic acids and the like. In some embodiments, the crosslinker molecule is dithiothreitol.

Nonreversible crosslinker. In other embodiments, the crosslinker molecule has a structure of Formula V:

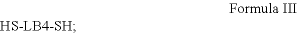

where linker backbone LB4 comprises 3 to 200 non-hydrogen atoms selected from any combination of silicon, carbon, nitrogen, oxygen, sulfur and phosphorus atoms. In some embodiments, the linker backbone LB4 may have a linear backbone having carbon atoms. In some embodiments, the linear backbone having carbon atoms does not include any silicon, nitrogen, oxygen, sulfur or phosphorus atoms. When referring to the linear backbone LB4 having carbon atoms, this does not exclude substitution of hydrogen atom substituents of the carbon backbone with other types of moieties containing alcohols, sulfonates, carboxylic acids and the like. A crosslinker of Formula V provides a hydrogel that is non-reversible, e.g, not dissolvable. In some embodiments, the crosslinker is Sodium 2,3-dimercaptopropanesulfonate monohydrate.

Inhibitor. In some embodiments, the compositions may further include an inhibitor configured to inhibit reaction of the crosslinker with the reactive moiety of the first and/or the second polyethylene glycol polymer molecule and/or of the reactive moiety of the first and/or the second polyethylene glycol polymer molecules upon standing in solution. The inhibitor may be sodium ascorbate, MEHQ, or 4-hydroxy TEMPO. The composition including the first and the second polyethylene glycol polymer molecule, and the crosslinker molecule may further include an inhibitor. In some embodiments, the inhibitor is present within the composition at a concentration from about 0.5 millimolar to about 1.5 millimolar. In other embodiments, the composition may include another component having a photoinitiator and a second amount of the inhibitor. The inhibitor in the component having the photoinitiator may have the inhibitor present in a range from about 5 millimolar to about 20 millimolar. The composition component containing the photoinitiator and second amount of inhibitor may be separately packaged and not combined with the compositions including the first and the second polyethylene glycol polymer molecules until the compositions are introduced into the microfluidic device.

Thiol-Ene Hydrogels. The compositions described above provide a thiol-ene hydrogel once formed, and include a first polyethylene glycol polymer moiety covalently linked to a first end of a crosslinker moiety, wherein a second end of the crosslinker moiety is covalently linked to a second polyethylene glycol polymer moiety, wherein the first and the second polyethylene polymer molecule comprises different polyethylene glycol moieties or may each comprise a same polyethylene glycol moiety.

The hydrogel has a structure of Formula I:

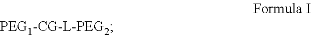

where PEG1 is the first polyethylene glycol moiety and PEG2 is the second polyethylene glycol moiety; CG is a coupled group formed from the reaction of the Rx moiety and the RxP moiety; and L is the crosslinker moiety, comprising a linear portion wherein a backbone of the linear portion comprises 1 to 200 non-hydrogen atoms selected from any combination of silicon, carbon, nitrogen, oxygen, sulfur and phosphorus atoms. The CG coupled group is a norbornenyl moiety coupled to a thiol moiety, e.g., a thiol-ene coupled group.

The first polyethylene glycol moiety and/or the second polyethylene glycol moiety may include a 1-arm, 2-arm, 4-arm or 8-arm polyethylene glycol moiety. In some embodiments, each arm of the first polyethylene glycol moiety and/or the second polyethylene glycol moiety may include the covalently linked reactive moiety Rx. In other embodiments, when the first polyethylene glycol moiety and/or the second polyethylene glycol moiety has a 2-arm, 4-arm, or 8-arm polyethylene moiety structure, at least one arm of the first polyethylene glycol moiety and/or the second polyethylene glycol moiety is covalently linked to the first end of the crosslinker moiety and at least one arm of the first polyethylene glycol moiety and/or the second polyethylene glycol moiety is not covalently linked to a second crosslinker moiety. For example, an 8-arm polyethylene glycol moiety may have seven arms that have been crosslinked to respective crosslinker moieties and one arm that was not crosslinked. In some embodiments, an 8-arm polyethylene glycol moiety may have 6-arms, 5-arms, 4-arms, 3-arms, or 2-arms that are crosslinked to respective crosslinker moieties while the other arms are not crosslinked.

The first polyethylene glycol moiety may have a molecular weight from about 500 Da to about 40KDa; from about 1 kDa to about 25KDa; from about 5KDa to about 25KDa; from about 5KDa to about 20KDa; about 5KDa to about 15KDa; or about 5KDa to about 10KDa. The second polyethylene glycol moiety may have a molecular weight from about 500 Da to about 40KDa; from about 1KDa to about 25KDa; from about 5KDa to about 25KDa; from about 5KDa to about 20KDa; about 5KDa to about 15KDa; or about 5KDa to about 10KDa.

In some embodiments, the first polyethylene glycol moiety may have a molecular weight of about 10K Da and the second polyethylene glycol moiety may have a molecular weight of about 10K Da. In other embodiments, the first polyethylene glycol moiety may have a molecular weight of about 10K Da and the second polyethylene glycol moiety may have a molecular weight of about 20K Da. When the first polyethylene glycol moiety has a different molecular weight from that of the second polyethylene glycol moiety, the hydrogel once formed will include a mixture of molecules, e.g., some hydrogel molecules will have a 10KDa polyethylene glycol moiety linked through the crosslinker to a second 10KDa polyethylene glycol moiety; some hydrogel molecules will have a 10KDa polyethylene glycol moiety linked through the crosslinker to a 20KDa polyethylene glycol moiety; and some hydrogel molecules will have a 20KDa polyethylene glycol moiety linked through the crosslinker to a second 20KDa polyethylene glycol moiety.

In some embodiments, the first polyethylene glycol molecule and the second polyethylene molecule may be present in the composition in a ratio from about 1:100 to about 100:1. In some further embodiments, the ratio of the first polyethylene glycol molecule and the second polyethylene molecule may be from about 1:1; about 1:2; about 1:3; about 1:4; about 1:5; about 1:6; about 1:7; about 1:8; about 1:9; about 1:10; about 10:1; about 9:1; about 8:1; about 7:1; about 6:1; about 5:1; about 4:1; about 3:1; or about 2:1.

The crosslinker moiety L is linked to PEG1 through a first thiol-ene coupled group at a first end of the crosslinker molecule and linked to PEG1 through a second thiol-ene coupled group at a second end of the moiety.

In some embodiments, crosslinker moiety L comprises a vic-diol. A hydrogel having a vicinal diol (vic-diol) moiety within may be reversed, e.g, removed or dissolved at a later timepoint, by contact with a periodate (Sodium Periodate). This class of hydrogels are typically referred to herein as a reversible hydrogel.

In some embodiments, the crosslinker L moiety has a molecular formula:

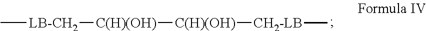

wherein each instance of linker backbone LB is independently selected to comprise 0 to 200 non-hydrogen atoms selected from any combination of silicon, carbon, nitrogen, oxygen, sulfur and phosphorus atoms. In some embodiments, the linker backbone LB may have a linear backbone having carbon atoms. In some embodiments, the linear backbone having carbon atoms does not include any silicon, nitrogen, oxygen, sulfur or phosphorus atoms. When referring to the linear backbone LB having carbon atoms, this does not exclude substitution of hydrogen atom substituents of the carbon backbone with other types of moieties containing alcohols, sulfonates, carboxylic acids and the like. In some embodiments, the crosslinker molecule is derived from a threitol moiety.

In yet other embodiments, the crosslinker moiety of the hydrogel has a structure of Formula VIII:

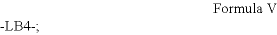

wherein linker backbone LB4 comprises 3 to 200 non-hydrogen atoms selected from any combination of silicon, carbon, nitrogen, oxygen, sulfur and phosphorus atoms. A hydrogel having a crosslinker moeity of Formula VIII, is a non-reverisble hydrogel. In some embodiments, the linker backbone LB2 may have a linear backbone having carbon atoms. In some embodiments, the linear backbone having carbon atoms does not include any silicon, nitrogen, oxygen, sulfur or phosphorus atoms. When referring to the linear backbone LB2 having carbon atoms, this does not exclude substitution of hydrogen atom substituents of the carbon backbone with other types of moieties containing alcohols, sulfonates, carboxylic acids and the like. In some embodiments, the crosslinker moiety is derived from Sodium 2,3-dimercaptopropanesulfonate monohydrate.

One type of polymer, amongst the many polymers that may be used, is polyethylene glycol diacrylate (PEGDA) or polyethylene glycol acrylamide (diacrylamide, multi-armed acrylamide or substituted versions as described herein).

Photoactivated polymerization may be accomplished using a free radical initiator Igracure® 2959 (BASF), a highly efficient, non-yellowing radical, alpha hydroxy ketone photoinitiator, is typically used for initiation at wavelengths in the UV region (e.g., 365 nm), but other initiators may be used. An example of another useful photoinitiator class for polymerization reactions is the group of lithium acyl phosphinate salts, of which lithium phenyl 2, 4, 6,-trimethylbenzolylphosphinate has particular utility due to its more efficient absorption at longer wavelengths (e.g., 405 nm) than that of the alpha hydroxy ketone class. Another initiator that may be used are water soluble azo initiators, such as 2, 2-Azobis [2-methyl-N-(2-hydroxyethyl) propionamide]. The initiator may be present within the flowable polymer solution at a concentration of about 5 millimolar, about 8 millimolar, about 10 millimolar, about 12 millimolar, about 15 millimolar, about 18 millimolar, about 20 millimolar, about 22 millimolar, about 25 millimolar, about 28 millimolar, about 30 millimolar, about 35 millimolar, or about 40 millimolar.

Crosslinking may be performed by photopatterning of linear or branched PEG polymers, free radical polymerization of PEG acrylates or PEG acrylamides, and specifically tailored chemical reactions such as Michael addition, condensation, Click chemistry, native chemical ligation and/or enzymatic reactions. In particular, photopatterning of crosslinking may be used to gain precise control of extent of the physical extent of the hydrogel barrier as well as the degree of crosslinking, as described in the following section and in the Examples.

Inhibitors may be included within the flowable polymer solution to ensure precise control of photopatterning and to prevent extraneous or undesired polymerization. One useful inhibitor is hydroquinone monomethyl ether, MEHQ, but other suitable inhibitors may be used. The inhibitor may be present in the flowable polymer solution at a concentration of about 1 millimolar, about 2 millimolar, about 5 millimolar, about 10 millimolar, about 15 millimolar, about 20 millimolar, about 25 millimolar, about 30 millimolar, about 35 millimolar, about 40 millimolar or more, as needed to provide the photopatterning control desired.

Tuneable permeability. One aspect of performing assays using a hydrogel in a situ-generated structure is to determine what species is desired to gain access to an area of interest. Selection of the chemical nature of the polymer (for example, molecular weight range, number of cross linkable moieties per polymer unit (linear, 2 arm, 4 arm, 8 arm, star or comb polymer), mixtures of polymers), the amount of initiator, and mode of polymerization are variables that may be modified to tune the hydrogel barrier formed. Generally, the initiator is a photoinitiator. Photopatterning provides precise control of the geometry of the polymerization as well as the extent of polymerization, and changes in exposure time and power of the illumination also can provide more control to arrive at a desired type of porosity and degree of robustness of the polymerized feature.

In many variations, polymer selection may depend upon the biocompatibility of the polymer species, and may be related to the specific application to which a hydrogel in situ-generated barrier may be used.

In some variations, the hydrogel may be a polyethylene glycol polymer or a modified polyethylene glycol polymer.

A wide range of molecular weights of the flowable polymer may be suitable, depending upon the structure of the polymer. In some embodiments, the flowable polymer may have a molecular weight of about 500 Da to about 20kDA, or about 500 Da, about 1 kDa, about 3 kDa, about 5 kDa, about 10 kDa, about 12 kDa, about 15 kDa, about 20 kDa or any value therebetween. A useful star type polymer may have Mw (weight average molecular weight) in a range from about 500 Da to about 20 kDa (e.g., four arm polymer), or up to about 5 kDa for each arm, or any value therebetween. In some embodiments, a polymer having a higher molecular weight range, may be used at lower concentrations in the flowable polymer, and still provide an in situ-generated structure that may be used in the methods described herein.

Reversing/removing/minimizing the in situ-generated isolation structure. A number of mechanisms may be used to remove or reduce the in situ-generated hydrogel barrier when there is no further purpose for it. For example, once an assay is completed and desirable biological cells have been identified, it may be useful to remove the hydrogel barrier in order to export the cells and/or continue culturing and expanding the biological cell demonstrating desirable activities or properties.

In other variations, such as is described herein, laser initiated bubbles may provide forces that can deform or disrupt the hydrogel barrier, permitting export of the cells.

Hydrolytic susceptibility: Porogens, including polymers which are incapable of being chemically linked to the photoinitiated polymer(s), may be included when forming the hydrogel barrier. The degree/size of openings within the formed hydrogel can customize the hydrolysis rate via accessibility within the hydrogel barrier). In other embodiments, the pores formed may be employed to permit secreted materials or chemical reagents to pass through the hydrogel barrier but prevent a cell from moving into, out of, and/or through the isolation structure. In other embodiments, degradability of these polymers may be increased by introducing degradable segments such as polyester, acetal, fumarate, poly(propylene fumarate) or polyhydroxyacids into polymers (e.g., PEG polymers).

Reducing agents: PEG may be formed with disulfide linkages at intervals along the macromere, which may be random or predetermined. The disulfide bonds may be broken by Dithiothreitol (DTT), mercaptoethanol, or TCEP.

Thermal: poly N-isopropylacrylamide (PNIPAm) or other suitable LCST polymers may be used to introduce hydrogel barriers upon heating. They may be removed by decreasing the temperature of the formed polymer hydrogel barrier. The polymers may include ELPs or other motifs that also permit removal by other mechanisms such as hydrolysis or proteolysis. In particular, PNIPAm may be used to create a surface for adherent cells, but then switched to permit export.

Proteolytic susceptibility: Hydrogels may have any sort of peptide sequence engineered in, such that selective proteolysis upon a selected motif by a selected protease can remove/reverse/or minimize a hydrogel isolation structure. Some classes of modified PEG include PEG having elastin like peptide (ELP) motifs and/or having peptide motifs for susceptibility to a variety of proteases (enzyme sensitive peptide ESP). A large number of these motifs are known. One useful motif is RGD which may be constrained to be cyclic.

Osmotic susceptibility: Calcium concentration/other osmotic strategies can be employed to degrade and remove a hydrogel barrier. As above, changes of media flowed through the channel or flow region may dimensionally swell or de-swell hydrogel barriers.

Photocleavage: As described above, if a polymer of the solidified polymer network includes a photocleavable moiety, directing illumination of an exciting wavelength to the solidified polymer network will cause cleavage within sections of the solidified polymer network. This cleavage may provide complete or partial disruption of the solidified polymer network, thereby removing or reducing the hydrogel barrier.

In some applications, the hydrogel barrier may not be removed but may simply be swelled or de-swelled using light or medialsolvent changes. Some types of hydrogels may incorporate moieties that respond reversibly to light (for example, change regiochemistry about a rigid bond; form reversible crosslinks within the polymer, or form/break ion pairs).

### Microfluidic Device/System Feature Cross-Applicability

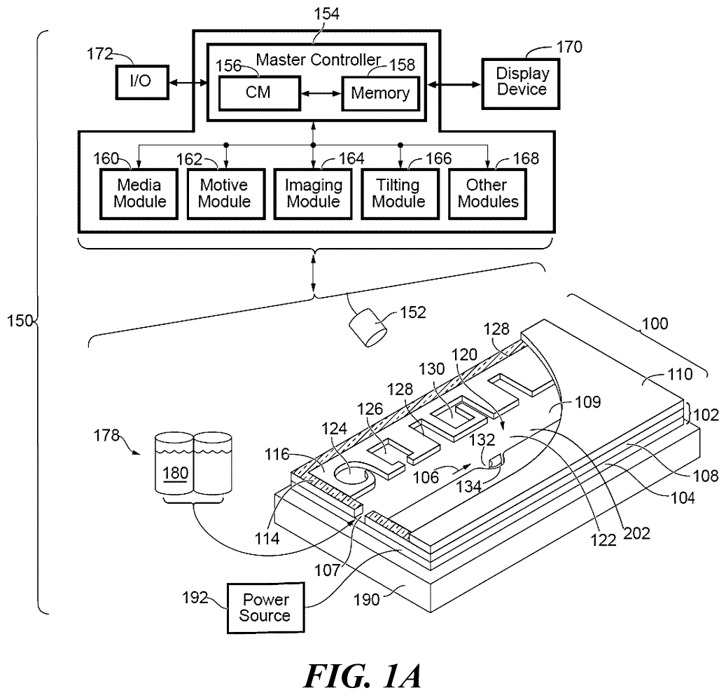

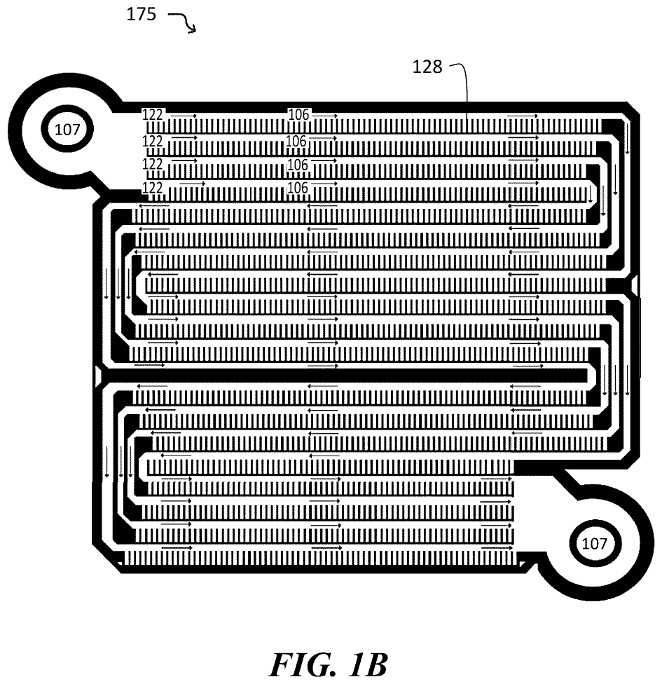

It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials-and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n-1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

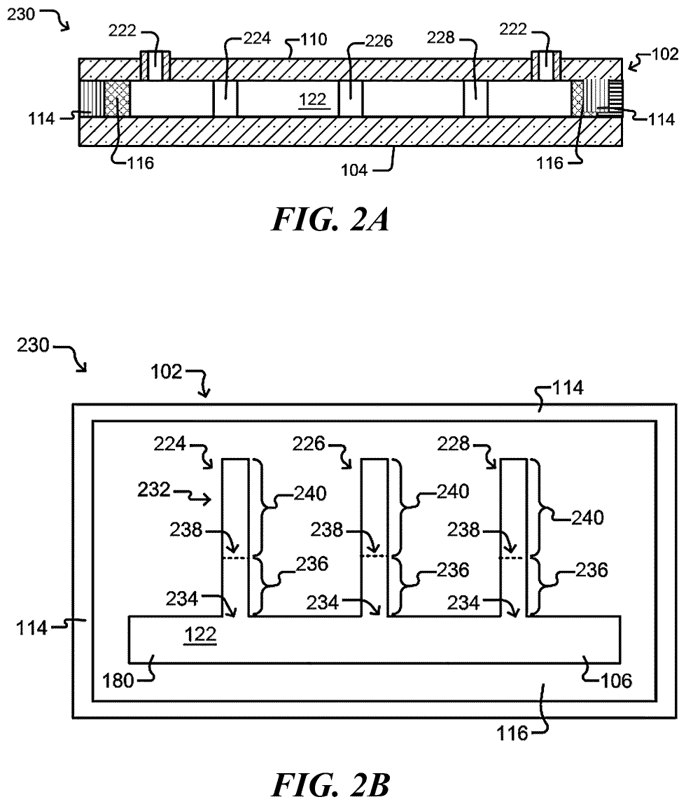

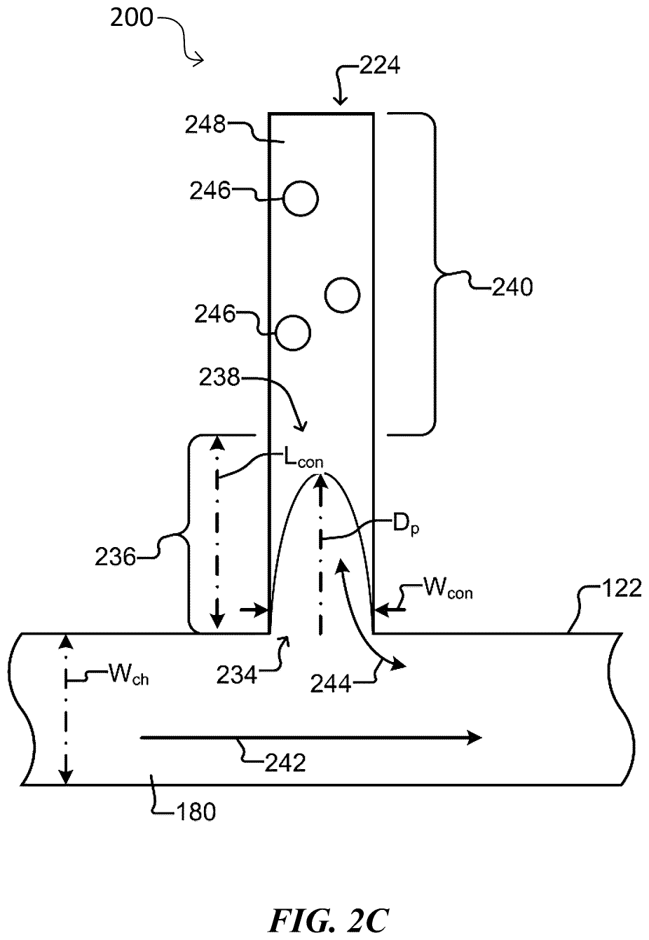

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%, 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width Wen of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

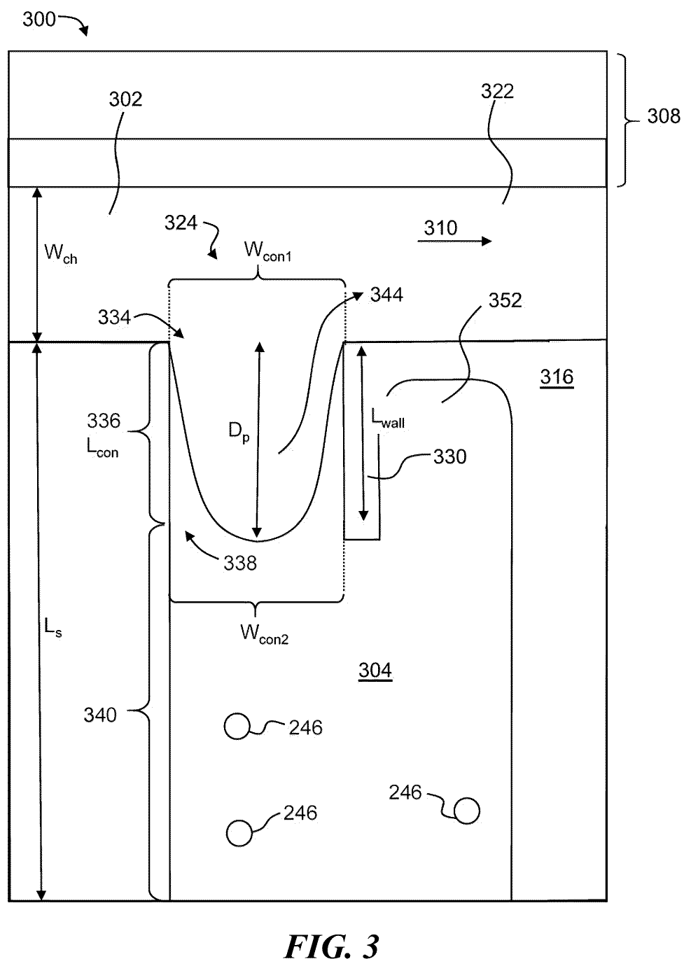

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Lₛ, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n-1 openings can be valved. When the n-1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length Leon that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size Wc (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size Wo (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion D₀ of the molecule. For example, the Do for an IgG antibody in aqueous solution at about 20° C. is about 4.4×10−7 cm²/sec, while the kinematic viscosity of cell culture medium is about 9×10−4 m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W^{con1} of a proximal opening (e.g., 234 or 334) of a connection region of a sequestration pen may be the same as a width W_{con2} of the distal opening (e.g., 238 or 338) to the isolation region thereof. In some embodiments, the width W^{con1} of the proximal opening may be different than a width W_{con2} of the distal opening, and W^{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10⁻⁵⁰⁰ microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono-or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG Mw<100,000 Da) or alternatively polyethylene oxide (PEO, Mw>100,000). In some embodiments, a PEG may have an Mw of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules: second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about Inm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula A, as shown below. Alternatively, the covalently linked coating material may be formed in a two-part sequence, having a structure of Formula B, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

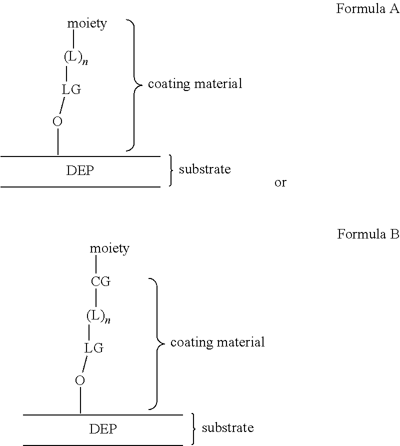

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rx and a reactive pairing moiety Rpx (i.e., a moiety configured to react with the reactive moiety Rx). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology (ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Patent No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Patent Application No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

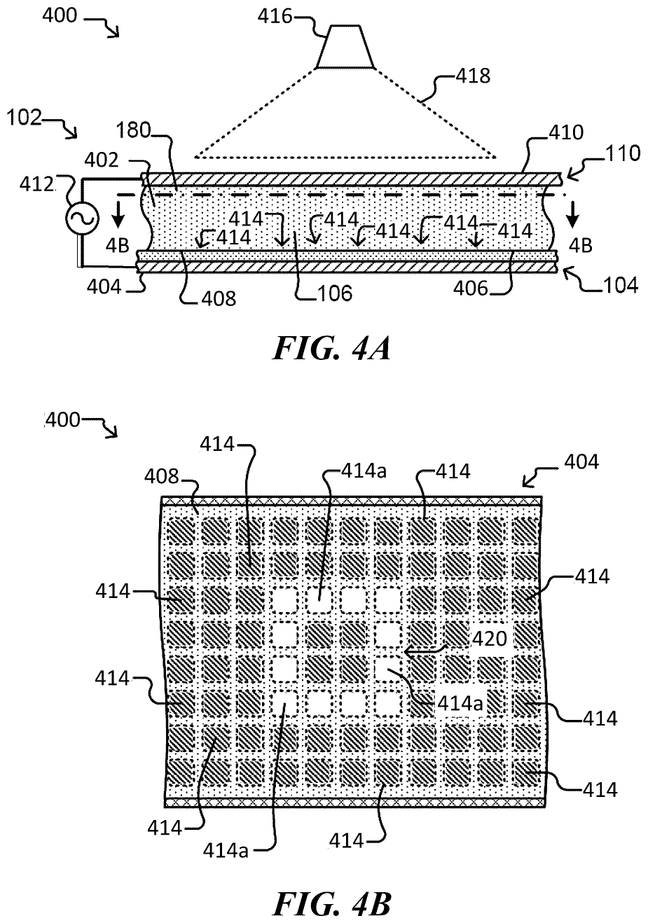

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 400 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 414 thus need not be fixed, but can correspond to the light pattern 418. Examples of microfluidic devices having a DEP configuration comprising a photoconductive layer such as discussed above have been described, for example, in U.S. Patent No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x-and y-axes), a vertical orientation (i.e., at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

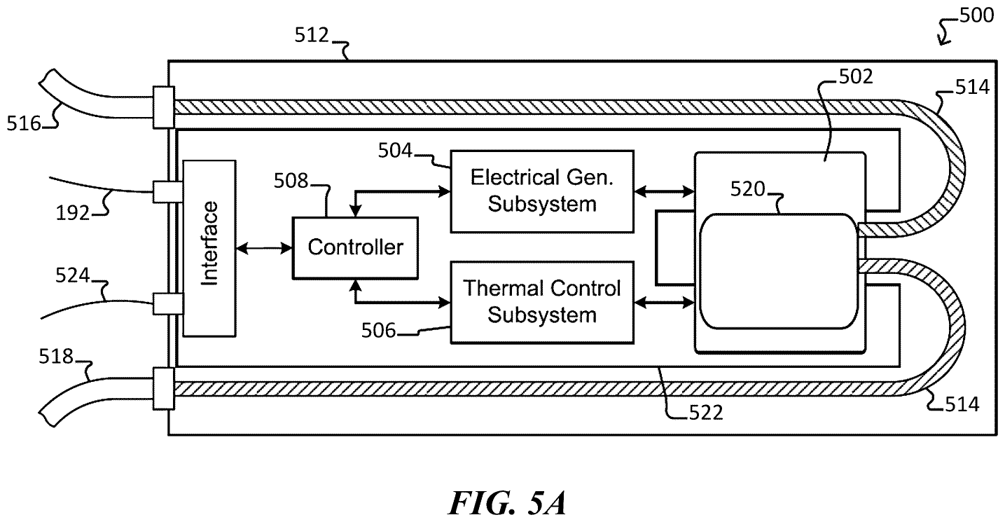

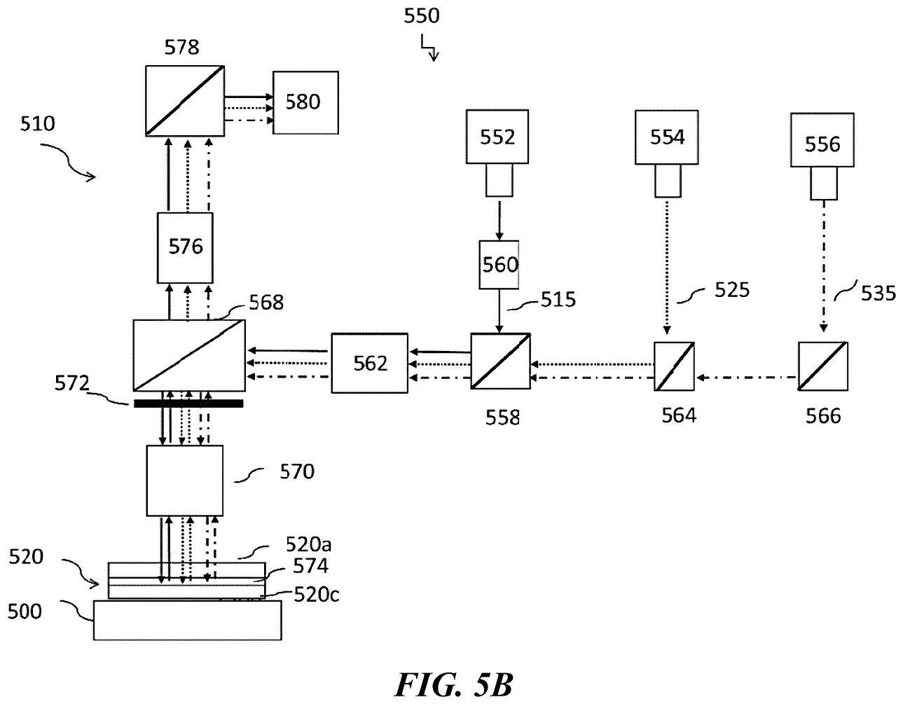

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 522, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 5A the controller 508 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns x10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 564 and be transmitted therefrom to the first beam splitter 558, and onward to the first tube lens 562. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 510. The objective lens 570 can have one or more magnification levels available such as, 4X, 10X, 20X.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 510 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), 10,010,882 (White et al.), and 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

### EXAMPLES

System and Microfluidic device: An OPTOSELECT™ device, a nanofluidic device controlled by an optical instrument, BEACONR were employed (Both are manufactured by Berkeley Lights, Inc.) The instrument includes: a mounting stage for the chip coupled to a temperature controller; a pump and fluid medium conditioning component; and an optical train including a camera and a structured light source suitable for activating phototransistors within the chip. The OPTOSELECT™ device includes a substrate configured with OptoElectroPositioning (OEP™) technology, which provides a phototransistor-activated OET force. The chip also included a plurality of microfluidic channels, each having a plurality of NANOPEN™ chambers (or chambers) fluidically connected thereto. The volume of each chamber is around 1×10⁶ cubic microns.

Priming regime: 250 microliters of 100% carbon dioxide was flowed in to the OPTOSELECT™ device at a rate of 12 microliters/sec, followed by 250 microliters of PBS containing 0.1% PLURONICK F27 (LIFE TECHNOLOGIESR Cat #P6866) flowed in at 12 microliters/sec, and finally 250 microliters of PBS flowed in at 12 microliters/sec. Introduction of a Wetting Solution follows, which introduces a conditioned surface to the surfaces within the microfluidic device. The details of the surface and its introduction are described in US Application Publication US 2016/0312165, filed on Apr. 22, 2016 and U.S. Application Publication No. US2019/0275516, filed on Nov. 20, 2018, each of which disclosures are herein incorporated by reference in its entirety.

System Preparation. OPTOSELECT™ chips were loaded onto Beacon instrument for a sequence of pre-workflow operations. The Wetting Solution was introduced to the chip and then incubated to functionalize the surfaces. DI water was then flushed through each chip to remove the Wetting Solution. After wetting and priming, the Beacon instrument automatically located the fiducial markers on the chips for x-y stage and focus calibration. Reference imaging was then performed.

Single-Cell Loading. Cells were loaded in a manner as previously described in U.S. Pat. No. 11,170,200 (Kim et al.), filed on May 31, 2019 and granted Nov. 9, 2021, and U.S. Publication No. US2021/0209752 (Tenney et al.), filed on Nov. 24, 2020. Single cells were identified automatically by the Beacon instrument control software using a convolutional neural network algorithm. A positioning strategy was then automatically implemented to maximize loading throughput using the OEP technology. Residual cells in the channels were flushed to waste after loading.

### Example 1: Evaluating Production of a Virus-Producing Cell Using Adopted Recombinase Polymerase Amplification Technology

This experiment was conducted to evaluate the productivity of a producer cell cultured in a sequestration pen of a microfluidic device using recombinase polymerase amplification (RPA, TwistAmp® Liquid exo) assay. The cells tested in this experiment were PCL2 cells, which were made from Hela cells transformed with AAV rep gene and cap gene. The cells were also transfected with a AAV vector having a GFP coding gene (i.e., the payload of this experiment) flanked with ITRs. The PCL2 cells can be induced to replicate AAV vector, packaging AAV particles, and release the AAV particles by infecting the cells with adenoviruses. Among the PCL2 cells used in this experiment, some of the cells can produce a higher percentage of AAV particles encapsulating the AAV vector comprising a GFP coding gene while some might produce a higher percentage of empty AAV particles.

Loading cells and splitting clone. A microfluidic device as described herein having a plurality of sequestration pens was used for this experiment. A fluidic medium comprising a plurality of the PCL2 cells was introduced in the microfluidic device. Single cells were disposed into each selected sequestration pen of the plurality of the sequestration pens, and each selected sequestration pen was spaced with an empty sequestration pen, that is, a sequestration pen without the cells disposed therein. The disposed cells were cultured on chip and expanded into a clonal population respectively.

In this experiment, each sequestration pen with cell disposed therein was designated as an assay chamber, the empty sequestration pen next to the assay chamber was designated as a preserving chamber, and the two were viewed together as a culture unit. A subset of each clonal population in a sequestration pen was moved to the preserving chamber by DEP force. The remaining cells of that clonal population were kept in the assay chamber of the culture unit. Then, the preserving chamber was sealed with an in situ-generated cap to preserve the cells therewithin from being affected by the assay to be performed in the assay chamber.

RPA. Before the RPA assay was performed, the microfluidic device was washed by perfusing PBS into the flow region. Then, the reagents required for the RPA assay were loaded separately. In the 1st load, a mixture comprising recombinase, polymerase, primers, and probes was introduced in PBS (these reagents were E-mix, Core-mix, primers, and exo probe of the commercial kit TwistAmp® Liquid exo). In the 2nd load, a mixture comprising recombinase, polymerase, primers, probes, and dNTPs was introduced with a crowding reagent (PEG) (these reagents were E-mix, Core-mix, primers, exo probe, dNTPs, and buffer of the commercial kit TwistAmp® Liquid exo). After the reagents properly diffused in the assay chambers, a 3rd load comprising recombinase, polymerase, primers, probes, dNTPs, the crowding reagent, and MgOAc was introduced. The MgOAc is an initiator of the commercial product used to activate the RPA reaction. Then, 40 ul of filtered air was introduced to replace the liquid in the microfluidic channel and seal the sequestration pens. Images were taken to detect a signal associated with the exo probe. The sequences of the primers and probes used in this experiment were shown in the following table.

Table: RPA primers and probes

Forward primer (SEQ ID NO.: 01) CATCTTCTTCAAGGACGACGGCAACTAC
Reverse primer (SEQ ID NO.: 02) GTTCTTCTGCTTGTCGGCGGTGATATAG
Exo probe (SEQ ID NO.: 03) TCGAGGGCGACACCCTGGTGAACCGCATCGGCTG

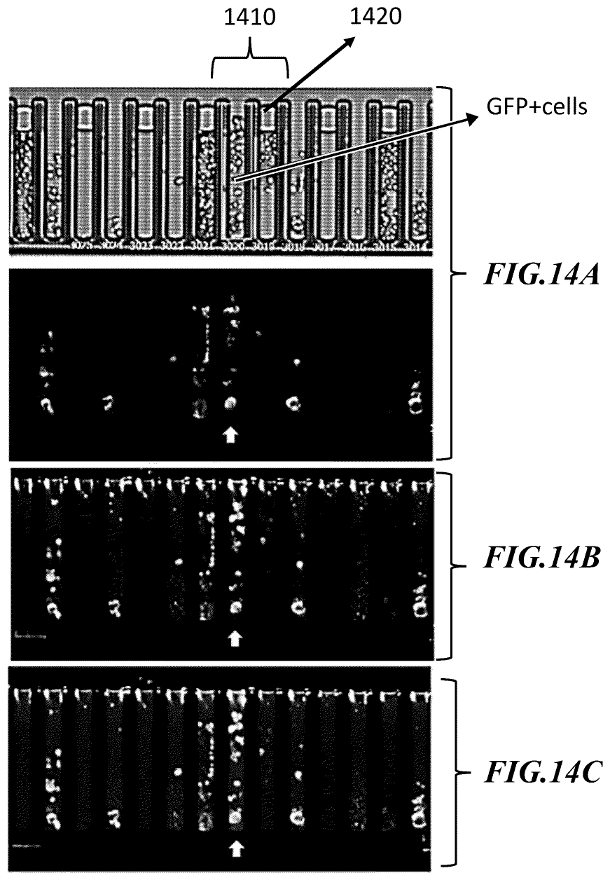

Results. Cells that were transfected and replicating the GFP coding genes successfully can be identified by detecting the GFP signal. Among all sequestration pens, there were four sequestration pens identified to be GFP+ cells. FIGS. 14A-14C shows images of detecting the RPA probes, and one of the four sequestration pens of GFP+ cells was marked in this field of view.

FIG. 14A shows images taken before the RPA assay is performed. The top image is a brightfield image shows several culture units (for example, culture unit 1410). Each culture unit had two sequestration pens comprising cells. One of the two sequestration pens sealed with in situ-generated cap 1420 was the preserving chamber, and the other one was the assay chamber for the RPA assay to be performed. The bottom image is a fluorescent image detecting the RPA probe. Note that the fluorescent signals detected in FIG. 14A (bottom) was associated with the probe instead of GFP. The image shows that there was background noise before the RPA reaction was performed.

FIG. 14B shows a fluorescent image taken after the 1st load and the 2nd load were completed but before the 3rd loading was performed. FIG. 14C is a fluorescent image taken 10 minutes after the 3rd load was made. Note that the detected signals in the bottom image of FIG. 14A did not increase in FIG. 14B and FIG. 14C. This proved those signals were background noise and irrelevant to the existence of the payload of interest. In comparison, an increase of signal can be observed in the sequestration pen containing the GFP+ cells (see the arrowed sequestration pen). The increase represents the existence of the payload of interest. Note also that the preserving chamber of the culture unit 1410 did not exhibit notable signal, showing that the in situ-generated cap blocked the RFA reagents from entering the preserving chamber.

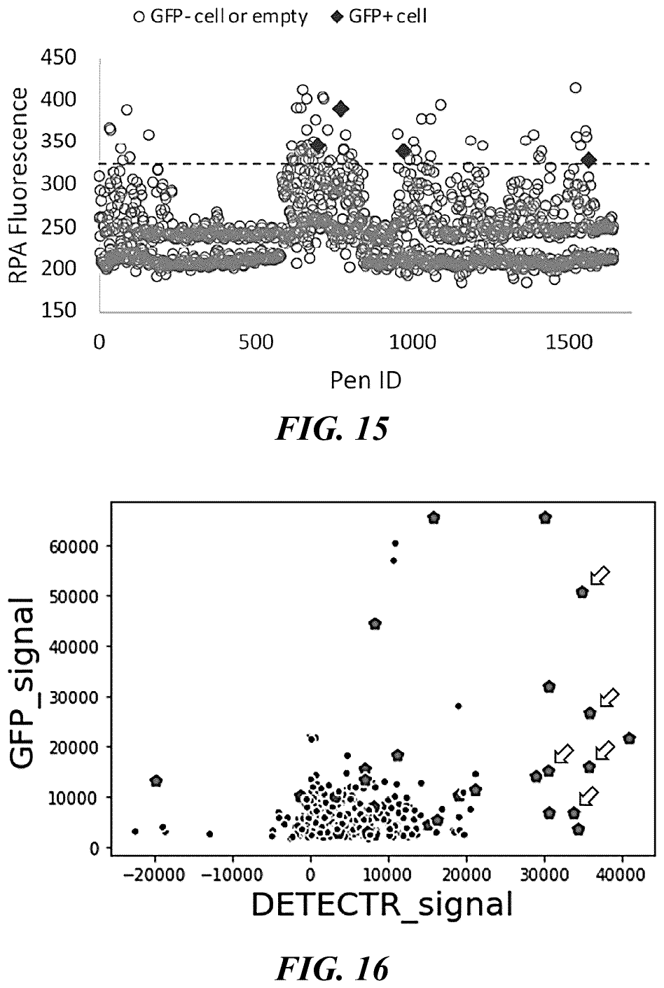

FIG. 15 shows that over a thousand chambers tested, the four sequestration pens comprising GFP+ cells (diamond signs in the figure) were among the top 62 sequestration pens exhibiting signals stronger than a selected threshold (the dotted line in FIG. 15). That said, the recombinase polymerase amplification is able to be performed on the microfluidic device to down select candidate cells having the target nucleotide acid sequence. In other words, based on the results of the RPA assay, only the cells exhibiting RPA signals are needed to be exported for further verification as the cells carrying and replicating the payload were enriched in the group of RPA positive cells. In other words, based on the results of the RPA assay, there is no need to verify all cells cultured in the microfluidic device.

### Example 2: Evaluating Production of a Virus-Producing Cell Using CRISPR Technology

This experiment was conducted using CRISPR-based DETECTR assay to detect a payload in a producer cell cultured in a sequestration pen of a microfluidic device. The cells tested and the payload were as described in Example 1. Cells were loaded and cultured, and clonal populations therefrom were split as described in Example 1.

DETECTR. Before the DETECTR assay was performed, a trypsin solution was introduced into the microfluidic device. The trypsin solution diffused into the flow region and sequestration pens that were not capped by the in situ-generated cap. Without being bound by theory, the trypsin was used to eliminate the endogenous nucleases in the assay chambers. In some embodiments, other types of proteinase or other approaches can be used for this purpose. Then, the DETECTR probes (a TA-rich probe, SEQ ID NO.: 04) were introduced into the microfluidic device in buffer (HEPES BPS). The sequestration pens were sealed by fluorinated oil. Images were taken to detect fluorescent signals (pre-assay signals) associated with the probe.

The oil cap was then removed. A mixture of Cas-12a: crRNA complexes in HEPES BPS buffer was introduced and followed by introducing a mixture of the Cas-12a: crRNA complexes and DETECTR probes. The Cas-12a: crRNA complexes used in this experiment were a mixture of Cas-12a proteins, each comprises a crRNA selected from the crRNAs (Alt-RR) listed in the following table. Alternatively, a Cas-12a: crRNA complexes using 1, 2, 3, 5, or 7 of the crRNAs can be used.

After that, the sequestration pens were again sealed by fluorinated oil. Images were taken to detect fluorescent signals (post-assay signals) associated with the probe. The signals generated by activated Cas-12a due to crRNA hybridization with the payload were obtained by deducting the post-assay signals with the pre-assay signals detected in each sequestration pen. The sequences of the probe and the crRNA are listed in table below

Table: DETECTR crRNA and probe

| | |
|---|---|
| Probe (SEQ ID NO.: 04) | TTATTATT |
| crRNA#1 (SEQ ID NO.: 05) | UAAUUUCUACUAAGUGUAGAUGAGGGCGAUGCCAC |
| | CUACGGC |
| crRNA#2 (SEQ ID NO.: 06) | UAAUUUCUACUAAGUGUAGAUUAGCCUUCGGGCAU |
| | GGCGGAC |
| crRNA#3 (SEQ ID NO.: 07) | UAAUUUCUACUAAGUGUAGAUCGUGCAGCUCGCCG |
| | ACCACUA |
| crRNA#4 (SEQ ID NO.: 08) | UAAUUUCUACUAAGUGUAGAUAUCGCGCUUCUCGU |
| | UGGGGUC |
| crRNA#5 (SEQ ID NO.: 09) | UAAUUUCUACUAAGUGUAGAUGUAGUUAAUGAUUA |
| | ACCCGCC |
| crRNA#6 (SEQ ID NO.: 10) | UAAUUUCUACUAAGUGUAGAUCUCAGGGCGGACUG |
| | GGUGCUC |
| crRNA#7 (SEQ ID NO.: 11) | UAAUUUCUACUAAGUGUAGAUCGUCGCCGUCCAGC |
| | UCGACCA |
| crRNA#8 (SEQ ID NO.: 12) | UAAUUUCUACUAAGUGUAGAUCAACAGCCACAAGG |
| | UCUAUAU |
| crRNA#9 (SEQ ID NO.: 13) | UAAUUUCUACUAAGUGUAGAUGAUGUUGUGGCGGG |
| | UCUUGAA |
| crRNA#10 (SEQ ID NO.: 14) | UAAUUUCUACUAAGUGUAGAUGUCGCCCGGCCUCA |
| | GUGAGCG |

The DETECTR signal (the difference between the pre-assay signal and the post-assay signal) of each sequestration pen was plotted against the signal of GFP in FIG. 16. In FIG. 16, each dot represents a DETECTR signal read-out of a sequestration pen, and the pentagon dots were sequestration pens selected for off-chip qPCR verification. Those sequestration pens were selected as the cells therein exhibiting high DETECTR signals. Nevertheless, the one sequestration pen at the left bottom corner exhibited low DETECTR signal and low GFP signal was selected as a control.

The arrows in FIG. 16 point to the sequestration pens that were verified to have high copy number of the payload by qPCR, indicating the payload was replicated well in those cells. Accordingly, the result shows that cells exhibiting higher DETECTR signals were highly possible to be GFP+ cells. In other words, based on the DETECTR signals, a group of virus-producing cells with the better producers enriched can be obtained. The experiment successfully adapted DETECTR assay in the microfluidic device for screening better producer cells.

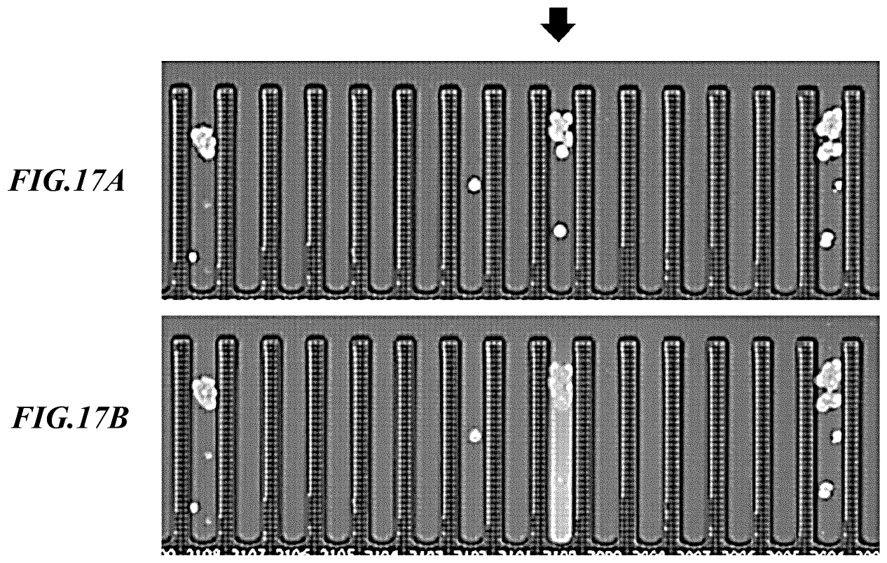

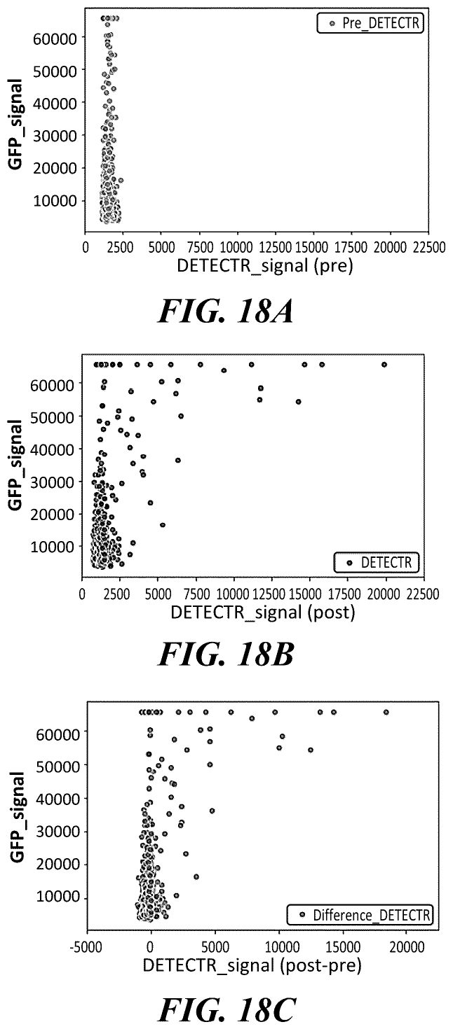

Alternative. The TA-rich probe (SEQ ID NO.: 04) was placed with a C rich probe (SEQ ID NO.: 15: CCCCCCCC). FIG. 17A and FIG. 17B shows an image of the beginning of the DECTECTR assay and an image after 10 minutes of the DECTECTR assay respectively. The arrowed chamber exhibited significant increase of signal associated with the probe indicating a high concentration of payload presence. Moreover, by performing the NEM treatment and the C rich probe, the DETECTR pre-assay signals were more uniformly distributed at lower level (FIG. 18A), and the post-assay signals between each sequestration pen were more distinguishable (FIG. 18B) and so were the DETECTR signals calculated by subtracting post-assay signal with pre-assay signal (FIG. 18C).

### Example 3: Evaluating Production of a Virus-Producing Cell Using Capture Beads

This experiment was conducted using capture beads to capture the viral particles produced by the producer cells to evaluate the amount of production. The viral vectors encapsulated in the viral particles were evaluated by de-stabilizing the viral particles and staining the viral vector with DNA dye.

Capture bead and Cell loading. A microfluidic device was prepared as described above. The microfluidic device comprises a plurality of chamber (i.e., sequestration pens). Each chamber was disposed with a single untransfected HEK293 cell or a single transfected HEK293 cell (triple-plasmid transfection to produce AAV9 particles).

Before disposing the cells, a fluidic medium comprising AAV capture beads was introduced in the flow region, and each sequestration pen was disposed with a single capture bead. The capture beads were streptavidin beads (Spherotech #SVP-150-4) coated with biotinylated anti-AAV9 antibodies (CaptureSelect Biotin Anti-AAV9 conjugate, ThermoFisher®). Then, a fluidic medium comprising untransfected cells was introduced, and single untransfected cells were disposed into each sequestration pens per-selected for untransfected cells. Another fluidic medium comprising transfected cells (i.e., cells being transfected with AAV9 particles) was introduced, and single transfected cells were disposed into each sequestration pens per-selected for transfected cells. Cells were cultured on chip for 72 hours. A physical titer assay as described below was performed at the end of every day (i.e., every 24 hours).

Physical titer assay. Baseline images were taken before the assay was conducted. A fluidic medium comprising CF660R-labeled anti-AAV9 antibodies was introduced and perfused for 60 minutes to let the anti-AAV9 antibodies diffuse into the sequestration pens. A culture medium was then perfused to flush out the remaining anti-AAV9 antibodies in the flow region. Images were taken to detect signals associated with the CF660R labeling.

Cell Lysis and mRNA capture. After 72 hours, mRNA capture beads of OptoSeq™ Single Cell 3′ mRNA Kit (Berkeley Lights, Inc.) were introduced and disposed in each sequestration pen. The mRNA capture beads were positioned close to the opening of the sequestration pen to the flow region, while the AAV capture beads were positioned to the distal end of the sequestration pen. Then, a lysis buffer of the kit was introduced into the microfluidic device to lyse the cells cultured in the sequestration pens. Then, a viscosity gradient flush was performed by introducing post-lysis buffer comprising decreasing concentration of glycerol. The viscosity gradient flush removed the remaining lysis buffer on chip while minimizing the risk of flushing away the mRNA capture beads in the chambers. Then, reverse transcription and barcode generation were performed on chip using OptoSeq Single Cell 3′ mRNA kit (Berkeley Lights, Inc.). The mRNA capture beads were unloaded using laser-generated bubble dislodge and collected. The microfluidic device was then treated with benzonase before other assays were conducted. After that, another physical titer assay (post-lysis) was performed according to the description thereof.

Genomic titer assay. After the post-lysis physical titer assay, in order to de-stablize the AAV particles, the culture condition of the microfluidic device was adjusted to low pH and high temperature. First of all, the temperature of the microfluidic device was set to 25 C degree. Then, the device was primed as described above. Bris-Tris (EDTA, pH 5.6) was then introduced into the microfluidic device and the temperature was set to 55 C degree. The culture condition was maintained for 2.5 hours before lowering down the temperature back to 25 C degree. Baseline images were taken. After that, a SYTO solution comprising SYTO24 and SYTO17 (ThermoFisher®) was introduced. After perfusion of the SYTO solution for over 60 minutes, the microfluidic device was washed by Bris-Tris (EDTA). Then, images were taken for detecting signals associated with the DNA dye.

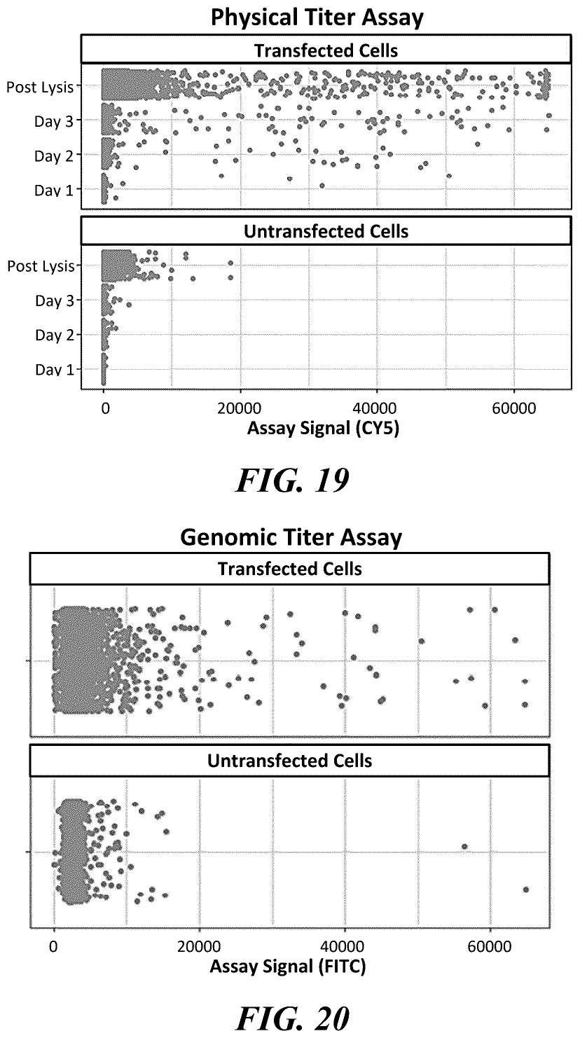

FIG. 19 shows the results of the physical titer assay. The transfected cells exhibited stronger signals and the signals increased over time. While the signal detected from the chamber disposed with untransfected cells also increased over time, the increasement was less significant compared to that of the transfected cells. That said, the method of the present disclosure can effectively identify cells that were transfected and producing the AAV9 particles.

FIG. 20 shows the results of the genomic titer assay. Overall, the transfected cells exhibited stronger signals than the untransfected cells. Together with the results of FIG. 19, the cells that were producing high amounts of AAV9 particles and high amounts of payload can be identified. Therefore, a group of candidate cells with better producers enriched can be collected for further analysis and verification.

### Example 4: Tetracycyline Inducible Expression System (Off-Chip)

In this example, three producer cell lines for AAV production (HEK293, cell lines 3D, 1B, and 2G) constructed with AAV helper genes controlled by a tetracycline response element (TRE) promoter were tested. The producer cell lines were also engineered to consecutively express reverse tetracycline-controlled transactivators (rtTA), which, upon binding with doxycycline can undergo conformational change and bind to the TRE thereby initiating the expression of helper genes. The productivity of the three cell lines was predetermined with cell line 3D the best producer among the three following cell line 1B and then cell line 2G. A parental cell line of the three producer cell lines was also included in this experiment as a negative control.

4A. Cells were maintained in Protein Expression Medium (PEM, cat. 12661-013, ThermoFisher®) before being treated with inducing agents, e.g., doxycycline. Alternatively, the cells were treated with a vector comprising a nucleic acid sequence of SEQ ID NO: 16, which encodes a tetracycline-controlled transactivator (tTA) having an amino acid sequence as set forth in SEQ ID NO: 17. The expressed tTA was configured to bind to the TRE and initiate the helper genes expression thereby bypassing the need of using doxycycline for induction. The vectors tested in this example include plasmid and adeno-associated virus (AAV).

Doxycycline. Doxycycline was purchased from Sigma (D3072). An induction solution having doxycycline at a concentration of 2 μg/ml in PEM medium was prepared. Then, 100 ul of induction solution was mixed with 100 ul of cell suspension (1E6 cells/ml). After induction, cells were maintained in a 96-well plate for recovery and then collected for rtPCR analysis targeting AAV genome to examine AAV production.

Plasmid induction. A plasmid (pTetoff, 7140 bp) was designed and constructed for expressing tTA within the producer cells upon transfection. The plasmid comprises a cytomegalovirus (CMV) promoter, which is operably linked to the nucleic acid sequence of SEQ ID NO: 16. In this experiment, 1 μg of the plasmid was firstly mixed with 0.3 ul of Xfect transfection reagent (TaKaRa®) and then mixed with FreeStyle™ 293 Expression medium. 50 ul of the mixture was then mixed with 50 ul of cell suspension (2E6 cells/ml). After induction, the cells were maintained in a 96-well plate for four hours. After that, 150 ul of fresh PEM medium was added. Cells were then collected to detect the AAV genome using rtPCR. The Ct value was used as an indication of AAV production.

AAV vector induction. The AAV vectors used in this experiment were produced by vendor (VirusBuilder®) using the plasmid designed in house. The AAV vector was designed to carry an expression cassette comprising the nucleic acid sequence of SEQ ID NO: 16, operably linked to a EFIA promoter. The AAV vectors (1E8 viral particles in 100 ul of PEM medium) were mixed with 100 ul of cell suspension (1E6 cells/ml) for induction. After induciton, the cells were maintained in a 96-well culture plate and then collected to detect the AAV genome using rtPCR. The Ct value was used as an indication of AAV production.

4B. In this example, the cells were either treated with doxycycline or AdV vectors, designed for expressing tTA to bypass doxycycline induction. Doxycycline treatment was conducted following the same procedure as described in 4A except that, in this experiment, 1 ml of cell suspension (1E6 cells/ml) was mixed with 1 ml of doxycycline solution. After induction, cells were maintained in a 6-well culture plate and collected for rtPCR analysis.

Adl′ vector induction. The AdV vectors used in this experiment were produced by vendor (VirusBuilder®) using the plasmid designed in house. The AdV vector was designed to carry an expression cassette comprising the nucleic acid sequence of SEQ ID NO: 16 operably linked to a CBh promoter and a nucleic acid encoding mCherry operably linked to a mPGK promoter. The AdV vectors (2E6 viral particles in 1 ml of PEM medium) were mixed with 1 ml of cells suspension (1E6 cells/ml). After induction, the cells were maintained in a 6-well culture plate and then collected for rtPCR analysis. Signal associated with mCherry was also detected for determining the success of transfection (data not shown).

4C. In this experiment, a plasmid (psctTA2, 8958 bp) was designed to carry a nucleic acid sequence of SEQ ID NO: 18 encoding a single chain tetracycline-controlled transactivator (sctTA) having an amino acid sequence as set forth in SEQ ID NO: 19. The nucleic acid sequence encoding the sctTA was operably linked, at 5′ thereof, to a CMV promoter. The plasmid also comprises a nucleic acid sequence encoding mCherry protein operably linked to the CMV promoter. The plasmid psctTA2 (1 ug) was firstly mixed with 0.3 ul of Xfect transfection reagent (TaKaRa®) and then mixed with FreeStyle™ 293 Expression medium. 50 ul of the mixture was then mixed with 50 ul of cell suspension (2E6 cells/ml). After induciton, the cells were maintained in a 96-well plate for four hours. After that, 150 ul of fresh PEM medium was added. Cells were then collected to detect the AAV genome using rtPCR. The plasmid pTetoff described in Experiment 4A was also tested in this experiment for comparison.

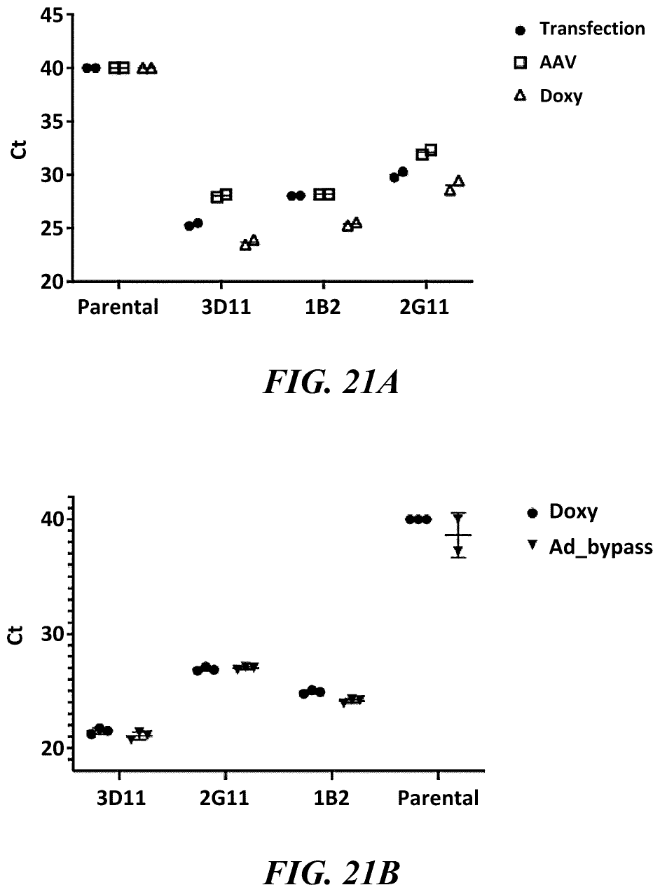

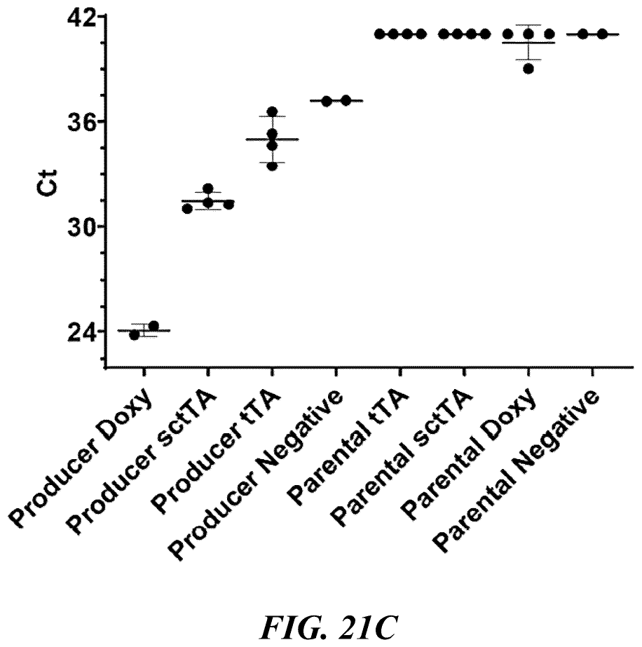

FIG. 21A shows the results of Experiment 4A, and FIG. 21B shows the result of Experiment 4B. The lower Ct value indicates higher amount of AAV genome, meaning higher AAV production. It is noted that all the groups treated with inducing agents have a Ct value lower than the negative control (Parental cells). Cell line 3D had the lowest Ct value (about 23 to 29 in FIG. 21A, and about 20 to 22 in FIG. 21B)) among the three cell lines in all induction methods followed by cell line 1B (around 26 to 29 in FIG. 21A, and about 25 in FIG. 21B) and cell line 2G (around 29 to 34, and about 27 in FIG. 21B). These results were consistent with the predetermined productivity of the three cell lines. In sum, the experiments verified the induction methods were able to induce the AAV production in the producer cell lines.

FIG. 21C shows the results of Experiment 4C. The cells of Cell line 3D induced with doxycycline had the lowest Ct indicating the highest production among the groups. The cells exhibit better (lower Ct) production when being induced by plasmids carrying a nucleotide acid encoding sctTA compared to tTA. The results support the hypothesis that expressing sctTA can avoid forming heterodimers with rtTA and therefore obtain better induction efficiency.

### Example 5: Tetracycline inducible expression system (on-chip)

In this example, producer cell lines engineered with tetracycline inducible expression system were tested in a microfluidic device (on-chip). The producer cell lines include cell lines 3D, 1B, and 2G, which are described in Example 4, and a parental cell line was included as negative contorl. Microfluidic devices (i.e., chips) were prepared according to the Priming Regime as described above.

Cell penning. Cells were cultured in PEM medium and cell suspensions (about 2E6 cells/ml, the viability of each cell line was around 95% to 98%) thereof were prepared for loading before the experiment. The cell suspensions of producer cell lines were then introduced respectively, and single cells of each producer cell line were disposed, using DEP or gravity, into respective chambers of pre-assigned regions of the microfluidic device (Day 1). After that, cells were cultured within respective chambers in the microfluidic device for 96 hours. Images were taken to observe cell growth in the microfluidic device (images not shown).

Capture bead loading. The capture beads used in the experiment were streptavidin beads (Spherotech #SVP-150-4) coated with biotinylated anti-AAV8 antibodies (CaptureSelect Biotin Anti-AAV8 conjugate, ThermoFisher®). A fluidic medium comprising the capture beads was introduced in the flow region of the microfluidic device, and each chamber was then disposed with a single capture bead. After bead loading, cells were maintained together with the capture beads within respective chambers in the microfluidic device before the next step.

Hydrogel capping. The prepolymer composition configured to form a reversible hydrogel barrier as describe above was prepared. The prepolymer composition was introduced into the flow region of the microfluidic device and allowed to diffuse into the chambers. Then, solidification of the prepolymer composition was photo-activated within every other chamber at an area close to the opening thereof thereby forming reversible hydrogel caps in-situ. The capped chambers were therefore served as a preserving chamber, and the uncapped chambers were the assay chambers as described herein.

5A-1. In Experiment 5A-1, four chips were prepared. Among them, one was used to perfrom doxycycline induction, two of them were used to perfrom AdV vector induction (high MOI and low MOI), and the last chip was a negative control. The inductions were all conducted on Day 2.

Doxycycline induction. A doxycycline solution was prepared by mixing doxycycline (1 ug/ml) and KCl (60 mM) in culture media. The doxycycline solution was introduced into the flow region of the microfluidic device, and the doxycycline was allowed to diffuse into the chamber for induction. The induction was continued overnight, and cells were maintained on chip for recovery. Then, a physical titer assay as described in Example 3 was conducted on Day 5 using CaptureSelect™ Alexa Fluor™ 647 Anti-AAVX Conjugate (ThermoFisher® 7233522100, 0.5 mg/mL Stock). Images were taken to detect signals associated with mCherry, which indicates success of transduction (In doxycycline induction, the induction was conducted using free doxycycline, so there should be no signal).

AdV′ vector induction. AdV vectors were prepared by a vendor as described in Example 4 for preparing an induction solution (2E7 viral particles/ml). The induction solution was diluted into two working solutions of different concentration: MOI 100 (High MOI) and MOI 50 (Low MOI). The two working solutions were then introduced into respective chips. The induction was continued overnight, and cells were maintained on chip for recovery. Then, a physical titer assay as described in Example 3 was conducted on Day 5 using CaptureSelect™ Alexa Fluor™ 647 Anti-AAVX Conjugate (ThermoFisher® 7233522100, 0.5 mg/mL Stock). Images were taken to detect signals associated with mCherry, which indicates success of transduction.

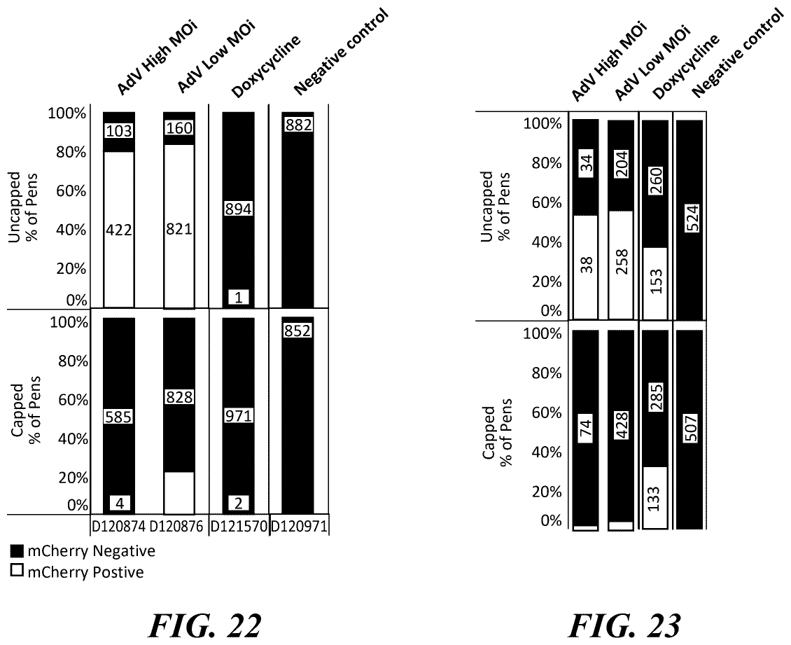

Results. FIG. 22 shows the fraction of mCherry positive chambers of the four chips tested in Experiment 4A. On AdV high MOI chip and AdV low MOI chip, signals associated with mCherry can be detected within around 80% of the uncapped chambers (422 positive v. 103 negative on high MOI AdV, and 821 positive v. 160 negative on low MOI AdV). Despite that there were some false positive signals on the AdV low MOI chip, only a small number of capped chambers were mCherry positive. The results showed that the hydrogel cap was able to effectively block AdV vectors from entering the preserving chambers. On the other hand, basically all chambers on the doxycycline induction chip were mCherry negative with an ignorable number of false positive chambers. This result aligns with the experiment design because the doxycycline induction chip performed induction using doxycycline but not vectors and should not exhibit any mCherry signals.

FIG. 23 shows the fraction of capsid positive chambers according to the physical titer assay conducted on Day 3 after induction. Around 55% to 60% uncapped chambers of the AdV high MOI chip (38 positive v. 34 negative) and the AdV low MOI chip (258 positive v. 204 negative) were capsid positive, which were higher than the uncapped chambers of doxycycline induction chip (153 positive v. 260 negative). This result showed that the AdV vector had better induction efficiency than free doxycycline. Besides, as the hydrogel cap effectively blocked AdV vectors from entering the preserving chambers, only an ignorable number of capped chambers of AdV induction chips were capsid positive. A similar fraction of capsid positive chambers was detected between capped and uncapped chambers of the doxycycline induction chip. This result was also consistent with the experiment design because the doxycycline was too small to be blocked by the hydrogel caps used in this experiment.

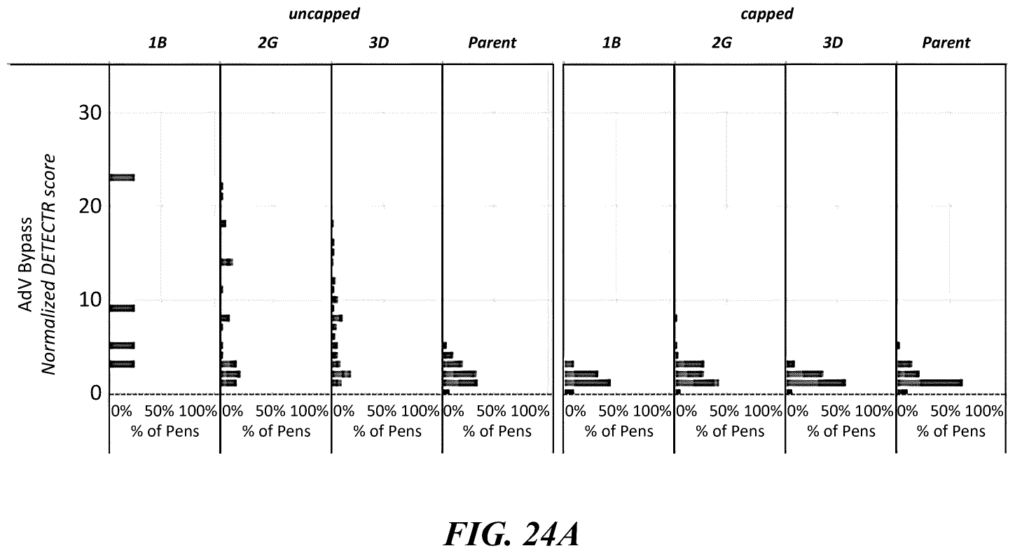

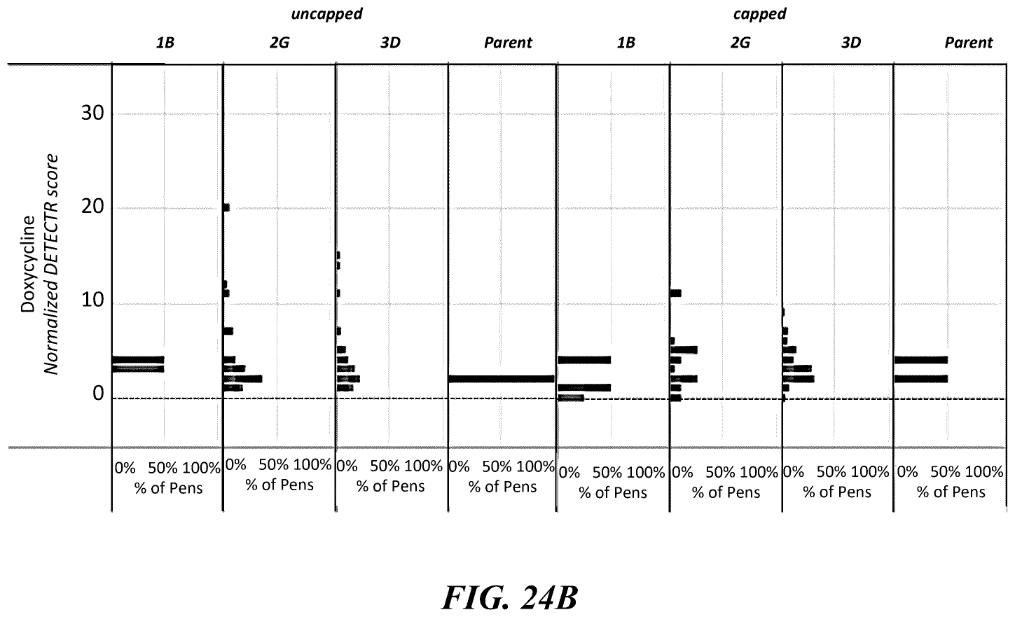

5A-2. Alternatively, in Experiment 5A-2, the Experiment 5A-1 was repeated, and a DETECTR assay was performed to determine the productivity of the tested producer cell lines. The DETECTR assay was conducted accoridng to Example 2 but using a mixture of crRNAs listed in the following table. Cell numbers of each chamber were also determined for normalizing the signals detected in the DETECTR assay. The histograms of FIG. 24A and FIG. 24B show normalized DETECTR assay score in y-axis and the percentage of pens (i.e., chambers) in x-axis. First of all, as shown in FIG. 24A, all capped pens exhibited lower scores compared to the uncapped pens, indicating that the hydrogel cap was efficient to block the AdV vectors from entering the preserving chambers. While the difference between the capped and uncapped pens was not as obvious in FIG. 24B, this was because doxycycline was able to pass through the hydrogel caps used in this experiment.

Secondly, all producer cell lines exhibited higher score than the parental cell line no matter they were induced by the AdV vectors or doxycycline. This result confirmed that the AdV vectors were able to induce the helper gene expression resulting in viral particle production to bypass the need of doxycycline. In fact, the cells induced by AdV vectors had higher score than the cells induced by doxycycline, indicating AdV vectors can induce the TRE promoter better than doxycycline. This finding was consistent with the results in Experiment 5A-1.

Table: DETECTR crRNA

| | |
|---|---|
| crRNA#1 (SEQ ID NO: 20) | CCGTGTTCCAGCACGACGACT |
| crRNA#2 (SEQ ID NO: 21) | TCCCGGTCGAAGCTCTCGGGC |
| crRNA#3 (SEQ ID NO: 22) | TGCAGCTGCTCGCCGGCGGTC |
| crRNA#4 (SEQ ID NO: 23) | CCGACGCACATATCGAGGTGG |
| crRNA#5 (SEQ ID NO: 24) | GGGTCGTGCTCATGTACCGCT |
| crRNA#6 (SEQ ID NO: 25) | GCCCGACGACGATGCCGGCGA |
| crRNA#7 (SEQ ID NO: 26) | GGACGCCCGCAAGATCCGCGA |
| crRNA#8 (SEQ ID NO: 27) | CGGCGCCCCGCCGCTGGCGAT |
| crRNA#9 (SEQ ID NO: 28) | GGCGTAGCCCTACCGCACCGC |
| crRNA#10 (SEQ ID NO: 29) | CGCGGCGAGCTGTGCGTCCGT |

It is also noted that, among the chambers induced with AdV vectors (FIG. 24A), the capped chambers (i.e., preserving chambers) showed overall lower DETECTR scores than that of the uncapped chambers. This is because the hydrogel caps blocked the AdV vectors from entering the chambers, therefore, the cells within the capped chambers were not induced. In comparison, there was no significant difference between the capped chambers and the uncapped chambers induced by using doxycycline, because doxycycline was small enough to pass through the hydrogel caps.

5B. In this experiment, four chips were prepared. Among them, one was used to perfrom doxycycline induction, two of them were used to perfrom AAV vector induction (high MOI and low MOI), and the last one was a negative control. The cells were disposed on Day 1, and the induction was conducted on Day 3. Only cell lines 1B, 3D, and parent cell line were tested in this experiment.

Doxycycline induction. A doxycycline solution was prepared by mixing doxycycline (1 μg/ml) and KCl (60 mM) in culture media. The doxycycline solution was introduced into one of the four chips. The induction was continued overnight, and cells were maintained on chip for recovery. Then, a physical titer assay as described in Example 3 was conducted on each day of Day 3 to 5 (Only the data of Day 3 and Day 5 are shown here) after induction using CaptureSelect™ Alexa Fluor™ 647 Anti-AAVX Conjugate (ThermoFisher® 7233522100, 0.5 mg/mL Stock).

AAV vector induction. AAV vectors were prepared by a vendor as described in Example 4. Two AAV induction solutions were prepared comprising 500 MOI (low MOI) and 4000 MOI (high MOI) respectively. 50 ul of the induction solution was then mixed with 50 ul of culture media, and the mixtures were introduced into the flow region of designated chips. The induction was continued overnight, and cells were maintained on chip for recovery. Then, a physical titer assay as described in Example 3 was conducted on each day of Day 3 to 5 (Only the data of Day 3 and Day 5 are shown here) after induction using CaptureSelect™ Alexa Fluor™ 647 Anti-AAVX Conjugate (ThermoFisher® 7233522100, 0.5 mg/mL Stock).

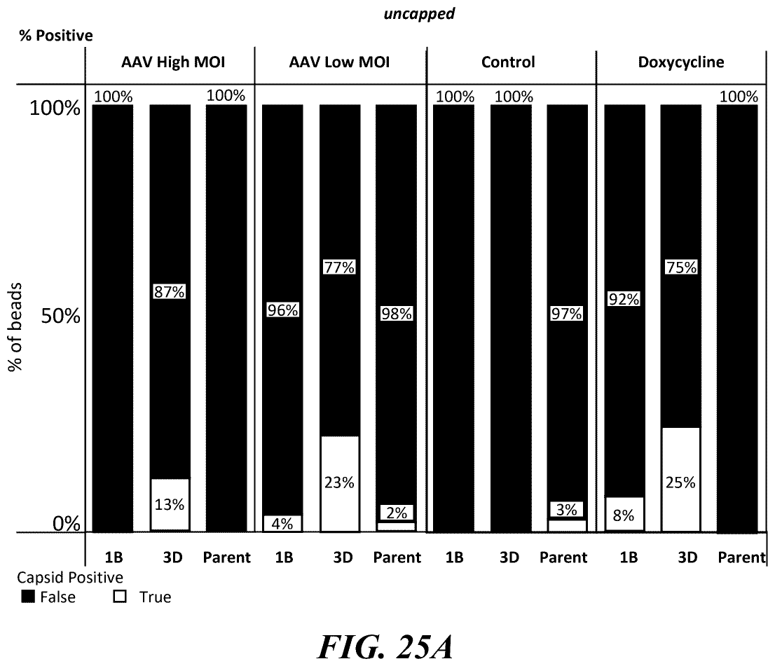

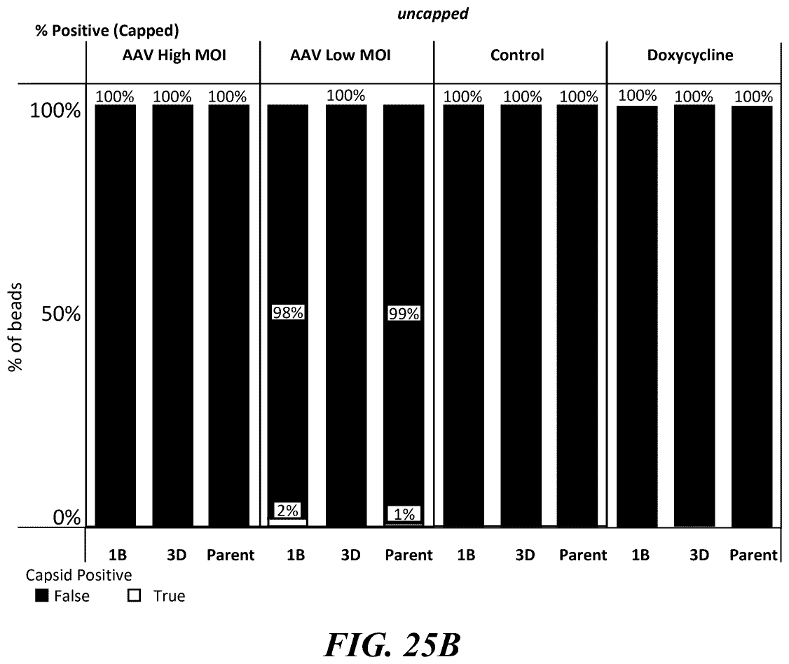

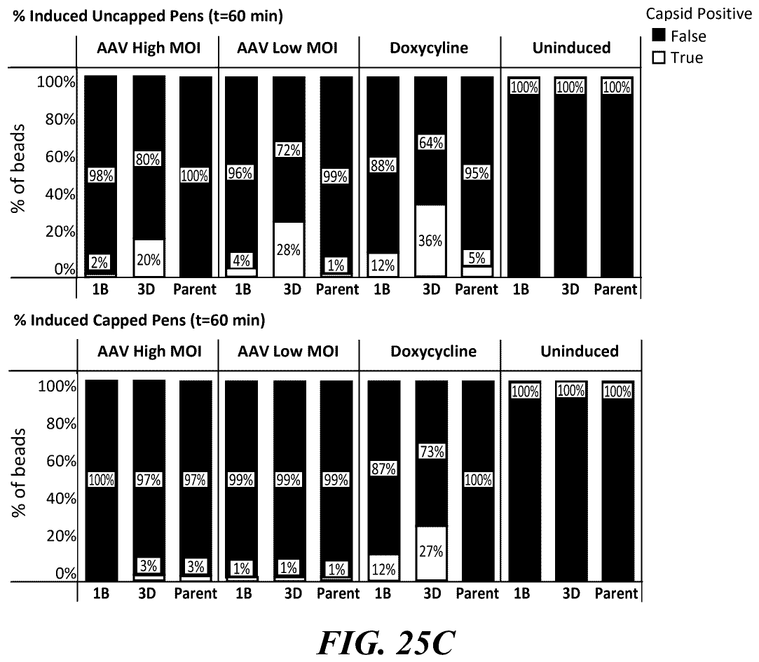

Results. The results show that the AAV vectors were able to induce the viral particle production (FIG. 25A, FIG. 25C), and the hydrogel caps effectively blocked the entrance of the AAV vectors but not free doxycycline (FIG. 25B, FIG. 25C). Among the chambers with cell line 3D of microfluidic devices induced by using high MOI AAV vectors, low MOI AAV vectors, doxycycline, and uninduced, at Day 3 (FIG. 25A) the fraction of positive beads were 13%, 23%, 25%, 0%, respectively. In comparison, the fraction of positive beads of cell line 1B were 0%, 4%, 8%, 0%, respectively. That said, cell line 3D exhibited higher signals than that of cell line 1B, which was consistent with the predetermined ranking that cell line 3D has higher productivity than cell line 1B.

Furthermore, the fraction of positive increased from Day 3 to Day 5. At Day 5 (FIG. 25C), the fraction of cell line 3D and cell line 1B were 20%, 28%, 36%, 0% and 2%, 4%, 12%, 0% respectively. This finding suggested that the producer cells can be maintained robustly on the microfluidic device, and continuing culturing the cells after induction can obtain more amount of the viral particles.

5C. In this experiment, producer cell line 3D and parental cell line were used to test induction by using doxycycline and plasmids carrying a nucleic acid encoding tTA. The cells were disposed on Day 1, and the induction was conducted on Day 4.

Doxycycline induction. A doxycycline solution was prepared by mixing doxycycline (1 μg/ml) and KCl (60 mM) in culture media. The doxycycline solution was introduced into one of the four chips. The induction was continued overnight, and cells were maintained on chip for recovery. Then, a physical titer assay as described in Example 3 was conducted on Day 3 and 4 after induction (only the data of Day 3 is shown here) using CaptureSelect™ Alexa Fluor™ 647 Anti-AAVX Conjugate (ThermoFisher® 7233522100, 0.5 mg/mL Stock). Images were taken to detect signals associated with mCherry, which indicates success of transduction (on doxycycline induction chip, there should be no mCherry signal).

Plasmid induction. Plasmids pTetoff carrying a nucleic acid encoding tTA as described in Example 4 were prepared. I ug of the plasmid pTetoff was firstly mixed with 0.3 ul of Xfect transfection reagent (TaKaRa®) and then mixed plasmids pTREmCherry-CMVGFP, which was used as an indicator of successful transfection in this experiment, in the FreeStyle™ 293 Expression medium. The plasmid mixture was then introduced into the flow region of the chip. After introducing the plasmid mixture, the chip was centrifuged for 2 minutes to facilitate disposing the plasmids into the chambers. Then, the introduction of plasmid mixture and centrifugation of the chip were repeated once (i.e., double import of the plasmid mixture). The induction was continued overnight. Then, a physical titer assay as described in Example 3 was conducted on Day 3 and 4 after induction (only the data of Day 3 is shown here) using CaptureSelect™ Alexa Fluor™ 647 Anti-AAVX Conjugate (ThermoFisher® 7233522100, 0.5 mg/mL Stock). Images were taken to detect signals associated with mCherry, which indicates success of transduction.

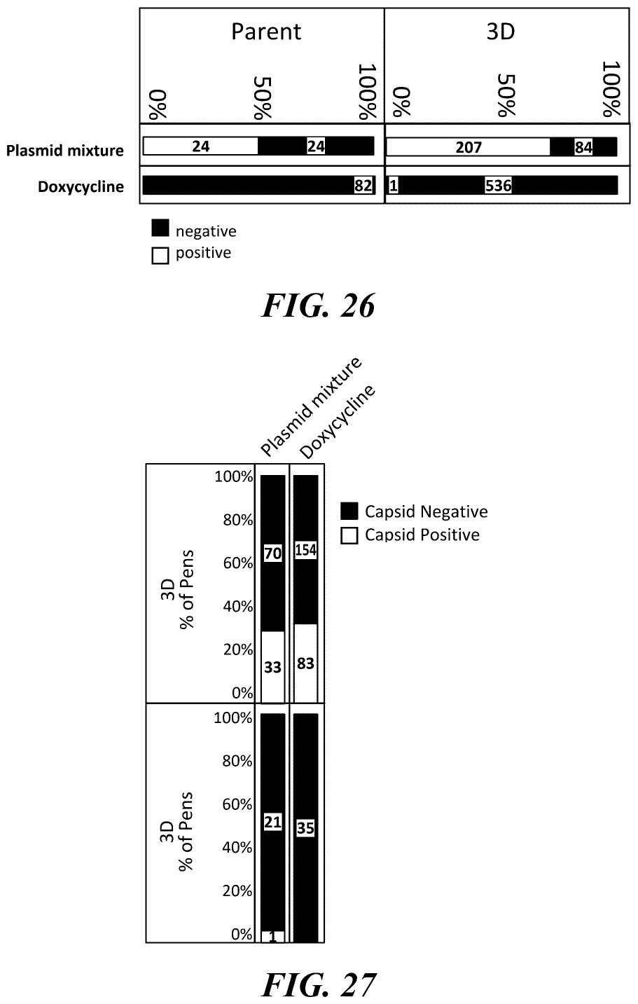

Results. FIG. 26 shows the fraction of mCherry positive and negative chambers observed on respective chips treated with doxycycline and plasmid mixture of this experiment. It is noted that the plasmids were able to transfect the cells, and the mCherry was able to be expressed by the transfected cells within the chambers. There were 24 positive chambers and 24 negative chambers of the parental cell line, and 207 positive chambers and 84 negative chambers of cell line 3D. In comparison, the chip treated with doxycycline contained nearly zero chamber (with only one false positive chamber) exhibiting mCherry signal.

FIG. 27 shows the results of the physical titer assay. Around 30% of the chambers (33 out of 103) culturing producer cell line 3D on the chip treated with the plasmid mixture were capsid positive, indicating viral particle production. The percentage was similar to the capsid positive chambers of cell line 3D induced with doxycycline (83 out of 237). In comparison, chambers of parental cell line were all capsid negative (except for one false positive) no matter being treated with the plasmid mixture of doxycycline. The results of FIG. 26 and FIG. 27 verified that the plasmid carrying nucleic acid encoding tTA was able to transfect cells and induce viral particle production on chip.

### PARTIAL LISTING OF EMBODIMENTS

Embodiment 1: A method for preserving a subset of biological micro-objects within a microfluidic device, comprising: moving a first subset of a plurality of biological micro-objects disposed in a first chamber of a microfluidic device into a second chamber of the microfluidic device, wherein the microfluidic device comprises a microfluidic circuit material defining a flow region and a plurality of chambers fluidically connecting to the flow region, wherein the plurality of chambers comprising the first chamber and the second chamber; designating one of the first chamber and the second chamber as a preserving chamber and the other as an assay chamber; and forming a first in situ-generated cap within the preserving chamber, wherein the first in situ-generated cap comprises a porosity to selectively block passage of the biological micro-objects between the preserving chamber and the flow region.

Embodiment 2: The method of embodiment 1, before moving the first subset of the plurality of biological micro-objects, the method further comprises: disposing a biological micro-object into the first chamber; and expanding the biological micro-object into the plurality of biological micro-objects.

Embodiment 3: The method of embodiment 1 or 2, wherein moving the first subset of the plurality of biological micro-objects into the second chamber comprises: selecting one or more biological micro-object(s) from the plurality of biological micro-objects in the first chamber; and moving the selected one or more biological micro-object(s) from the first chamber into the second chamber, thereby forming a first subset of the plurality of biological micro-objects in the second chamber.

Embodiment 4: The method of any one of embodiments 1 to 3, wherein moving the first subset of the plurality of biological micro-objects into the second chamber comprises: moving one or more biological micro-object(s) from the first chamber into a transit area within the flow region and from the transit area into the second chamber.

Embodiment 5: The method of embodiment 4, wherein the transit area is substantially enclosed by an in situ-generated barrier, thereby preventing the one or more biological micro-object(s) from entering an area of the flow region other than the transit area while allowing the one or more micro-object(s) to access the transit area, the first chamber, and the second chamber.

Embodiment 6: The method of embodiment 4 or 5, wherein the first chamber comprises a first opening to the flow region and the second chamber comprises a second opening to the flow region, and wherein the method further comprises forming an in situ-generated separating element at an area within the transit area between the first opening and the second opening.

Embodiment 7: The method of embodiment 5 or 6, further comprising removing the in situ-generated barrier after moving the first subset of the plurality of micro-objects into the second chamber.

Embodiment 8: The method of any one of embodiments 1 to 7, wherein moving the first subset of the plurality of biological micro-objects into the second chamber comprises using dielectrophoresis (DEP) force, gravity, centrifugation, or a combination thereof.

Embodiment 9: The method of any one of embodiments 1 to 8, wherein the plurality of biological micro-objects is a clonal population.

Embodiment 10: The method of any one of embodiments 1 to 9, further comprising culturing the first subset of the plurality of biological micro-objects in the second chamber.

Embodiment 11: The method of any one of embodiments 1 to 10, wherein moving a first subset of the plurality of biological micro-objects into the second chamber further comprises retaining a second subset of the plurality of biological micro-objects in the first chamber.

Embodiment 12: The method of embodiment 11, wherein the first subset and the second subset of biological micro-objects belong to a same clonal population.

Embodiment 13: The method of embodiment 11 or 12, further comprising culturing the second subset of the plurality of micro-objects in the first chamber.

Embodiment 14: The method of any one of embodiments 1 to 13, further comprising performing an assay in the assay chamber.

Embodiment 15: The method of embodiment 14, wherein the assay is performed after forming the first in situ-generated cap.

Embodiment 16: The method of embodiment 15, wherein the first in situ-generated cap within the preserving chamber prevents the assaying from being performed within the preserving chamber.

Embodiment 17: The method of any one of embodiments 14 to 16, wherein performing an assay in the assay chamber comprises: allowing the first subset of the plurality of biological micro-objects and/or the second subset of the plurality of biological micro-objects to produce a biological product of interest.

Embodiment 18: The method of embodiment 17, wherein performing an assay in the assay chamber further comprises assaying the biological product of interest.

Embodiment 19: The method of any one of embodiments 14 to 18, wherein performing an assay in the assay chamber comprises: introducing a lysis buffer into the flow region of the microfluidic device; diffusing the lysis buffer into the assay chamber; and blocking the lysis buffer from entering the preserving chamber with the first in situ-generated cap.

Embodiment 20: The method of any one of embodiments 1 to 19, wherein the first in situ-generated cap is moveably connected to one or more surface of the preserving chamber.

Embodiment 21: The method of any one of embodiments 1 to 20, wherein the first in situ-generated cap comprises a non-uniform thickness with respect to an axis of the chamber such that a portion of the in situ-generated cap is less thick than other portions thereof.

Embodiment 22: The method of any one of embodiments 1 to 21, wherein the first in situ-generated cap comprises a solidified polymer network.

Embodiment 23: The method of embodiment 22, wherein the solidified polymer network comprises a synthetic polymer, a modified synthetic polymer, a biological polymer, or any combination thereof.

Embodiment 24: The method of embodiment 22 or 23 wherein the solidified polymer network is reversible.

Embodiment 25: The method of any one of embodiments 1 to 24, wherein the biological micro-object is a cell.

Embodiment 26: A method of evaluating a virus-producing cell on a microfluidic device, the method comprises: culturing the virus-producing cell in a chamber of the microfluidic device, wherein the microfluidic device comprises a microfluidic circuit material defining a flow region and the chamber, such that the chamber opens to, and is fluidically connected to, the flow region; allowing the virus-producing cell to produce a viral particle; and evaluating a productivity of the virus-producing cell in producing the viral particle, and optionally, wherein a cell genetically identical to the virus-producing cell, is preserved on the microfluidic device, in a region other than the flow region or the chamber, while evaluating the productivity of the virus-producing cell.

Embodiment 27: The method of embodiment 26, wherein evaluating the productivity of the virus-producing cell comprises detecting the viral particle produced.

Embodiment 28: The method of embodiment 27, wherein detecting the produced viral particle comprises: disposing a virus-capturing structure within the chamber (or proximal to the opening of the chamber), wherein the virus-capturing structure comprises a capture moiety configured to capture the viral particle; introducing a reporter molecule into the flow region, wherein the reporter molecule comprises a detectable label and a binding component configured to bind the viral particle; and detecting a signal associated with the detectable label of the reporter molecule, and optionally, wherein the signal associated with the detectable label is detected within the chamber (or proximal to the opening of the chamber).

Embodiment 29: The method of embodiment 28, wherein the virus-capturing structure is a capture bead or an in situ-generated capture structure.

Embodiment 30: The method of embodiment 28 or 29, wherein the capture moiety and/or the binding component comprises a peptide or a protein.

Embodiment 31: The method of any one of embodiments 28 to 30, wherein the capture moiety and/or the binding component comprises an antibody, or a fragment thereof, configured to specifically bind to the viral particle.

Embodiment 32: The method of any one of embodiments 26 to 31, wherein evaluating the productivity of the virus-producing cell comprises detecting a payload of the produced viral particle.

Embodiment 33: The method of embodiment 32, wherein detecting a payload of the produced viral particle comprises introducing a nucleic acid dye into the flow region of the microfluidic device and detecting a signal associated with the nucleic acid dye, and optionally, wherein the signal associated with the nucleic acid dye is detected within the chamber (or proximal to the opening of the chamber).

Embodiment 34: The method of embodiment 33, wherein the nucleic acid dye is a DNA dye or a RNA dye.

Embodiment 35: The method of embodiment 34, wherein the nucleic acid dye is SYTO Green, QuantiFluor, GelGreen, or SYBR Gold.

Embodiment 36: The method of any one of embodiments 32 to 35, further comprising de-stabilizing the produced viral particle.

Embodiment 37: The method of embodiment 36, wherein de-stabilizing the produced viral particle is performed by heating the microfluidic device or a portion thereof comprising the chamber and/or a portion of the flow region proximal to the chamber, changing pH of media with the chamber and/or the portion of the flow region proximal to the chamber, or a combination thereof.

Embodiment 38: The method of any one of embodiments 32 to 37, further comprising introducing a nuclease into the microfluidic device, and optionally, wherein introducing the nuclease occurs prior to destabilizing the produced viral particle.

Embodiment 39: The method of any one of embodiments 32 to 38, wherein detecting the payload of the produced viral particle comprises amplifying the payload in the chamber.

Embodiment 40: The method of embodiment 39, wherein amplifying the payload comprises introducing a polymerase, a primer set recognizing the payload, and dNTPs into the microfluidic device.

Embodiment 41: The method of embodiment 40, wherein amplifying the payload further comprises introducing a recombinase.

Embodiment 42: The method of embodiment 40 or 41 wherein amplifying the payload further comprises introducing a crowding reagent, and wherein the crowding reagent is introduced separately from the polymerase and/or the primer set.

Embodiment 43: The method of any one of embodiments 39 to 42, wherein amplifying the payload further comprises introducing a nucleic acid dye, and further wherein detecting a payload of the viral particle comprises detecting a signal associated with the nucleic acid dye.

Embodiment 44: The method of any one of embodiments 39 to 42, wherein detecting the payload of the produced viral particle comprises: introducing an endonuclease and a first probe configured to recognize the payload, wherein: the first probe comprises a first detectable label and a quencher moiety configured to quench the first detectable label; the first probe is configured to recognize the payload, thereby forming a first probe: payload complex; and the endonuclease is configured to cleave the first probe: payload complex, thereby releasing the first detectable label from the first probe: payload complex; and detecting a signal associated with the first detectable label, thereby detecting the payload of the produced viral particle.

Embodiment 45: The method of any one of embodiments 32 to 42, wherein detecting a payload of the produced viral particle comprises: introducing an endonuclease: nucleic acid complex and a second probe comprising a second detectable label and a quencher configured to quench the second detectable label, wherein: the endonuclease: nucleic acid complex is configured to recognize the payload, thereby activating the endonuclease of the endonuclease: nucleic acid complex, and the activated endonuclease is configured to cleave the second detectable label from the second probe; and detecting a signal associated with the second detectable label.

Embodiment 46: The method of embodiment 45, wherein the endonuclease: nucleic acid complex comprises a nucleic acid comprising a first segment complementary to a segment of the payload and a second segment comprising a clustered regularly interspaced short palindromic repeats (CRISPR) sequence.

Embodiment 47: The method of embodiment 45 or 46, wherein detecting a payload of the produced viral particle comprises: introducing a first fluidic medium comprising the endonuclease: nucleic acid complex into the microfluidic device; and introducing a second fluidic medium comprising the second probe into the microfluidic device.

Embodiment 48: The method of embodiment 47, wherein detecting a payload of the produced viral particle comprises, after introducing the second fluidic medium, detecting a post-assay signal associated with the second detectable label.

Embodiment 49: The method of embodiment 47 or 48, wherein detecting a payload of the produced viral particle further comprises, before introducing the first fluidic medium and the second fluidic medium, introducing a third fluidic medium comprising the second probe into the microfluidic device and detecting a pre-assay signal associated with the second detectable label.

Embodiment 50: The method of embodiment 49, wherein evaluating a productivity of the virus-producing cell comprises determining a difference between the pre-assay signal and the post-assay signal.

Embodiment 51: The method of any one of embodiments 45 to 50, wherein the endonuclease is a CRISPR-associated (Cas) protein.

Embodiment 52: The method of embodiment 51, the Cas protein comprises Cas-12a, Cas-13, Cas-13a, Csm6, or a mixture thereof.

Embodiment 53: The method of embodiment 51 or claim 52, wherein the second probe is an A/T rich probe or a C rich probe.

Embodiment 54: The method of embodiment 53, wherein the second probe is a C rich probe comprising SEQ ID NO.: 15.

Embodiment 55: The method of any one of embodiments 45 to 54 wherein the second detectable label is a fluorophore.

Embodiment 56: The method of any one of embodiments 45 to 55, further comprising treating the chamber with a nuclease inhibitor.

Embodiment 57: The method of embodiment 56, wherein the nuclease inhibitor is a proteinase.

Embodiment 58: The method of any one of embodiments 26 to 57, wherein evaluating the productivity of the virus-producing cell further comprises capping the chamber, thereby substantially isolating the chamber from the flow region.

Embodiment 59: The method of embodiment 58, wherein the capping comprises introducing a water immiscible fluidic medium into the flow region of the microfluidic device.

Embodiment 60: The method of embodiment 59, wherein the water immiscible fluidic medium comprises an alkane, a fluoroalkane, an oil, a hydrophobic polymer, or any combination thereof.

Embodiment 61: The method of embodiment 58, wherein the capping comprises introducing air into the flow region of the microfluidic device, thereby substantially replacing liquid in the flow region with air.

Embodiment 62: The method of any one of embodiments 26 to 61, wherein culturing the virus-producing cell further comprises introducing a fluidic medium comprising the virus-producing cell into the flow region and disposing the virus-producing cell into the chamber.

Embodiment 63: The method of any one of embodiments 26 to 62, wherein allowing the virus-producing cell to produce a viral particle comprises inducing the virus-producing cell to produce the viral particle.

Embodiment 64: The method of embodiment 63, wherein the virus-producing cell comprises a helper gene of the viral particle, and expression of the helper gene is controlled by an inducible promoter; and wherein inducing the virus-producing cell to produce the viral particle comprises inducing the inducible promoter.

Embodiment 65: The method of embodiment 64, wherein inducing the inducible promoter comprises contacting the virus-producing cell with an inducing agent.

Embodiment 66: The method of embodiment 65, wherein contacting the virus-producing cell with the inducing agent comprises: introducing a fluidic medium comprising the inducing agent; and allowing the inducing agent to diffuse into the chamber and contact the virus-producing cell, or disposing the inducing agent into the chamber such that it can contact the virus-producing cell.

Embodiment 67: The method of embodiment 65 or 66, wherein the inducing agent comprises a small molecule inducer configured to induce the inducible promoter.

Embodiment 68: The method of embodiment 67, wherein the small molecule inducer is configured to form a complex with a transactivator, which is expressed by the producer cell, and wherein the inducer-transactivator complex is configured to bind to the inducible promoter, thereby inducing the inducible promoter.

Embodiment 69: The method of embodiment 68, wherein the producer cell is engineered to consecutively express the transactivator.

Embodiment 70: The method of any one of embodiments 67 to 69, wherein the inducible promoter comprises a tetracycline response element (TRE), and wherein the small molecule inducer is tetracycline, and the transactivator is a reverse tetracycline-controlled transactivator (rtTA).

Embodiment 71: The method of embodiment 70, wherein the tetracycline is doxycycline.

Embodiment 72: The method of embodiment 65 or 66, wherein the inducing agent comprises a nucleic acid encoding an inducer and a carrier, and wherein the inducer is configured to bind to the inducible promoter, thereby inducing the inducible promoter.

Embodiment 73: The method of embodiment 72, wherein contacting the virus-producing cell with an inducing agent comprises allowing the nucleic acid encoding the inducer to enter the virus-producing cell.

Embodiment 74: The method of embodiment 73, wherein inducing the inducible promoter further comprises expressing the inducer within the virus-producing cell.

Embodiment 75: The method of any one of embodiments 72 to 74, wherein the inducer is a product of a viral helper gene.

Embodiment 76: The method of embodiment 75, wherein the viral helper gene is an adenovirus or herpesvirus helper gene.

Embodiment 77: The method of embodiment 75 or 76, wherein the viral helper gene comprises E1A, E1B55K, E2A, E4orf6, VA RNA, or a combination thereof.

Embodiment 78: The method of embodiment 75 or 76, wherein the viral helper gene comprises UL5, UL8, UL52, ICP8, or a combination thereof.

Embodiment 79: The method of any one of embodiments 72 to 74, wherein the inducible promoter comprises a tetracycline response element (TRE), and the inducer is a tetracycline-controlled transactivator (tTA) or a single-chain tetracycline-controlled transactivator (sctTA).

Embodiment 80: The method of embodiment 79, wherein the nucleic acid comprises SEQ ID NO: 16 or SEQ ID NO: 18.

Embodiment 81: The method of embodiment 79 or 80, wherein the tTA comprises an amino acid sequence of SEQ ID NO: 17.

Embodiment 82: The method of embodiment 79 or 80, wherein the sctTA comprises an amino acid sequence of SEQ ID NO: 19.

Embodiment 83: The method of any one of embodiments 72 to 82, wherein the carrier is a plasmid, a viral particle, or a bead.

Embodiment 84: The method of embodiment 83, wherein the carrier is a plasmid or a bead, and contacting the virus-producing cell with the inducing agent comprises disposing the carrier into the chamber.

Embodiment 85: The method of embodiment 84, wherein disposing the carrier into the chamber is performed by using DEP force, gravity, centrifugation, or a combination thereof.

Embodiment 86: The method of any one of embodiments 26 to 85, wherein the chamber is a first chamber of the microfluidic device, and the microfluidic device comprises a second chamber.

Embodiment 87: The method of embodiment 86, wherein the first chamber comprises a first plurality of virus-producing cells, and the method further comprises preserving a subset of the first plurality of virus-producing cells according to the method of any one of claims 1 to 28, wherein each biological micro-object is a virus-producing cell the plurality of biological micro-objects is a plurality of virus producing cells.

Embodiment 88: The method of embodiment 87 further comprising exporting a third subset of biological micro-objects from the preserving chamber and/or a fourth subset of biological micro-object from the assay chamber.

Embodiment 89: The method of embodiment 88, wherein exporting the third subset of biological micro-objects from the preserving chamber further comprises removing the first in situ-generated cap.

Embodiment 90: The method of embodiment 88 or 89, exporting the third subset of the plurality of biological micro-objects from the preserving chamber further comprises in-situ gelling an interior space of the assay chamber.

Embodiment 91: The method of embodiment 90, wherein the in-situ gelling comprises forming a second in situ-generated cap within the assay chamber.

Embodiment 92: The method of embodiment 90 or 91, wherein the in-situ gelling further comprises forming an in situ-generated structure occupying about 10% to 99% of the interior space of the assay chamber.

Embodiment 93: The method of embodiment 87, wherein the microfluidic device further comprises a third chamber and a fourth chamber, wherein the third chamber comprises a second plurality of virus-producing cells, and the method further comprises preserving a subset of the second plurality of virus-producing cells according to the method of any one of claims 1 to 28, wherein each biological micro-object is a virus-producing cell of the second plurality of virus producing cells.

Embodiment 94: The method of embodiment 93, further comprises allowing the second plurality of virus-producing cells to produce a viral particle in the third chamber.

Embodiment 95: The method of embodiment 94, wherein evaluating a productivity of the virus-producing cell comprises: evaluating a first productivity of the first plurality of virus-producing cells and evaluating a second productivity of the second plurality of virus-producing cells; and selecting a cell of interest from the first plurality of virus-producing cells and the second plurality of virus-producing cells based on comparing the first productivity and the second productivity.

Embodiment 96: The method of embodiment 95, wherein comparing the first productivity and the second productivity comprises comparing the first productivity and the second productivity with a threshold productivity.

Embodiment 97: The method of embodiment 95 or 96, further comprising exporting the selected cell of interest.

Embodiment 98: The method of embodiment 97, wherein exporting the selected cell of interest comprises removing the first in situ-generated cap.

Embodiment 99: The method of embodiment 98, wherein exporting the selected virus-producing cell of interest further comprises: identifying a selected chamber wherein the selected virus-producing cell of interest is located; forming a third in situ-generated cap within a chamber of the microfluidic device except for the selected chamber; and dislodging the selected virus-producing cell of interest from the selected chamber into the flow region.

Embodiment 100: The method of any one of embodiments 26 to 99, wherein the virus-producing cell comprises a mRNA barcode.

Embodiment 101: The method of embodiment 100, further comprising disposing a mRNA capture bead configured to capture the mRNA barcode within the chamber.

Embodiment 102: The method of embodiment 101, further comprising performing reverse transcription.

Embodiment 103: The method of embodiment 101 or 102, further comprising lysing the virus-producing cell.

Embodiment 104: The method of embodiment 103, further comprising, after the lysing, perfusing the microfluidic device with a viscosity gradient flush.

Embodiment 105: The method of embodiment 104, wherein the viscosity gradient flush comprises perfusing the microfluidic device with a first post-lysis buffer comprising 10 to 20% glycerol, and perfusing the microfluidic device with a second post-lysis buffer comprising 5 to 10% glycerol.

Embodiment 106: The method of any one of embodiments 101 to 105, further comprising exporting the mRNA capture bead.

1. A method for preserving a subset of biological micro-objects within a microfluidic device, the method comprising:
moving a first subset of a plurality of biological micro-objects disposed in a first chamber of a microfluidic device into a second chamber of the microfluidic device,
wherein the microfluidic device comprises a microfluidic circuit material defining a flow region and a plurality of chambers fluidically connected to the flow region, wherein the plurality of chambers comprises the first chamber and the second chamber;
designating one of the first chamber and the second chamber as a preserving chamber and the other as an assay chamber; and
forming a first in situ-generated cap within the preserving chamber, wherein the first in situ-generated cap comprises a porosity to selectively block passage of the biological micro-objects between the preserving chamber and the flow region.

2. The method of claim 1, wherein before moving the first subset of the plurality of biological micro-objects, the method further comprises disposing a biological micro-object into the first chamber, and expanding the biological micro-object into the plurality of biological micro-objects.

3. The method of claim 1, wherein moving the first subset of the plurality of biological micro-objects into the second chamber comprises:
selecting one or more biological micro-object(s) from the plurality of biological micro-objects in the first chamber;
moving the selected one or more biological micro-object(s) from the first chamber into the second chamber, thereby forming a first subset of the plurality of biological micro-objects in the second chamber.

4. The method of claim 1, wherein moving the first subset of the plurality of biological micro-objects into the second chamber comprises: moving one or more biological micro-object(s) from the first chamber into a transit area within the flow region and from the transit area into the second chamber, wherein the transit area is substantially enclosed by an in situ-generated barrier, thereby preventing the one or more biological micro-object(s) from entering an area of the flow region other than the transit area while allowing the one or more micro-object(s) to access the transit area, the first chamber, and the second chamber.

5. The method of claim 4, further comprising removing the in situ-generated barrier after moving the first subset of the plurality of micro-objects into the second chamber.

6. The method of claim 1, wherein moving the first subset of the plurality of biological micro-objects into the second chamber comprises using dielectrophoresis (DEP) force, gravity, centrifugation, or a combination thereof.

7. The method of claim 1, wherein the plurality of biological micro-objects is a clonal population.

8. The method of claim 7, wherein moving a first subset of the plurality of biological micro-objects into the second chamber further comprises retaining a second subset of the plurality of biological micro-objects in the first chamber, wherein the first subset and the second subset of biological micro-objects belong to the same clonal population.

9. The method of claim 8, further comprising culturing the first subset of the plurality of biological micro-objects in the second chamber and culturing the second subset of the plurality of micro-objects in the first chamber.

10. The method of claim 1, further comprising performing an assay in the assay chamber.

11. The method of claim 10, wherein the assay is performed after forming the first in situ-generated cap, and wherein the first in situ-generated cap within the preserving chamber prevents the assaying from being performed within the preserving chamber.

12. The method of claim 10, wherein performing an assay in the assay chamber comprises: allowing the first subset of the plurality of biological micro-objects and/or the second subset of the plurality of biological micro-objects to produce a biological product of interest.

13. The method of claim 12, wherein performing an assay in the assay chamber further comprises assaying the biological product of interest.

14. The method of claim 10, wherein performing an assay in the assay chamber comprises:
introducing a lysis buffer into the flow region of the microfluidic device;
diffusing the lysis buffer into the assay chamber; and
blocking the lysis buffer from entering the preserving chamber with the first in situ-generated cap.

15. The method of claim 1, wherein the first in situ-generated cap is moveably connected to one or more surface of the preserving chamber, and/or wherein the first in situ-generated cap comprises a non-uniform thickness with respect to an axis of the chamber such that a portion of the in situ-generated cap is less thick than other portions thereof.

16. The method of claim 1, wherein the first in situ-generated cap comprises a solidified polymer network.

17. The method of claim 16, wherein the solidified polymer network comprises a synthetic polymer, a modified synthetic polymer, a biological polymer, or any combination thereof.

18. The method of claim 16, wherein the solidified polymer network is reversible.

19. A method of evaluating a virus-producing cell within a microfluidic device, the method comprising:
culturing the virus-producing cell in a chamber of the microfluidic device, wherein the microfluidic device comprises a microfluidic circuit material defining a flow region and a chamber, wherein the chamber opens to, and is fluidically connected to, the flow region;
allowing the virus-producing cell to produce a viral particle; and
evaluating a productivity of the virus-producing cell in producing the viral particle, wherein a cell genetically identical to the virus-producing cell, is preserved on the microfluidic device, in a region other than the flow region or the chamber, while evaluating the productivity of the virus-producing cell.

20.-50. (canceled)

51. A method of evaluating a virus-producing cell within a microfluidic device, the method comprising:
disposing the virus-producing cell in a first chamber of the microfluidic device and expanding the virus-producing cell into a plurality of virus-producing cells, moving a first subset of the plurality of virus-producing cells disposed in the first chamber of a microfluidic device into a second chamber of the microfluidic device,
wherein the microfluidic device comprises a microfluidic circuit material defining a flow region and a plurality of chambers fluidically connected to the flow region, wherein the plurality of chambers comprise the first chamber and the second chamber;
designating one of the first chamber and the second chamber as a preserving chamber and the other as an assay chamber; and
forming a first in situ-generated cap within the preserving chamber, wherein the first in situ-generated cap comprises a porosity to selectively block passage of the virus-producing cell between the preserving chamber and the flow region,
allowing the subset of the plurality of virus-producing cells in the assay chamber to produce a plurality of viral particles; and
evaluating a productivity of the virus-producing cell in producing the viral particle.
